(19) United States
(12) Patent Application Publication
(10) Pub. No.: US 2012/0015382 A1
(43) Pub. Date: Jan. 19, 2012

(54) DROPLET-BASED SELECTION

(75) Inventors: David A. Weitz, Bolton, MA (US); Andrew Griffiths, Strasbourg (FR); Sarah Koester, Bad Urach (DE); Vamsi K. Mootha, Cambridge, MA (US); Honey Duan, Xh Den Haag (NL); Jeremy Agresti, Sacramento, CA (US); Christoph Merten, Bottrop (DE); John Heyman, Waltham, MA (US); John R. Gilbert, Brookline, MA (US)

(73) Assignees: President and Fellows of Harvard College, Cambridge, MA (US); The General Hospital Corporation d/b/a Massachusetts General Hospital, Boston, MA (US)

(21) Appl. No.: 13/195,468

(22) Filed: Aug. 1, 2011

Related U.S. Application Data
(63) Continuation of application No. 12/172,186, filed on Jul. 11, 2008.
(60) Provisional application No. 60/959,358, filed on Jul. 13, 2007.
(60) Provisional application No. 61/048,304, filed on Apr. 28, 2008.

(51) Int. Cl. G01N 33/53; G01N 21/64

(52) U.S. Cl. 435/7.92; 435/7.1

(57) ABSTRACT

The present invention generally relates to fluidic droplets, and techniques for screening or sorting such fluidic droplets. In some embodiments, the fluidic droplets may contain cells (e.g., hybridoma cells) that can secrete various species, such as antibodies, for example. In one aspect, a plurality of fluidic droplets containing cells is screened to determine proteins, antibodies, polypeptides, peptides, nucleic acids, or the like. For example, cells able to secrete species such as antibodies may be selected according to certain embodiments of the invention. Examples of such cells include, for instance, immortal cells such as hybridomas, or non-immortal cells such as B-cells. For instance, blood cells may be encapsulated within a plurality of fluidic droplets, and the cells able to produce antibodies may be determined. In some cases, expression or secretion levels may be determined using signaling entities, for example, determinable microparticles present within the fluidic droplet. Other aspects of the invention relate to kits involving such fluidic droplets, methods of promoting the making or use of such fluidic droplets, and the like.

### RELATED APPLICATIONS

This application claims the benefit of U.S. Provisional Patent Application Ser. No. 60/959,358, filed Jul. 13, 2007, entitled “Droplet-Based Selection,” by Weitz, et al., and U.S. Provisional Patent Application Ser. No. 61/048,304, filed Apr. 28, 2008, entitled “Microfluidic Storage and Arrangement of Drops,” by Schmitz, et al. Each of these is incorporated herein by reference.

### GOVERNMENT FUNDING

Research leading to various aspects of the present invention were sponsored, at least in part, by the National Science Foundation, Grant Nos. DMR-0213805, DMR-0602684, and DBI-0649865. The U.S. Government has certain rights in the invention.

### FIELD OF INVENTION

The present invention generally relates to fluidic droplets, and techniques for screening or sorting such fluidic droplets. In some embodiments, the fluidic droplets may contain cells that can secrete various species, such as antibodies, for example, hybridoma cells.

### BACKGROUND

The manipulation of fluids to form fluid streams of desired configuration, discontinuous fluid streams, droplets, particles, dispersions, etc., for purposes of fluid delivery, product manufacture, analysis, and the like, is a relatively well-studied art. For example, highly monodisperse gas bubbles, less than 100 microns in diameter, have been produced using a technique referred to as capillary flow focusing. In this technique, gas is forced out of a capillary tube into a bath of liquid, the tube is positioned above a small orifice, and the contraction flow of the external liquid through this orifice focuses the gas into a thin jet which subsequently breaks into roughly equal-sized bubbles via capillary instability. In a related technique, a similar arrangement can be used to produce liquid droplets in air.

### SUMMARY OF THE INVENTION

The present invention generally relates to fluidic droplets, and techniques for screening or sorting such fluidic droplets. The subject matter of the present invention involves, in some cases, interrelated products, alternative solutions to a particular problem, and/or a plurality of different uses of one or more systems and/or articles.

In one aspect, the invention is directed to a screening method. In one set of embodiments, the method comprises an act of determining a characteristic of a species expressed by a hybridoma contained within a fluidic droplet. In some cases, the fluidic droplet may be one of a plurality of fluidic droplets contained within a liquid, where the droplets have an average dimension of less than about 500 micrometers and a distribution of dimensions such that no more than about 5% of the droplets have a dimension greater than about 10% of the average dimension.

In another set of embodiments, the method includes an act of determining a characteristic of a species present within a fluidic droplet using a signaling entity comprising a microparticle and an agent, immobilized relative to the microparticle, able to bind the species. In some cases, the fluidic droplet may be one of a plurality of fluidic droplets contained within a liquid, where the droplets have an average dimension of less than about 500 micrometers and a distribution of dimensions such that no more than about 5% of the droplets have a dimension greater than about 10% of the average dimension.

In another aspect, the invention is a method. According to a first set of embodiments, the method includes acts of providing a plurality of fluidic droplets contained within a liquid, where at least some of the fluidic droplets contain antibody-producing cells, and culturing the antibody-producing cells to secrete antibodies or portions thereof. In another set of embodiments, the method includes acts of providing a plurality of fluidic droplets contained within a liquid, where at least some of the fluidic droplets contain cells able to secrete a species, and culturing the cells to secrete the species. The method, in yet another set of embodiments, includes acts of providing a plurality of fluidic droplets contained within a liquid, where at least some of the fluidic droplets contain non-immortal cells, and determining a characteristic of a species secreted by the non-immortal cells within the fluidic droplets. The method, in still another set of embodiments, includes acts of providing a plurality of fluidic droplets contained within a liquid, where at least some of the fluidic droplets contain non-immortal cells, and determining a characteristic of a species secreted by the non-immortal cells within the fluidic droplets.

In one set of embodiments, the method includes acts of providing a plurality of fluidic droplets contained within a liquid, where some of the fluidic droplets contain cells able to secrete an species and some of the fluidic droplets contain cells not able to secrete the species, and at least partially separating the fluidic droplets containing the cells able to secrete the species from the fluidic droplets containing the cells not able to secrete the species.

The method, according to another set of embodiments, includes acts of providing a fluidic droplet contained within a liquid, the droplet containing an antibody-producing cell and a target, culturing the antibody-producing cell to secrete antibodies able to recognize the target, and determining association of the antibodies to the target. In still another set of embodiments, the method includes acts of providing a fluidic droplet contained within a liquid, the droplet containing an antibody-producing cell, a first target, an a second target, culturing the antibody-producing cell to secrete antibodies able to recognize at least one of the first target and the second target, and determining a difference in binding between the antibodies and the first and second targets.

The method, in one set of embodiments, includes acts of providing a plurality of fluidic droplets contained within a liquid, at least some of the fluidic droplets containing an antibody-producing cell and a target, where the antibody-producing cells contained within the plurality of fluidic droplets are able to secrete a plurality of distinguishable antibodies and the antibody-producing cells do not all produce the same antibodies, culturing the antibody-producing cell to secrete antibodies within the droplets, and determining, for at least some of the fluidic droplets, association of antibodies contained within the droplet and the target. In another set of embodiments, the method includes acts of providing a plurality of fluidic droplets contained within a liquid, at least some of the fluidic droplets containing an antibody-producing cell, a first target, and a second target, where the antibody-producing cells contained within the plurality of fluidic droplets are able to secrete a plurality of distinguishable antibodies and the antibody-producing cells do not all produce the same antibodies, culturing the antibody-producing cell to secrete antibodies able to recognize at least one of the first cell and the second cell, and determining a difference in binding between the antibodies and the first and second targets.

According to another set of embodiments, the method includes acts of removing blood cells from a subject, encapsulating at least some of the blood cells in a plurality of fluidic droplets, and at least partially separating, from the plurality of fluidic droplets, droplets containing antibody-producing cells. In yet another set of embodiments, the method includes acts of encapsulating blood cells and target cells in a plurality of fluidic droplets, at least partially separating, from the plurality of fluidic droplets, droplets containing blood cells able to produce a species able to associate with the target cell.

In one set of embodiments, the method includes acts of removing blood cells from a subject, encapsulating at least some of the blood cells in a plurality of fluidic droplets, at least partially separating, from the plurality of fluidic droplets, droplets containing antibody-producing cells, sequencing DNA from at least one of the antibody-producing cells, and inserting at least a portion of the DNA in a host cell.

In another aspect, the present invention is directed to a method of making one or more of the embodiments described herein. In another aspect, the present invention is directed to a method of using one or more of the embodiments described herein.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF THE SEQUENCES

SEQ ID NO: 1 is CCPGCC, a Lumio tag.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1 illustrates the production of fluidic droplets, in accordance with one embodiment of the invention;

FIG. 2 illustrates a method of sorting fluidic droplets containing cells, according to another embodiment of the invention;

FIG. 3 illustrates a method of fusing fluidic droplets containing cells, according to yet another embodiment of the invention;

FIG. 4 illustrates a method of forming and fusing fluidic droplets, according to one embodiment of the invention;

FIG. 5 illustrates a method of forming and fusing fluidic droplets, according to one embodiment of the invention;

FIGS. 6A-6I include, according to one set of embodiments, (a) a schematic illustration of single-inlet (left) and double-inlet (right) encapsulation devices; (b) a micrograph of a single-inlet encapsulation device; (c) a micrograph of a double-inlet encapsulation device; (d) a schematic illustration of a serpentine incubation channel (top), a close-up of a serpentine incubation channel (bottom left), and a close-up of an incubation channel for time resolved studies (bottom right); (e) a micrograph of a serpentine incubation channel, (f) a micrograph of a serpentine incubation channel, (g) a schematic illustration of a reinjection device, (h) a micrograph of reinjection for further drop handling, and (i) a micrograph of an incubation channel;

FIGS. 7A-7B include, according to one set of embodiments, (a) a micrograph of single cells encapsulated in drops (with cell-bearing drops highlighted by arrows) and (b) the Poisson distribution for 3 different cell densities where open symbols indicate predicted values from Poisson statistics and solid symbols indicate experimental results;

FIGS. 8A-8C include, according to one set of embodiments, plots of cell survival during incubation in drops. (a) Comparison for survival on chip (6 h, 33 pL drops, n=1167 cells) compared to survival in a culture dish (6 h, n=3681). (b) Survival in a syringe for different drop sizes (3 h, 33 pL: n=319, 21 pL: n=301, 12 pL: n=426). In larger drops survival is increased. On chip survival rates similar to bulk incubation were obtained. (c) Time dependence of cell survival in small drops (12 μL volume, in syringe, 0 h: n=84, 1 h: n=63, 2 h: n=161, 3 h: n=426);

FIGS. 9A-9D include, according to one set of embodiments: (a) A micrograph showing drops containing cells that were encapsulated, incubated for 6 h on chip, recovered from the emulsion and plated. Image was taken after 2 days. (b) A micrograph showing the Control, where cells were grown directly on culture dish. (c) A plot of antibody production in drops. Gray: after three days on culture dish, light green: after first wash, dark green after second wash, orange: encapsulated cells with no incubation time, red: encapsulated cells with 6 h incubation time, blue: cells incubated for 6 h on a culture dish, error bars correspond to the uncertainty in the linear fit to the initial enzyme reaction rate in the kinetic ELISA; and (d) Initial rates of the ELISA for different dilutions of culture supernatant. Color code as in (c). Additional controls (purple, pink): empty emulsion drops, 0 and 6 h incubation time;

FIGS. 10A-10C include a) a schematic illustration of a microfluidic device with a rectangle indicating the section shown in FIG. 10b; (b) a micrograph of drops with encapsulated cells (white scale bar=100 mm); (c) a plot of the experimentally determined probability (p, y axis) for the number of cells per drop (k, x axis). The plot is in good agreement with a Poisson distribution (dashed lines) for various cell densities (resulting from on-chip dilution); and (d) the average number of cells per drop (l) plotted against the cell density for the experimental data (Exp.) and the Poisson distribution (Fit). The dashed line is the theoretical number of cells per drop according to the cell density only (homogeneously distributed); according to one set of embodiments;

FIG. 11 includes, according to one set of embodiments, micrographs of drops comprising cells for multiple surfactants, according to one embodiment of the invention. For each surfactant, the chemical structure and the results of the biocompatibility assay (microscopical bright-field images) are shown. For the assay, HEK293T cells were incubated for 48 hr on a layer of perfluorinated FC40 oil in the presence or absence (control) of the indicated surfactant (0.5% w/w);

FIGS. 12A-12E include, according to one set of embodiments, (a and b) plots of the percentage of viable (a) Jurkat and (b) HEK293T cells recovered from emulsions at the indicated time points; (c) a plot of the total number of recovered Jurkat and HEK293T cells (live and dead) relative to the number of initially encapsulated cells; (d) a plot of the percentage of viable Jurkat cells encapsulated at different densities after 3 d; and (e) a micrograph of HEK293T cells recovered after 48 hr of encapsulation;

FIGS. 13A-13F include, according to one set of embodiments, (a and b) plots of the percentage of viable (a) Jurkat and (b) HEK293T cells recovered from plugs at the indicated time points; (c) a plot of the total number of recovered Jurkat and HEK293T cells (live and dead) relative to the number of initially encapsulated cells; (d) a plot of the percentage of viable Jurkat cells encapsulated at different densities after 3 d; (e) a micrograph of HEK293T cells recovered after 48 hr of encapsulation; and (f) a plot of the mean size of plugs harboring HEK293T cells plotted against the incubation time.

FIG. 14 includes micrographs of the growth of the Nematode C. elegans within droplets, according to one embodiment of the invention;

FIGS. 15A-15F include, according to one set of embodiments, (a) a bright-field image of the inlet during reinjection of an emulsion (drops containing HEK293T cells) after 2 days of incubation; (b) bright-field images of individual drops during encapsulation and after reinjection (off-chip incubation for 2 and 14 d); (c) a fluorescence-microscopic image of drops hosting lacZ-expressing HEK293T cells (converting the fluorogenic substrate FDG) after 16 hr of incubation; (d) a schematic illustration of the optical setup for fluorescence measurements; (e) a plot of the influence of the fluorescence intensity (y axis) on the peak width (w). The peak width is defined as the time (t, x axis) for which a fluorescent signal above a certain threshold (dotted, horizontal line) can be measured (due to a drop passing the laser beam). Different fluorescence intensities of the drops (continuous and dashed peaks) result in different apparent peak widths (w1 and w2); and (f) images and plots of fluorescence signals of drops after reinjection. Upper panels: fluorescence intensity (x axis) plotted against the peak width (y axis) for pure (left) and 1:9 diluted (right) transduced cells. The relative frequency of all events is color coded according to the bar on the right (numbers corresponding to the exponent of the frequency). White gates correspond to noncoalesced drops: left gate, drops considered as negatives; right gate, drops considered as positives. Lower panel: fluorescence intensity (x axis) plotted against the drop counts (y axis) of all events within the gates. Positive events are depicted in red, and negative events are depicted in black;

FIGS. 16A-16C illustrate fluidic mixing in droplets having two or more fluid regions, according to one embodiment of the invention;

FIGS. 17A-17D illustrate uncharged and charged droplets in channels, according to certain embodiments of the invention; and

FIG. 18 is a schematic illustration of screening for antibody-binding to low molecular-weight antigens using fluorescence polarization, according to certain embodiments of the invention. Fluorescent antigens with their absorption transition vectors (arrows) aligned parallel to the electric vector of linearly polarized light (along the vertical page axis) are selectively excited. For small, rapidly rotating antigens, the initially photoselected orientational distribution becomes randomized prior to emission, resulting in low fluorescence polarization. Conversely, binding of the low molecular weight antigen to a large, slowly rotating antibody molecule results in high fluorescence polarization.

### DETAILED DESCRIPTION

The present invention generally relates to fluidic droplets, and techniques for screening or sorting such fluidic droplets. In some embodiments, the fluidic droplets may contain cells (e.g., hybridoma cells) that can secrete various species such as antibodies, for example. In one aspect, a plurality of fluidic droplets containing cells is screened to determine proteins, antibodies, polypeptides, peptides, nucleic acids, or the like. For example, cells able to secrete species such as antibodies may be identified, selected, and/or isolated according to certain embodiments of the invention. Examples of such cells include, for instance, immortal cells such as hybridomas, or non-immortal cells such as B-cells. For instance, blood cells may be encapsulated within a plurality of fluidic droplets, and the cells able to produce antibodies may be determined. In some cases, expression or secretion levels may be determined using signaling entities, for example, determinable microparticles present within the fluidic droplet. Other aspects of the invention relate to kits involving such fluidic droplets, methods of promoting the making or use of such fluidic droplets, and the like.

The following are each incorporated herein by reference: U.S. patent application Ser. No. 11/246,911, filed Oct. 7, 2005, entitled “Formation and Control of Fluidic Species,” published as U.S. Patent Application Publication No. 2006/0163385 on Jul. 27, 2006; U.S. patent application Ser. No. 11/024,228, filed Dec. 28, 2004, entitled “Method and Apparatus for Fluid Dispersion,” published as U.S. Patent Application Publication No. 2005/0172476 on Aug. 11, 2005; U.S. patent application Ser. No. 11/360,845, filed Feb. 23, 2006, entitled “Electronic Control of Fluidic Species,” published as U.S. Patent Application Publication No. 2007/000342 on Jan. 4, 2007; International Patent Application No. PCT/US2006/007772, filed Mar. 3, 2006, entitled “Method and Apparatus for Forming Multiple Emulsions,” published as WO 2006/096571 on Sep. 14, 2006; U.S. patent application Ser. No. 11/368,263, filed Mar. 3, 2006, entitled “Systems and Methods of Forming Particles,” published as U.S. Patent Application Publication No. 2007/0054119 on Mar. 8, 2007; U.S. Provisional Patent Application Ser. No. 60/920,574, filed Mar. 28, 2007, entitled “Multiple Emulsions and Techniques for Formation”; and International Patent Application No. PCT/US2006/001938, filed Jan. 20, 2006, entitled “Systems and Methods for Forming Fluidic Droplets Encapsulated in Particles Such as Colloidal Particles,” published as WO 2006/078841 on Jul. 27, 2006. Also incorporated by reference are U.S. Provisional Patent Application Ser. No. 60/959,358, filed Jul. 13, 2007, entitled “Droplet-Based Selection,” by Weitz, et al., U.S. Provisional Patent Application Ser. No. 61/048,304, filed Apr. 28, 2008, entitled “Microfluidic Storage and Arrangement of Drops,” by Schmitz, et al.; and International Patent Application No. PCT/US2007/017617, filed Aug. 7, 2007, entitled “Fluorocarbon Emulsion Stabilizing Surfactants,” by Weitz, et al.

One aspect of the invention relates to systems and methods for producing droplets of fluid surrounded by a liquid. These fluids can be selected among essentially any fluids by those of ordinary skill in the art by considering the relationship between the fluids. The fluidic droplets may also contain other species in some cases, for example, certain molecular species (e.g., monomers, polymers, metals, etc.), cells, signaling entities, particles, other fluids, or the like. In some cases, the fluid and the liquid may be selected to be immiscible within the time frame of the formation of the fluidic droplets. The fluid and the liquid may be essentially immiscible, i.e., immiscible on a time scale of interest (e.g., the time it takes a fluidic droplet to be transported through a particular system or device). In certain cases, the droplets may each be substantially the same shape and/or size.

As used herein, the term “fluid” generally refers to a substance that tends to flow and to conform to the outline of its container, i.e., a liquid, a gas, a viscoelastic fluid, etc. Typically, fluids are materials that are unable to withstand a static shear stress, and when a shear stress is applied, the fluid experiences a continuing and permanent distortion. The fluid may have any suitable viscosity that permits flow. If two or more fluids are present, each fluid may be independently selected among essentially any fluids (liquids, gases, and the like) by those of ordinary skill in the art, e.g., by considering the relationship between the fluids. The fluids may each be, for example, miscible, slightly miscible, or immiscible. Where the portions remain liquid for a significant period of time, then the fluids may be chosen to be at least substantially immiscible. Those of ordinary skill in the art can select suitable miscible or immiscible fluids, using contact angle measurements or the like, to carry out the techniques of the invention. As used herein, two fluids are immiscible, or not miscible, with each other when one is not soluble in the other to a level of at least 10% by weight at the temperature and under the conditions at which the emulsion is used. For instance, the fluid and the liquid may be selected to be immiscible within the time frame of the formation of the fluidic droplets.

A “fluidic droplet” or a “droplet,” as used herein, is an isolated portion of a first fluid that is completely surrounded by a second fluid. It is to be noted that a fluidic droplet is not necessarily spherical, but may assume other shapes as well, for example, depending on the external environment, the dimensions of the channel or other container that the fluidic droplet is contained within, etc. Examples of a fluidic droplet contained within a liquid include, but are not limited to, a hydrophilic liquid suspended in a hydrophobic liquid, a hydrophobic liquid suspended in a hydrophilic liquid, a gas bubble suspended in a liquid, etc. Typically, a hydrophobic liquid and a hydrophilic liquid are essentially immiscible with respect to each other, where the hydrophilic liquid has a relatively greater affinity to water than does the hydrophobic liquid. Examples of hydrophilic liquids include, but are not limited to, water and other aqueous solutions comprising water, such as cell or biological media, salt solutions, etc., as well as other hydrophilic liquids such as ethanol. Examples of hydrophobic liquids include, but are not limited to, oils such as hydrocarbons, silicone oils, mineral oils, fluorocarbon oils, organic solvents, etc.

In some embodiments, the invention generally relates to an emulsion. The emulsion may include droplets, such as those described above, and/or colloid particles, for example, nanoparticles such as those described below. As used herein, an “emulsion” is given its ordinary meaning as used in the art, e.g., a liquid dispersion. In some cases, the emulsion may be a “microemulsion” or a “nanoemulsion,” i.e., an emulsion having a dispersant on the order of micrometers or nanometers, respectively. As one example, such an emulsion may be created by allowing fluidic droplets of the appropriate size or sizes (e.g., created as described herein) to enter into a solution that is immiscible with the fluidic droplets.

In certain cases, a fluidic stream and/or the fluidic droplets may be produced on the microscale, for example, in a microchannel. Thus, in some, but not all embodiments, at least some of the components of the systems and methods are described herein using terms such as “microfluidic” or “microscale.” As used herein, “microfluidic,” “microscopic,” “microscale,” the “micro-” prefix (for example, as in “microchannel”), and the like generally refers to elements or articles having widths or diameters of less than about 1 mm, and less than about 100 micrometers in some cases. In some cases, the element or article includes a channel through which a fluid can flow. In all embodiments, specified widths can be a smallest width (i.e., a width as specified where, at that location, the article can have a larger width in a different dimension), or a largest width (i.e., where, at that location, the article has a width that is no wider than as specified, but can have a length that is greater). Thus, for example, a fluidic stream may be produced on the microscale, e.g., using a microfluidic channel. For instance, the fluidic stream may have an average cross-sectional dimension of less than about 1 mm, less than about 500 microns, less than about 300 microns, or less than about 100 microns. In some cases, the fluidic stream may have an average diameter of less than about 60 microns, less than about 50 microns, less than about 40 microns, less than about 30 microns, less than about 25 microns, less than about 10 microns, less than about 5 microns, less than about 3 microns, or less than about 1 micron.

A “channel,” as used herein, means a feature on or in an article (e.g., a substrate) that at least partially directs the flow of a fluid. In some cases, the channel may be formed, at least in part, by a single component, e.g., an etched substrate or molded unit. The channel can have any cross-sectional shape, for example, circular, oval, triangular, irregular, square or rectangular (having any aspect ratio), or the like, and can be covered or uncovered (i.e., open to the external environment surrounding the channel). In embodiments where the channel is completely covered, at least one portion of the channel can have a cross-section that is completely enclosed, and/or the entire channel may be completely enclosed along its entire length with the exception of its inlet and outlet.

A channel may have an aspect ratio (length to average cross-sectional dimension) of at least 2:1, more typically at least 3:1, 5:1, 10:1, 30:1, 100:1, 300:1, 1000:1, etc. As used herein, a “cross-sectional dimension,” in reference to a fluidic or microfluidic channel, is measured in a direction generally perpendicular to fluid flow within the channel. An open channel generally will include characteristics that facilitate control over fluid transport, e.g., structural characteristics (an elongated indentation) and/or physical or chemical characteristics (hydrophobicity vs. hydrophilicity) and/or other characteristics that can exert a force (e.g., a containing force) on a fluid. The fluid within the channel may partially or completely fill the channel. In some cases the fluid may be held or confined within the channel or a portion of the channel in some fashion, for example, using surface tension (e.g., such that the fluid is held within the channel within a meniscus, such as a concave or convex meniscus). In an article or substrate, some (or all) of the channels may be of a particular size or less, for example, having a largest dimension perpendicular to fluid flow of less than about 5 mm, less than about 2 mm, less than about 1 mm, less than about 500 microns, less than about 200 microns, less than about 100 microns, less than about 60 microns, less than about 50 microns, less than about 40 microns, less than about 30 microns, less than about 25 microns, less than about 10 microns, less than about 3 microns, less than about 1 micron, less than about 300 nm, less than about 100 nm, less than about 30 nm, or less than about 10 nm or less in some cases. In one embodiment, the channel is a capillary. Of course, in some cases, larger channels, tubes, etc. can be used to store fluids in bulk and/or deliver a fluid to the channel.

In certain embodiments of the invention, the fluidic droplets may contain additional entities, for example, other chemical, biochemical, or biological entities (e.g., dissolved or suspended in the fluid), cells, particles, gases, molecules, or the like. In certain instances, the invention provides for the production of droplets consisting essentially of a substantially uniform number of entities of a species therein (e.g., molecules, cells, particles, etc.). For example, about 90%, about 93%, about 95%, about 97%, about 98%, or about 99%, or more of a plurality or series of droplets may each contain the same number of entities of a particular species. For instance, a substantial number of fluidic droplets produced, e.g., as described above, may each contain 1 entity, 2 entities, 3 entities, 4 entities, 5 entities, 7 entities, 10 entities, 15 entities, 20 entities, 25 entities, 30 entities, 40 entities, 50 entities, 60 entities, 70 entities, 80 entities, 90 entities, 100 entities, etc., where the entities are molecules or macromolecules, cells, particles, etc. Thus, for example, cells (or other entities) may be encapsulated in the plurality of fluidic droplets at an average ratio of no more than about 1 cell/fluidic droplet, 2 cell/fluidic droplet, etc.

In some embodiments, as mentioned, some or all of the fluidic droplets may contain one or more cells (although in other embodiments, the fluidic droplets may be free of cells). The term “cell,” as used herein, is given its ordinary meaning as used in biology. The cell may be an isolated cell, a cell aggregate, or a cell found in a cell culture, in a tissue construct containing cells, or the like. Examples of cells include, but are not limited to, a bacterium (e.g., Escherichia coli), archaeum, or other single-cell organism, a yeast cell (e.g., Saccharomyces cerevisiae), a eukaryotic cell, a plant cell, or an animal cell. If the cell is an animal cell, the cell may be, for example, an invertebrate cell (e.g., a cell from a fruit fly), a fish cell (e.g., a zebrafish cell), an amphibian cell (e.g., a frog cell), a reptile cell, a bird cell, a human cell, or a cell from a non-human mammal, such as a monkey, ape, cow, sheep, goat, buffalo, antelope, oxen, horse, donkey, mule, deer, elk, caribou, water buffalo, a Camelidae (e.g., camels, llamas, alpaca, etc.), rabbit, pig, mouse, rat, guinea pig, hamster, dog, or cat. If the cell is from a multicellular organism, the cell may be from any part of the organism. For instance, if the cell is from an animal, the cell may be, for example, a cardiac cell, a fibroblast, a keratinocyte, a heptaocyte, a chondracyte, a neural cell, an osteocyte, an osteoblast, a muscle cell, a blood cell, an endothelial cell, an immune cell (e.g., a T-cell, a B-cell, a macrophage, a neutrophil, a basophil, a mast cell, an eosinophil), etc. In some embodiments, the cell may be a hematopoietic cell or a cell arising from the blood. In some cases, the cell may be a genetically engineered cell; in other cases, the cell is not genetically engineered. In one set of embodiments, the cell is a hybridoma. In certain embodiments, a fluidic droplet and/or a particular assay may include a combination of two or more cells described herein.

In some cases, the cell may be an immortal cell, while in other cases, the cell may be a non-immortal cell. In general, an immortal cell is generally one that can replicate indefinitely, under suitable conditions without adverse consequences. For instance, a cell that is not limited by the Hayflick limit (where the cell no longer divides because of DNA damage or shortened telomeres) may be immortal. Examples of immortal cells include cancer cells, hybridomas, HeLa cells, HEK cells (e.g., HEK293T) or Jurkat cells. Most naturally occurring cells (for example, blood cells, B cells, plasma cells, etc.), however, are not immortal.

In one aspect, the cell may be a cell able to secrete a species of interest, for example, an antibody, a protein (e.g., a fluorescent protein, such as GFP), a hormone, or the like. The species of interest may be any species secreted by the cell. In one set of embodiments, the cell is an antibody-producing cell. An antibody-producing cell, as used herein, is a cell that secretes antibodies under normal conditions. Non-limiting examples include B-cells (which are non-immortal) and hybridomas (which are generally immortal).

As used herein, an “antibody” refers to a protein or glycoprotein consisting of one or more polypeptides substantially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. A typical immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains respectively.

Antibodies exist as intact immunoglobulins or as a number of well characterized fragments produced by digestion with various peptidases. Thus, for example, pepsin digests an antibody below (i.e. toward the Fc domain) the disulfide linkages in the hinge region to produce F(ab)′2, a dimer of Fab which itself is a light chain joined to V_{H}-C_{H}1 by a disulfide bond. The F(ab)′2 may be reduced under mild conditions to break the disulfide linkage in the hinge region thereby converting the (Fab′)₂ dimer into an Fab′ monomer. The Fab′ monomer is essentially a Fab with part of the hinge region (see, Paul (1993) Fundamental Immunology, Raven Press, N.Y. for a more detailed description of other antibody fragments). While various antibody fragments are defined in terms of the digestion of an intact antibody, one of skill will appreciate that such fragments may be synthesized de novo either chemically, by utilizing recombinant DNA methodology, or by “phage display” methods (see, e.g., Vaughan et al. (1996) Nature Biotechnology, 14(3): 309-314, and PCT/US96/10287). Preferred antibodies include single chain antibodies, e.g., single chain Fv (scFv) antibodies in which a variable heavy and a variable light chain are joined together (directly or through a peptide linker) to form a continuous polypeptide. As specific non-limiting examples, the antibody may be murine (e.g., Orthoclone OKT3, etc.), chimeric (e.g., Rituximab, Remicade, etc.), humanized (e.g., Avastin, Herceptin, etc.), human (e.g., Humira), etc. In some cases, the species comprises a monoclonal antibody, a domain antibody, an antibody fragment (e.g., scFv, Fv, Fab, etc.), or the like.

Various embodiments herein are described with reference to antibodies. However, it should be understood that in some cases, such descriptions also include, as other embodiments, fragments or portions of antibodies. For example, a cell may be contained within a droplet that is able to express a portion of an antibody, for example, a light chain or a heavy chain of an antibody, a fragment of an antibody, etc.

In some cases, the antibody may be one that is selected to have certain desired characteristics, such as the ability to bind to a particular protein (e.g., with a relatively high binding affinity), or even to a particular epitope. For instance, an antibody may bind to a first portion of the protein but not a second portion of the protein, or the antibody may bind to a first protein but not bind to a second protein. In some cases, the second protein may be substantially similar to the first protein, i.e., the antibody may display relatively high specificity to the first protein. Thus, for example, the affinity of the antibody for an antigen or a cell (e.g., relative affinities between different antibodies, absolute affinity, etc.), the off-rate of the antibody from its antigen, the activity of an antibody, and/or the performance of antibodies and/or antibody fragments relative to known therapeutic agents may all be determined in various embodiments.

The cell secreting or producing the antibody (i.e., the antibody-producing cell) may be an immortal or a non-immortal cell. In one embodiment, the antibody-producing cell is a hybridoma cell. For instance, a hybridoma cells are often produced by fusing a non-immortal antibody-producing cell, such as a B-cell, with a tumor cell such as a myeloma tumor cell. The hybridoma cell thus has been genetically engineered or altered. In some cases, however, a non-immortal antibody-producing cell may be desirable. The cell may be one that arises from a subject (e.g., a human), and/or one that has been cultured. The non-immortal antibody-producing cell may be one that is able to produce antibodies under naturally occurring conditions, and often produces “normal” or properly-folded antibodies, even when inside a fluidic droplet as discussed herein.

However, it should be understood that the invention is not limited to only antibody-producing cells. Other cells, e.g., able to secrete a species of interest are contemplated in other embodiments as well. For instance, the cell may secrete a hormone such as insulin (secreted by beta cells), a neurotransmitter such as dopamine or serotonin, a protein or a peptide such as ACTH (adrenocorticotropic hormone) or angiotensin, a messenger such as NO, or the like. As mentioned, the cell may be one that naturally secretes such species, or a cell genetically engineered to secrete the species. For instance, the cell may be a genetically engineered bacteria, such as E. coli.

In some aspects, the fluidic droplets may each be substantially the same shape and/or size (“monodisperse”). For example, the fluidic droplets may have a distribution of dimensions such that no more than about 10% of the fluidic droplets have a dimension greater than about 10% of the average dimension of the fluidic droplets, and in some cases, such that no more than about 8%, about 5%, about 3%, about 1%, about 0.3%, about 0.1%, about 0.03%, or about 0.01% have a dimension greater than about 10% of the average dimension of the fluidic droplets. In some cases, no more than about 5% of the fluidic droplets have a dimension greater than about 5%, about 3%, about 1%, about 0.3%, about 0.1%, about 0.03%, or about 0.01% of the average dimension of the fluidic droplets.

The shape and/or size of the fluidic droplets can be determined, for example, by measuring the average diameter or other characteristic dimension of the droplets. The term “determining,” as used herein, generally refers to the analysis or measurement of a species, for example, quantitatively or qualitatively, and/or the detection of the presence or absence of the species. “Determining” may also refer to the analysis or measurement of an interaction between two or more species, for example, quantitatively or qualitatively, or by detecting the presence or absence of the interaction. Examples of suitable techniques include, but are not limited to, spectroscopy such as infrared, absorption, fluorescence, UV/visible, FTIR (“Fourier Transform Infrared Spectroscopy”), or Raman; gravimetric techniques; ellipsometry; piezoelectric measurements; immunoassays; electrochemical measurements; optical measurements such as optical density measurements; circular dichroism; light scattering measurements such as quasielectric light scattering; polarimetry; refractometry; or turbidity measurements.

The “average diameter” of a plurality or series of droplets is the arithmetic average of the average diameters of each of the droplets. Those of ordinary skill in the art will be able to determine the average diameter (or other characteristic dimension) of a plurality or series of droplets or particles, for example, using laser light scattering, microscopic examination, or other known techniques. The diameter of a droplet, in a non-spherical droplet, is the diameter of a perfect sphere having the same volume as the droplet. The average diameter of a droplet may be, for example, less than about 1 mm, less than about 500 micrometers, less than about 200 micrometers, less than about 100 micrometers, less than about 75 micrometers, less than about 50 micrometers, less than about 40 micrometers, less than about 25 micrometers, less than about 10 micrometers, less than about 5 micrometers, less than about 1 micrometer, less than about 0.3 micrometers, less than about 0.1 micrometers, less than about 0.03 micrometers, or less than about 0.01 micrometers in some cases. The average diameter of the droplet(s) may also be at least about 1 micrometer, at least about 2 micrometers, at least about 3 micrometers, at least about 5 micrometers, at least about 10 micrometers, at least about 15 micrometers, or at least about 20 micrometers in certain cases. The volume may be determined, for example, by impedance measurement, optical techniques (for example a fluorophore of known concentration could be added to the drop-forming media and total amount of that fluorphore could be measured in each drop as an index of volume), microscopy, or the like.

As mentioned, the fluid may be present within the liquid as one or more droplets. In some cases, the droplets may be formed in a device (e.g., a microfluidic device), which allows for the formation of fluidic droplets having a controlled size and/or size distribution. The device may be free of moving parts in some cases. That is, at the location or locations at which fluidic droplets of desired shape and/or size are formed, the device is free of components that move relative to the device as a whole to affect fluidic droplet formation. For example, where fluidic droplets of controlled shape and/or size are formed, the droplets are formed without parts that move relative to other parts of the device that define a channel within which the fluidic droplets flow. This can be referred to as “passive control” or “passive breakup.”

In one example of a passive system, fluid may be urged through a dimensionally-restricted section of a channel of a fluidic device, which can cause the fluid to break up into a series of droplets within the channel. The dimensionally-restricted section can take any of a variety of forms. For example, it can be an annular orifice, elongate, ovoid, square, or the like. Preferably, it is shaped in any way that causes the surrounding liquid to surround and constrict the cross-sectional shape of the fluid being surrounded. The dimensionally-restricted section is non-valved in certain embodiments. That is, it is an orifice that cannot be switched between an open state and a closed state, and typically is of fixed size. One or more intermediate fluid channels can also be provided in some cases to provide an encapsulating fluid surrounding discontinuous portions of fluid being surrounded. Thus, in one embodiment, two intermediate fluid channels are provided, one on each side of a central fluid channel, each with an outlet near the central fluid channel. Control of the fluid flow rate, and ratio between the flow rates of the various fluids within the device, can be used to control the shape and/or size of the fluidic droplets, and/or the monodispersity of the fluidic droplets. The microfluidic devices of the present invention, coupled with the flow rate and ratio control as taught herein, thus may allow significantly improved control and range.

Some embodiments of the present invention involve formation of fluidic droplets in a liquid where the fluidic droplets have a mean cross-sectional dimension no smaller than the mean cross-sectional dimension of the dimensionally-restricted section. The invention, in such embodiments, may involve control over these mean cross-sectional dimensions by control of the flow rate of the fluid, liquid, or both, and/or control of the ratios of these flow rates. In other embodiments, the fluidic droplets have a mean cross-sectional dimension no smaller than about 90% of the mean cross-sectional dimension of the dimensionally-restricted section, and in still other embodiments, no smaller than about 80%, about 70%, about 60%, about 50%, about 40%, or about 30% of the mean cross-sectional dimension of the dimensionally-restricted section.

In another set of embodiments, droplets of fluid can be created in a channel from a fluid surrounded by a liquid by altering the channel dimensions in a manner that is able to induce the fluid to form individual droplets. The channel may, for example, be a channel that expands relative to the direction of flow, e.g., such that the fluid does not adhere to the channel walls and forms individual droplets instead, or a channel that narrows relative to the direction of flow, e.g., such that the fluid is forced to coalesce into individual droplets. In some embodiments, internal obstructions may also be used to cause droplet formation to occur. For instance, baffles, ridges, posts, or the like may be used to disrupt liquid flow in a manner that causes the fluid to coalesce into fluidic droplets. In some cases, the channel dimensions may be altered with respect to time (for example, mechanically, electromechanically, pneumatically, etc.) in such a manner as to cause the formation of individual fluidic droplets to occur. For example, the channel may be mechanically contracted (“squeezed”) to cause droplet formation, or a fluid stream may be mechanically disrupted to cause droplet formation, for example, through the use of moving baffles, rotating blades, or the like.

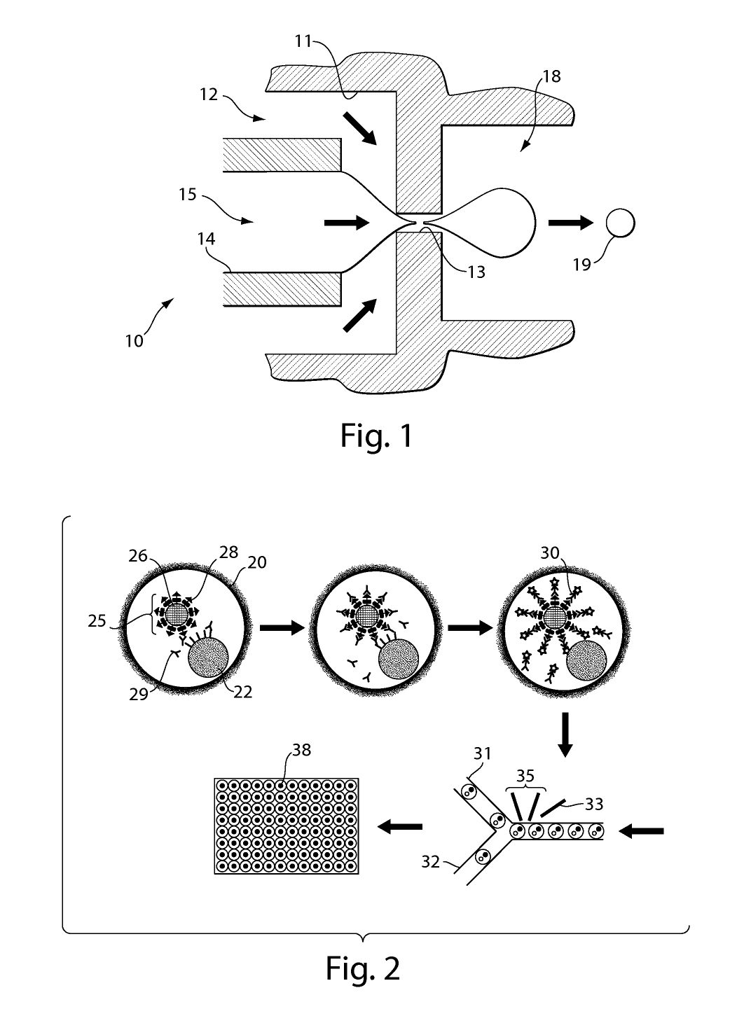

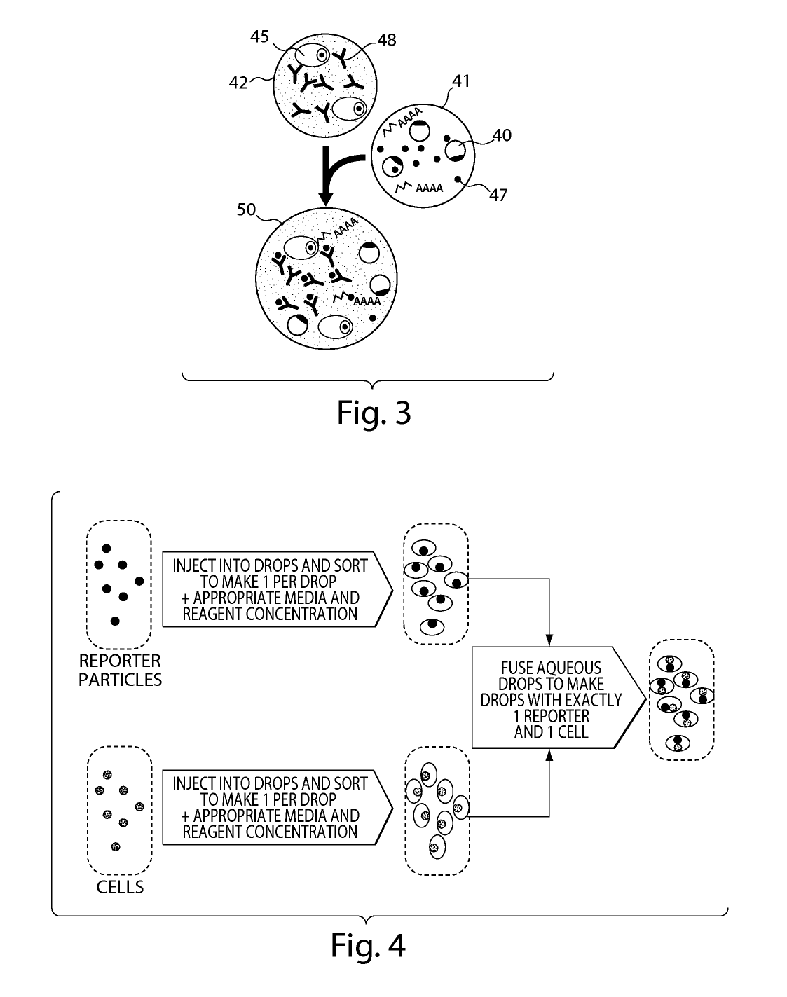

As a non-limiting example of droplet production, a schematic diagram of a device able to produce fluidic droplets is illustrated in FIG. 1. Briefly, a continuous liquid phase 12 is supplied from side channels 11 of the device, and a liquid stream 15 (e.g., containing one or more cells, signaling entitles, etc.) is supplied from a center channel 14. In this geometry, the continuous liquid phase 12 surrounded the inner liquid stream 15; of course, in other embodiments, other arrangements are also possible. The resulting inner liquid stream has an unstable cylindrical morphology, and may break up within dimensional restriction 13 in a generally periodic manner to release fluidic droplets 19 contained within continuous liquid phase 12 into outlet channel 18.

Other techniques of producing droplets of fluid surrounded by a liquid are described in U.S. patent application Ser. No. 11/024,228, filed Dec. 28, 2004, entitled “Method and Apparatus for Fluid Dispersion,” published as U.S. Patent Application Publication No. 2005/0172476 on Aug. 11, 2005; U.S. Patent Application Ser. No. 11/360,845, filed Feb. 23, 2006, entitled “Electronic Control of Fluidic Species,” published as U.S. Patent Application Publication No. 2007/000342 on Jan. 4, 2007; or U.S. patent application Ser. No. 11/368,263, filed Mar. 3, 2006, entitled “Systems and Methods of Forming Particles,” published as U.S. Patent Application Publication No. 2007/0054119 on Mar. 8, 2007, each incorporated herein by reference. For example, in some embodiments, an electric charge may be created on a fluid surrounded by a liquid, which may cause the fluid to separate into individual droplets within the liquid.

In certain embodiments of the invention, the droplets may be produced at relatively high frequencies. For example, the droplets may be formed at frequencies between approximately 100 Hz and 5000 Hz. In some cases, the rate of production may be at least about 200 Hz, at least about 300 Hz, at least about 500 Hz, at least about 750 Hz, at least about 1,000 Hz, at least about 2,000 Hz, at least about 3,000 Hz, at least about 4,000 Hz, or at least about 5,000 Hz. In other embodiments, at least about 10 droplets per second may be produced in some cases, and in other cases, at least about 20 droplets per second, at least about 30 droplets per second, at least about 100 droplets per second, at least about 200 droplets per second, at least about 300 droplets per second, at least about 500 droplets per second, at least about 750 droplets per second, at least about 1000 droplets per second, at least about 1500 droplets per second, at least about 2000 droplets per second, at least about 3000 droplets per second, at least about 5000 droplets per second, at least about 7500 droplets per second, at least about 10,000 droplets per second, at least about 15,000 droplets per second, at least about 20,000 droplets per second, at least about 30,000 droplets per second, at least about 50,000 droplets per second, at least about 75,000 droplets per second, at least about 100,000 droplets per second, at least about 150,000 droplets per second, at least about 200,000 droplets per second, at least about 300,000 droplets per second, at least about 500,000 droplets per second, at least about 750,000 droplets per second, at least about 1,000,000 droplets per second, at least about 1,500,000 droplets per second, at least about 2,000,000 or more droplets per second, or at least about 3,000,000 or more droplets per second may be produced.

In some aspects, the fluidic droplets may also contain additional entities, for example, other chemical, biochemical, or biological entities (which may be dissolved or suspended in the fluid in some cases), for example, monomers, polymers, metals, magnetizable materials, cells, beads, gases, other fluids, or the like. Examples of entities or species that may be contained within, or otherwise associated with, a fluidic droplet include, but are not limited to, signaling entities such as those described below, pharmaceutical agents, drugs, hormones, nucleic acids such as DNA or RNA, proteins (e.g., antibodies), peptides, fragrance, reactive agents, biocides, fungicides, preservatives, chemicals, cells, and the like, as well as combinations thereof. For example, a droplet may contain an antibody-producing cell and an entity which the antibodies produced by the cell can interact with, such as another cell, an antigen, a protein, or the like. Such entities may be useful, for example, in an assay to determine the antibody within the droplet.

Numerous other cell-based assays are possible, including those that monitor cell response to stimuli. For example, cells can be encapsulated with drugs from a drug compound library and assayed for cell death. Additionally or alternatively, target cells can be genetically modified so that a desired antibody binding to a cell surface protein transmits a signal resulting from cellular production of a signaling entity, e.g., green fluorescent protein. These “read-out” cells can be encapsulated with a library of antibody-secreting cells and cells that produce the desired antibody can be isolated and identified.

Thus, in one aspect, a characteristic of a droplet is determined in some fashion, e.g., to determine a species contained within a fluidic droplet. For instance, a species such as a protein, a polypeptide, a peptide, a nucleic acid, an antibody, an enzyme, a virus, a hormone, or the like is determined within the fluidic droplet, and in some cases, the fluidic droplet is processed in some fashion as a result of that determination (e.g., screened and/or sorted, as discussed below).

In one set of embodiments, a signaling entity may be used to determine the characteristic. For instance, a signaling entity may be present within the fluidic droplet and/or within the liquid surrounding the fluidic droplet. Examples of characteristics that may be determined by the signaling entity include, but are not limited to, the presence or concentration of a species, the activity of the species (e.g., the binding activity, catalytic activity, regulatory activity, etc.), and the relative activity of one species compared to another species, etc. In some cases, more than one signaling entity may be used, and in some cases, two or more different, distinguishable signaling entities may be used, e.g., signaling entities able to bind the same or different species. In some embodiments, one or more signaling entities may facilitate the determination of an entity's ability to generate a particular species inside the fluidic droplet (e.g., determination of a cell's ability to produce a particular antibody). In yet other embodiments, one or more signaling entities may facilitate the determination of an entity's response to a particular species (e.g., the response of a cell to a toxin).

As used herein, a “signaling entity” means an entity that is capable of indicating its existence in a particular sample or at a particular location. Signaling entities of the invention can be those that are identifiable by the unaided human eye, those that may be invisible in isolation but may be detectable by the unaided human eye if in sufficient quantity (e.g., microparticles), entities that absorb or emit electromagnetic radiation at a level or within a wavelength range such that they can be readily detected visibly (unaided or with a microscope including an electron microscope or the like), or spectroscopically, or the like. Examples include dyes, pigments, fluorescent moieties (including, by definition, phosphorescent moieties), up-regulating phosphors, chemiluminescent entities, electrochemiluminescent entities, or enzymatic signaling moieties including horseradish peroxidase and alkaline phosphatase.

In one set of embodiments, a signaling entity may comprise a microparticle and an agent immobilized relative to the microparticle that is able to bind, specifically or non-specifically, to a species to be determined, for example, as a protein, a polypeptide, a peptide, a nucleic acid, an antibody, an enzyme, a hormone, or the like. The agent may be immobilized to the microparticle covalently or non-covalently. The agent may be immobilized directly to the microparticle or via a linker. The microparticles typically will have an average diameter (defined as above) of less than about 1 mm, and can be spherical or non-spherical.

In one set of embodiments, the agent is a binding partner of the species to be determined. A “binding partner,” as used herein, refers to any molecule that can undergo binding with a particular molecule. For example, Protein A is a binding partner of the biological molecule IgG, and vice versa. Other non-limiting examples include nucleic acid-nucleic acid binding, nucleic acid-protein binding, protein-protein binding, enzyme-substrate binding, receptor-ligand binding, receptor-hormone binding, antibody-antigen binding, etc. Binding partners include specific, semi-specific, and non-specific binding partners as known to those of ordinary skill in the art. For example, Protein A is usually regarded as a “non-specific” or semi-specific binder.

The term “specifically binds,” when referring to a binding partner (e.g., protein, nucleic acid, antibody, etc.), refers to a reaction that is determinative of the presence and/or identity of one or other member of the binding pair in a mixture of heterogeneous molecules (e.g., proteins and other biologics). Thus, for example, in the case of a receptor/ligand binding pair the ligand would specifically and/or preferentially select its receptor from a complex mixture of molecules, or vice versa. An enzyme would specifically bind to its substrate, a nucleic acid would specifically bind to its complement, an antibody would specifically bind to its antigen. Other examples include nucleic acids that specifically bind (hybridize) to their complement, antibodies specifically bind to their antigen, binding pairs such as those described above, and the like. The binding may be by one or more of a variety of mechanisms including, but not limited to ionic interactions, and/or covalent interactions, and/or hydrophobic interactions, and/or van der Waals interactions, etc.

In one set of embodiments, a first signaling entity may be allowed to bind the species to be determined, and a second signaling entity allowed to bind the first entity. One or both of the first or second signaling entities may be determinable, e.g., fluorescent. Higher-order determinations are also contemplated. For instance, a first signaling entity may be allowed to bind the species to be determined (or another species that is indicative of the species to be determined), and a second signaling entity allowed to bind the first entity, a third signaling entity may be allowed to bind the second entity, etc., and some or all of these entities, may be determinable, e.g., fluorescent.

A non-limiting example of the use of a signaling entity is shown with reference to FIG. 2. In this figure, a fluidic droplet 20 contains a signaling entity 25 and a cell 22. Signaling entity 25 comprises a microparticle 26 and a plurality of agents 28, which may be, for example, a protein, a polypeptide, a peptide, a nucleic acid, an antibody, an enzyme, etc. In some cases, more than one type of agent may be used. Cell 22 may produce a species 29 which is a binding partner to some or all of agents 28. The signaling entities can then be used to determine production of species 29 by cell 22. For instance, if species 29 is expressed on the cell surface, the signaling entities will become associated with the cell, e.g., localized within portions of fluidic droplet 20. If species 29 is released from inside the cell (including by secretion or by lysis of the cell), species 29 may become associated with the signaling entities. As another example, as is shown in FIG. 2, a second signaling entity 30 may be used that is able to bind to species 29. If species 29 is present, second signaling entity 30 may become associated with signaling entity 25 as it binds to species 29; conversely, if species 29 is not present, there may be little or no association of signaling entity 25 and second signaling entity 30. Second signaling entity 30 may be present when droplet 20 is first formed; or, as shown in FIG. 2, second signaling entity 30 can be introduced into droplet 20 by the coalescence of droplet 20 with another fluidic droplet containing signaling entity 30. Non-limiting examples of droplet coalescence are discussed in U.S. patent application Ser. No. 11/246,911, filed Oct. 7, 2005, entitled “Formation and Control of Fluidic Species,” published as U.S. Patent Application Publication No. 2006/0163385 on Jul. 27, 2006; or U.S. patent application Ser. No. 11/360,845, filed Feb. 23, 2006, entitled “Electronic Control of Fluidic Species,” published as U.S. Patent Application Publication No. 2007/000342 on Jan. 4, 2007, each incorporated herein by reference.

In some cases, as is shown in FIG. 2, the droplets may be analyzed to determine species 29, for example, using a sensor as is discussed below. For instance, if species 29 is present in a droplet, the droplet may be sent to a first location 31 (e.g., for further processing, collection as is shown in FIG. 2, or the like); if species 29 is absent (or is present, but in an undesirable amount, concentration, configuration, etc.), the droplet may be sent to a second location 32 (e.g., for further processing, waste, or the like). As shown in FIG. 2, electrodes 35 are used to control movement of the droplets towards first location 31 or second location 32, e.g., as is discussed in U.S. patent application Ser. No. 11/360,845, filed Feb. 23, 2006, entitled “Electronic Control of Fluidic Species,” published as U.S. Patent Application Publication No. 2007/000342 on Jan. 4, 2007, incorporated herein by reference. However, in other embodiments, other systems, e.g., fluidic control, may be used to control the sorting of the droplets. The sensor may include, for example, light (such as a laser) 33 that is directed to the droplets, and the interaction of the light with the droplets may be used to sort or screen the droplets. In some cases, selected droplets can be captured for further analysis, e.g., as is shown in FIG. 2 with array 38. In some embodiments, sorting may be performed as part of a fluorescent-activated cell sorting (FACS) system.

As described herein, one or more signaling entities may be added into the droplets to determine amounts of specific species in the droplet, e.g., molecules produced by a cell (e.g., antibodies) within the droplet, and/or measurement of those species' affinity for binding to a target (e.g., a protein). The signaling entities may also be used, in some cases, to measure those species' relative specificity for binding to one target compared to a second or a third target, for example. Each particular choice of signaling entity may allow, in some embodiments a particular method to implement a screen or selection.

A non-limiting example of a class of signaling entities includes a known quantity of a fluorophore-labeled antigen or “labeled target antigen” (e.g., a FITC labeled phosphopeptide). The labeled target antigen may be contained in a droplet along with a bead coated with a known number of anti-human heavy chain antibodies. In one embodiment, the droplet contains a human B cell that secretes antibodies that bind to both the labeled target antigen and the anti-human heavy chain antibodies on the bead. By measuring the fraction of total fluorophore on the bead, one can measure the affinity of the cell-produced antibody for the target antigen. If a large number of secreted antibodies are bound to the bead, a large fraction of the labeled antigen is on the bead, which shows the secreted antibody has a high affinity for that antigen.

As yet another example, one can add to the droplet a known quantity of an unlabeled related antigen, a “competitor” (e.g., the same labeled target antigen as above but without phosphorylation), which competes with binding to the secreted antibody. The amount of the fluorophore-labeled antigen bound to the bead is reduced if the secreted antibody has significant relative affinity for the competitor.

As still another example, the competitor may be labeled with a third color fluorophore (or second if the tracking agent is not used) so that the ratio of target antigen color to competitor color on the bead is a measure of their relative affinity, and the sum of the two colors is a measure of the amount of secreted antibody on the bead.

The example of the signaling entities above involves, in some cases, binding of an antibody to the bead, for example, through a general anti-heavy chain linker (although other linkers are also possible, as is known to those of ordinary skill in the art). In another embodiment, the target antigen is presented on the surface of the bead, e.g., by covalently linking it to the bead. In this example, the signaling entity may comprise an anti-human heavy chain antibody with a fluorophore label. When one measures that color on the bead, it is a measurement of the amount of cell-secreted antibody that is bound to the target antigen on the bead surface. This example also can be extended to involve the use of a related antigen as a competitor; in this case, the competitor reduces the amount of cell-secreted antibody bound to the bead in direct proportion to the relative affinity of the competitor and the target antigen to the cell-produced antibody.

Many of the methods and articles described herein may involve the use of more than one signaling entity, e.g., two signaling entities that have different colors for two-color detection. For example, in a fluorescence-concentration assay used to select cells which secrete a desired antibody, the signal generated from a large amount of medium-affinity antibody might be similar to the signal generated from a small amount of very high affinity antibody. Two color detection can allow one to simultaneously measure, for example, the amount of secreted antibody and the amount of peptide bound by that antibody. By normalizing the bound peptide signal against the amount of antibody in the droplets, it is possible to accurately rank the antibodies according to binding affinity in some cases.

The present invention provides, in another aspect, a variety of assays and other applications of manipulating droplets containing cells that can secrete various species, such as antibodies, for example, hybridoma cells or non-immortal antibody-producing cells. For instance, droplets may be identified, determined, sorted, split, coalesced with other droplets, reacted, assayed, or the like, and other species may be added to the droplets in some cases. In some cases, such techniques will involve signaling entities or the like, as previously described.

As an example, in one set of embodiments, relatively similar molecules may be differentiated using antibodies or other species. It should be understood that, although cells are described in the context of secreting antibodies, that is only by way of example, and in other embodiments, other cells able to secrete other species (e.g., insulin, neurotransmitters, proteins, hormones, etc.) may be used instead of antibodies and antibody-producing cells.

In one embodiment, an antibody (or other species) may preferentially bind to a first target relative to a second target, even if the targets are substantially similar. For instance, an antibody-producing cell may be co-encapsulated in a fluidic droplet with a first target and a second target, where the antibody-producing cell secretes antibodies having an affinity to the first target and/or the second target. The targets may each be any potentially suitable target for the antibody, for example, a cell, a protein, an enzyme, a virus, or the like. In some cases, a difference in affinity between the antibody and the first target, and the antibody and the second target, may be desirable, and a plurality of fluidic droplets, some of which may contain antibody-producing cells, may be screened to determine those antibody-producing cells having a preferential affinity to the first target relative to the second target.

In one set of embodiments, fluidic droplets that contain at least two different, yet related targets (e.g., steroids with different chemical structures, or phosphorylated versus non-phosphorylated proteins or peptides) may be determined using antibodies or other species. The droplets may contain a species (e.g., an antibody) which can potentially bind to one or more of the targets. A first species may be determined that has a high affinity for one target (e.g., a desired target) but not to a second target (e.g., a competitive binding site that has a similar structure but is inactive). A variety of species (e.g., antibodies) may be tested, e.g., by using a variety of distinguishable cells that secrete the species. For instance, a first droplet may contain a first antibody-producing cell that secretes a first antibody, while a second droplet may contain a second antibody-producing cell that secretes a second antibody distinguishable from the first antibody, e.g., by configuration, sequence, structure, etc. Because each of the species are isolated (e.g., contained in separate droplets), a selectively-binding first species (e.g., that preferentially binds to the first target relative to the second target) can be distinguished from a second species that binds to both targets substantially equally, which may be undesirable. Accordingly, the relative specificity of the species may be determined in some embodiments of the invention.

In one embodiment, droplets containing a species such as an antibody (e.g., produced by an antibody-producing cell) are determined, where the antibody may bind a first target preferentially relative to a second target. For instance, a plurality of droplets may be provided, where at least some of the droplets contain a single B-cell that secretes an antibody (or other species). The secreted antibody may be labeled with a first signaling entity (e.g., a tagged secondary antibody). The droplets may also contain two, three, four, or more target antigens that have a different characteristic, but which may potentially bind to the antibody secreted by the cell. The target antigens may each be labeled with a second signaling entity. In some cases, each of the targets is tagged with a different signaling entity.

To determine whether an antibody in a droplet has a high specificity for a desired target, one can observe the co-localization of signals produced by the signaling entities in each of the droplets. For example, co-localization of the first signaling entity (associated with the secreted antibody) and a second signaling entity associated with a first, desired target indicates that the antibody in this droplet has a high affinity for the desired target. If there are no other co-localized signals in this droplet, this may indicate that the antibody has high selectivity. On the other hand, if the droplet additionally contains co-localization of the first signaling entity with a signaling entity associated with a second target, this may show that the antibody has high affinity but low selectivity. Highly selective species, and cells that secrete such species, can be identified in this manner and then further manipulated if desired. For example, the cells producing such species may be ruptured and the DNA extracted and manipulated to generate replicated antibodies having both high affinity and selectivity for a target, as described herein.

For screens involving cells that secrete antibodies, the cells isolated by this type of screen may produce antibodies that are better functionally-characterized (e.g., have more selective affinity) than, for example, the cells that are isolated after the first steps of a typical hybridoma screen. More complex assays, resulting in more complete antibody characterization, can also be performed. For example, the target protein may be embedded in a lipid bilayer or in a cell membrane and cells can be selected only if the secreted antibodies performed in this context.

In another example, fluidic droplets may contain both a full-length wild-type target protein (e.g., labeled with cy3 dye) and mutant version of the target protein (e.g., a mutant at a key residue in the antibody binding site and labeled with cy5). The screen can identify and select droplets containing cells that secrete an antibody that binds the wild-type protein without binding the mutant protein (in these droplets, the cy3 dye may be concentrated on the protein bead and the cy5 dye may remain diffuse).

In embodiments in which there are at least two different targets inside a fluidic droplet, the targets may be related or non-related. Related targets may include, for example, a first protein or nucleic acid having at least about 70%, at least about 80%, at least about 90%, at least about 95%, at least about 97%, or at least about 99% homology to a second protein or nucleic acid. For instance, a method of the invention may involve providing a fluidic droplet containing two targets, e.g., a first protein and a second protein having at least about 70%, at least about 80%, at least about 90%, at least about 95%, at least about 97%, or at least about 99% homology to the first protein, exposing the droplet to a species such as an antibody able to bind to at least one of the first and second targets, and determining a difference in binding between the species and the first and second targets. This method can be used, for example, to identify cells that produce a particular species with specific binding capabilities (e.g., high affinity and/or high selectivity) in a physiological context. In some cases, the two (or more) targets may have substantially the same compositions or sequences, but the targets may differ in other aspects. For example, the targets may have different secondary structures, different post-translational modifications (for example, phosphorylation, acetylation, etc.), different glycosylation, different epigenetic modifications (for example, methylation), different ionization, or the like.

In another example, related targets may include chemical compounds having similar chemical structures but varying in, for example, less than 10, less than 5, less than 3, or less than 2 functional groups. In some cases, related chemical compounds have a similar chemical structure but vary in molecular weight by less than 30%, less than 20%, less than 15%, less than 10%, less than 5%, or less than 3% (relative to the lighter compound). In some embodiments, related chemical compounds have the same chemical structure but are enantiomers of one another. Other targets may include, for example, a protein, a polypeptide, a peptide, a nucleic acid, an antibody, an enzyme, a virus, a hormone, HIV or other infectious agents (e.g., viruses, bacteria, parasites, prions, etc), and toxic molecules.

It should be understood that the articles and methods described herein can be used to screen for affinity and/or selectivity of a variety of different species of interest within a fluidic droplet. In some cases, the species is introduced into the droplet during formation of the droplet (e.g., the species is a part of the discontinuous phase of the droplet). Sometimes, the species is introduced into the droplet in the absence of a cell. In other cases, the species is secreted by a cell inside the droplet. Non-limiting examples of secreted species include antibodies, hormones, signaling peptides, or the like, as discussed herein. In other embodiments, the species is produced by the cell and is released into the droplet only after rupturing the cell. Non-limiting examples of such species include proteins, polypeptides, peptides, nucleic acids, antibodies, enzymes, hormones, etc., as discussed herein. The cell may be ruptured inside the droplet, in some cases without breaking the droplet, for example. In addition, as described above, a variety of different targets may be contained in the droplet and can be assayed against the species of interest.

Accordingly, a method of screening may comprise, in one embodiment, providing a fluidic droplet contained within a liquid, the droplet containing a first target, a second target, and a cell that can produce a species able to bind with at least one of the first and second targets. The cell can be cultured within the droplet to produce a species of interest, as described herein. Those of ordinary skill in the art will be aware of techniques useful for growing cells in culture, e.g., by exposing the cells to cell culture media, oxygen, carbon dioxide, suitable temperatures, etc. The species may be exposed to the first and second targets in the droplet, e.g., by allowing the cell to secrete the species or by rupturing the cell to release the species. This can result in binding of the species to at least one of the first and/or second targets in the droplet. Additional targets and additional binding events involving the species may also occur in the droplet. Once binding occurs, a difference in binding between the species and the first and second targets can be determined. Additionally, such a method may be conducted for several droplets (e.g., arranged in an array), each droplet containing the same targets but a different cell and/or a different species. By comparing binding events (e.g., using co-localization of signaling entities) between each droplet, a species of interest with desired binding capabilities (e.g., high affinity and/or high selectivity), and, in some cases, the cell that produces the species of interest, can be identified. Furthermore, binding of the species produced by the cell to one target and not the other target may be used to identify a marker specific for a condition (e.g., a marker specific for a disease in an instance where the species binds to a diseased cell but not a healthy cell).

As another example, in one embodiment, a fluidic droplet may contain more than one entity or species in the droplet. For example, a fluidic droplet may contain a cell, a molecule produced (e.g., secreted) by the cell (e.g., an antibody), and a binding molecule (e.g., a cell surface receptor, etc.) able to bind the molecule produced by the cell. Additionally, the fluidic droplet may further contain other entities, for instance, a signaling entity, a second binding molecule that can potentially bind the secreted molecule, etc. In some embodiments, a screening assay may involve the determination of a characteristic of the secreted molecule by observing whether the secreted molecule binds to the first binding molecule and/or second binding molecule (e.g., due to the co-localization of signaling entities associated with each of the species). As described herein, in addition to molecules secreted by a cell, other types of molecules produced by a cell can be screened in this manner.

In one illustrative non-limiting example, a screening assay involves fluidic droplets containing at least three different cells. The cells may include, for example, 1) an antibody-producing cell from an animal immunized with surface proteins purified from cancer cells, 2) a labeled (e.g., cy3-labeled) cancer cell known to have surface markers of interest, and 3) a labeled (e.g., cy5-labeled) healthy cell (lacking the cell surface markers). Antibodies produced by the antibody-producing cell that are secreted within the droplets can be labeled with a third signaling entity (e.g., a fluorescent dye through interaction with an FITC-labeled anti-rabbit antibody). Co-localization of the FITC and cy3 signals brought about by binding between the secreted antibody and the cancer cell (with very low or no co-localization of the FITC and cy5 signals, meaning little or no binding between the antibody and the health cell) would indicate production of a potentially useful marker-specific antibody, while co-localization of FITC with cy3 and cy5 would indicate production of an antibody that binds both healthy and cancerous cells. This example shows that antibodies having different binding affinities/activities, as well as the cells that produce such antibodies, can be identified in physiological conditions using the articles and methods described herein.

As mentioned above, the articles and methods described herein may be used for screening of entities or species, and may include assays such as cell-based assays, non-cell-based assays, antigen capture assays, bioassays (e.g., determination of pharmacological activity of new or chemically undefined substances), competitive protein binding assays, immunoassays, microbiological assays, toxicity assays, and concentration assays, which may be, for example, quantitative or qualitative. Thus, in certain aspects of the invention, one or more characteristics of the fluidic droplets, and/or a characteristic of a portion of the fluidic system containing the fluidic droplet (e.g., the liquid surrounding the fluidic droplet) can be sensed and/or determined in such a manner as to allow the determination of one or more characteristics of the fluidic droplets, for example, using one or more sensors. Characteristics determinable with respect to the droplet and usable in the invention can be identified by those of ordinary skill in the art. Non-limiting examples of such characteristics include fluorescence, spectroscopy (e.g., optical, infrared, ultraviolet, etc.), radioactivity, mass, volume, density, temperature, viscosity, pH, concentration of a substance, such as a biological substance (e.g., a protein, a nucleic acid, etc.), size, shape, color, or the like. In some cases, a fluidic droplet may be screened and/or sorted based on this determination.

As a specific example, a characteristic of a species present within a fluidic droplet (for example, one or more signaling entities, such as those previously described) may be determined in some fashion, and the fluidic droplet screened and/or sorted on the basis of the determination. For instance, the fluidic droplet may contain a cell such as a hybridoma or an antibody-producing cell, and the signaling entity may indicate the presence, concentration, binding activity, catalytic activity, regulatory activity, etc., of a species expressed by the cell, for example, a protein, peptide, nucleic acid, antibody, enzyme, hormone, etc. The fluidic droplet can then be selected or screened on the basis of this determination. As another example, a fluidic droplet may contain a human blood cell, and the fluidic droplet may be selected or screened on the basis of the presence, concentration, etc. of a desired antibody. For example, the fluidic droplet may be directed to a first location (e.g., for further analysis or culture) if the species is present within the fluidic droplet, and to a second location (e.g., to be discarded) if the species is not present within the fluidic droplet, or is present but at an unacceptable level, concentration, configuration, etc. The fluidic droplets may also be further processed, for example, breaking up the fluidic droplet, lysing cells within the droplet, killing cells within the droplets, coalescing the droplets into larger droplets, splitting the droplets into smaller droplets, removing or extracting species from the droplet, adding additional species to the droplet, or the like.

In some systems, such as microfluidic systems, that involve sensing, a sensor may be connected to a processor, which in turn, can cause an operation to be performed on the fluidic droplet, for example, by sorting the droplet, adding or removing electric charge from the droplet, fusing the droplet with another droplet, splitting the droplet, causing mixing to occur within the droplet, etc., for example, as previously described. For instance, in response to a sensor measurement of a fluidic droplet, a processor may cause the fluidic droplet to be split, merged with a second fluidic droplet, etc.

One or more sensors and/or processors may be positioned to be in sensing communication with the fluidic droplet. “Sensing communication,” as used herein, means that the sensor may be positioned anywhere such that the fluidic droplet within the fluidic system (e.g., within a channel), and/or a portion of the fluidic system containing the fluidic droplet may be sensed and/or determined in some fashion. For example, the sensor may be in sensing communication with the fluidic droplet and/or the portion of the fluidic system containing the fluidic droplet fluidly, optically or visually, thermally, pneumatically, electronically, or the like. The sensor can be positioned proximate the fluidic system, for example, embedded within or integrally connected to a wall of a channel, or positioned separately from the fluidic system but with physical, electrical, and/or optical communication with the fluidic system so as to be able to sense and/or determine the fluidic droplet and/or a portion of the fluidic system containing the fluidic droplet (e.g., a channel or a microchannel, a liquid containing the fluidic droplet, etc.). For example, a sensor may be free of any physical connection with a channel containing a droplet, but may be positioned so as to detect electromagnetic radiation arising from the droplet or the fluidic system, such as infrared, ultraviolet, or visible light. The electromagnetic radiation may be produced by the droplet, and/or may arise from other portions of the fluidic system (or externally of the fluidic system) and interact with the fluidic droplet and/or the portion of the fluidic system containing the fluidic droplet in such as a manner as to indicate one or more characteristics of the fluidic droplet, for example, through absorption, reflection, diffraction, refraction, fluorescence, phosphorescence, changes in polarity, phase changes, changes with respect to time, etc. As an example, a laser may be directed towards the fluidic droplet and/or the liquid surrounding the fluidic droplet, and the fluorescence of the fluidic droplet and/or the surrounding liquid may be determined. “Sensing communication,” as used herein may also be direct or indirect. As an example, light from the fluidic droplet may be directed to a sensor, or directed first through a fiber optic system, a waveguide, etc., before being directed to a sensor.

Non-limiting examples of sensors useful in the invention include optical or electromagnetically-based systems. For example, the sensor may be a fluorescence sensor (e.g., stimulated by a laser), a microscopy system (which may include a camera or other recording device), or the like. As another example, the sensor may be an electronic sensor, e.g., a sensor able to determine an electric field or other electrical characteristic. For example, the sensor may detect capacitance, inductance, etc., of a fluidic droplet and/or the portion of the fluidic system containing the fluidic droplet.

As used herein, a “processor” or a “microprocessor” is any component or device able to receive a signal from one or more sensors, store the signal, and/or direct one or more responses (e.g., as described above), for example, by using a mathematical formula or an electronic or computational circuit. The signal may be any suitable signal indicative of the environmental factor determined by the sensor, for example a pneumatic signal, an electronic signal, an optical signal, a mechanical signal, etc.

In still another aspect, the invention provides systems and methods for screening or sorting fluidic droplets in a liquid. Sorting can be accomplished, in some instances, based on the content of a drop (e.g., based on how many particles or cells it contains). In some embodiments, suspensions of aqueous droplets in oil can be prepared that contain a precise number (e.g., one and only one) of particles (e.g., cell, bead, and/or any other particle).

For example, a characteristic of a droplet may be sensed and/or determined in some fashion, then the droplet may be directed towards a particular region of the device, for example, for sorting or screening purposes. For instance, an electric field may be applied or removed from the fluidic droplet to direct the fluidic droplet to a particular region (e.g. a channel). In some cases, high sorting speeds may be achievable using certain systems and methods of the invention. For instance, at least about 10 droplets per second may be determined and/or sorted in some cases, and in other cases, at least about 20 droplets per second, at least about 30 droplets per second, at least about 100 droplets per second, at least about 200 droplets per second, at least about 300 droplets per second, at least about 500 droplets per second, at least about 750 droplets per second, at least about 1000 droplets per second, at least about 1500 droplets per second, at least about 2000 droplets per second, at least about 3000 droplets per second, at least about 5000 droplets per second, at least about 7500 droplets per second, at least about 10,000 droplets per second, at least about 15,000 droplets per second, at least about 20,000 droplets per second, at least about 30,000 droplets per second, at least about 50,000 droplets per second, at least about 75,000 droplets per second, at least about 100,000 droplets per second, at least about 150,000 droplets per second, at least about 200,000 droplets per second, at least about 300,000 droplets per second, at least about 500,000 droplets per second, at least about 750,000 droplets per second, at least about 1,000,000 droplets per second, at least about 1,500,000 droplets per second, at least about 2,000,000 or more droplets per second, or at least about 3,000,000 or more droplets per second may be determined and/or sorted in such a fashion.

In one set of embodiments, a fluidic droplet may be directed by creating an electric charge (e.g., as previously described) on the droplet, and steering the droplet using an applied electric field, which may be an AC field, a DC field, etc. In some cases, the applied electric field may be applied by one or more electrodes proximate the fluidic droplet. In another set of embodiments, a fluidic droplet may be sorted or steered by inducing a dipole in the fluidic droplet (which may be initially charged or uncharged), and sorting or steering the droplet using an applied electric field. The electric field may be an AC field, a DC field, etc.

As an example, an electric field may be selectively applied and removed (or a different electric field may be applied, e.g., a reversed electric field) as needed to direct the fluidic droplet to a particular region. The electric field may be selectively applied and removed as needed, in some embodiments, without substantially altering the flow of the liquid containing the fluidic droplet. For example, a liquid may flow on a substantially steady-state basis (i.e., the average flowrate of the liquid containing the fluidic droplet deviates by less than 20% or less than 15% of the steady-state flow or the expected value of the flow of liquid with respect to time, and in some cases, the average flowrate may deviate less than 10% or less than 5%) or other predetermined basis through a fluidic system of the invention (e.g., through a channel or a microchannel), and fluidic droplets contained within the liquid may be directed to various regions, e.g., using an electric field, without substantially altering the flow of the liquid through the fluidic system.

In another embodiment, the fluidic droplets may be screened or sorted within a fluidic system of the invention by altering the flow of the liquid containing the droplets. For instance, in one set of embodiments, a fluidic droplet may be steered or sorted by directing the liquid surrounding the fluidic droplet into a first channel, a second channel, etc.

In another set of embodiments, pressure within a fluidic system, for example, within different channels or within different portions of a channel, can be controlled to direct the flow of fluidic droplets. For example, a droplet can be directed toward a channel junction including multiple options for further direction of flow (e.g., directed toward a branch, or fork, in a channel defining optional downstream flow channels). Pressure within one or more of the optional downstream flow channels can be controlled to direct the droplet selectively into one of the channels, and changes in pressure can be effected on the order of the time required for successive droplets to reach the junction, such that the downstream flow path of each successive droplet can be independently controlled. In one arrangement, the expansion and/or contraction of liquid reservoirs may be used to steer or sort a fluidic droplet into a channel, e.g., by causing directed movement of the liquid containing the fluidic droplet. The liquid reservoirs may be positioned such that, when activated, the movement of liquid caused by the activated reservoirs causes the liquid to flow in a preferred direction, carrying the fluidic droplet in that preferred direction. For instance, the expansion of a liquid reservoir may cause a flow of liquid towards the reservoir, while the contraction of a liquid reservoir may cause a flow of liquid away from the reservoir. In some cases, the expansion and/or contraction of the liquid reservoir may be combined with other flow-controlling devices and methods, e.g., as described herein. Non-limiting examples of devices able to cause the expansion and/or contraction of a liquid reservoir include pistons and piezoelectric components. In some cases, piezoelectric components may be particularly useful due to their relatively rapid response times, e.g., in response to an electrical signal.

In some embodiments, the fluidic droplets may be sorted into more than two channels, and in certain cases, a fluidic droplet may be sorted and/or split into two or more separate droplets, for example, depending on the particular application. Any of the above-described techniques may be used to split and/or sort droplets. As a non-limiting example, by applying (or removing) a first electric field to a device (or a portion thereof), a fluidic droplet may be directed to a first region or channel; by applying (or removing) a second electric field to the device (or a portion thereof), the droplet may be directed to a second region or channel; by applying a third electric field to the device (or a portion thereof), the droplet may be directed to a third region or channel; etc., where the electric fields may differ in some way, for example, in intensity, direction, frequency, duration, etc. In a series of droplets, each droplet may be independently sorted and/or split; for example, some droplets may be directed to one location or another, while other droplets may be split into multiple droplets directed to two or more locations.

Additional examples of screening or sorting fluidic droplets are disclosed in U.S. patent application Ser. No. 11/360,845, filed Feb. 23, 2006, entitled “Electronic Control of Fluidic Species,” published as U.S. Patent Application Publication No. 2007/000342 on Jan. 4, 2007, incorporated herein by reference.

In still another aspect, one or more fluidic droplets may be fused with other fluidic droplets, for example, to introduce and mix the contents of one droplet with another. One example set of embodiments is illustrated in FIG. 4. In this set of embodiments, a fluidic droplet comprising one or more cells may be fused with a fluidic droplet comprising a signaling entity (e.g., a bead) to introduce a cell to the signaling entity. In some cases, the microfluidic systems described herein may be used to accomplish the fusing step, as described in more detail below. Examples of such systems include those described in, for example, in U.S. Patent Application Ser. No. 11/360,845, filed Feb. 23, 2006, entitled “Electronic Control of Fluidic Species,” published as U.S. Patent Application Publication No. 2007/000342 on Jan. 4, 2007, incorporated herein by reference.

In the embodiments illustrated in FIG. 4, a microfluidic system takes as one input an aqueous suspensions of cells and as another input an aqueous suspension of beads to be used as part of a signaling entity. In addition, controlled fusion of a droplet containing one bead and a droplet containing one cell is performed in the microfluidic system to make a suspension or stream of droplets containing exactly one cell and one bead. In some cases, the system can produce droplets with any number of cells and/or beads. In some embodiments, such a system could prepare controlled mixtures of cell types.

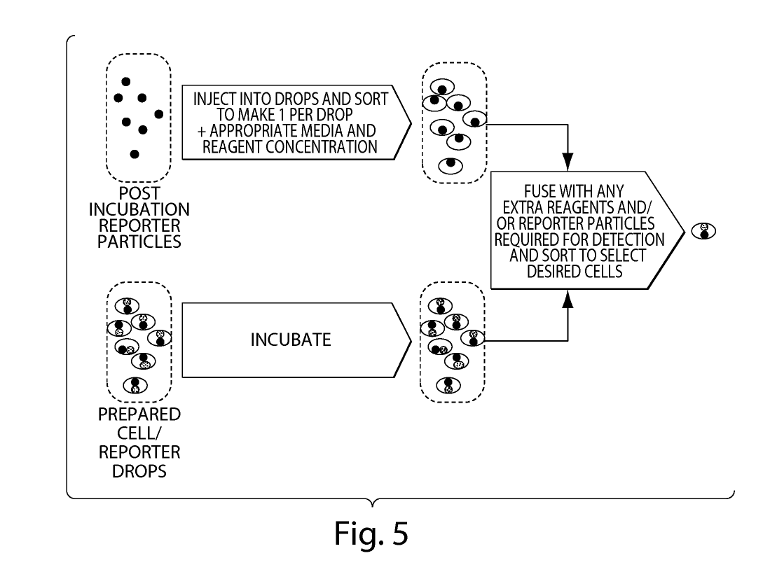

As another example, illustrated in FIG. 5, a droplet comprising a cell and a signaling entity may be fused with another droplet comprising a second signaling entity. In some instances, this step may be performed after a preparation step similar to that illustrated in FIG. 4. In the set of embodiments illustrated in FIG. 5, the prepared cells may be incubated for an appropriate period according to their nature (since, for instance, different cell types may need different incubation times). In some embodiments, controlled fusion may be performed to merge a droplet comprising a cell and a signaling entity with a droplet comprising other reagents, signaling entities, cells, etc. In some cases, analysis of the fused droplet may be used to select and/or sort desired droplets, which can be used, for example, to isolate one or more cells, such as antibody-producing cells.

One of ordinary skill in the art will understand that FIGS. 4 and 5 offer a representative example schematic for a broad class of similar operations, and accordingly should not be considered to be limiting. In some cases, pre-incubation reporters will not be required. In some instances, analysis may be performed without post-incubation, for example.

In one set of embodiments, two or more fluidic droplets, such as those described above, may be fused or coalesced into one droplet. For example, in one set of embodiments, systems and methods are provided that are able to cause two or more droplets (e.g., arising from discontinuous streams of fluid) to fuse or coalesce into one droplet. In some cases, the two or more droplets ordinarily are unable to fuse or coalesce due to, for example, composition, surface tension, droplet size, the presence or absence of surfactants, etc. In certain microfluidic systems, the surface tension of the droplets, relative to the size of the droplets, may also prevent fusion or coalescence of the droplets from occurring in some cases.

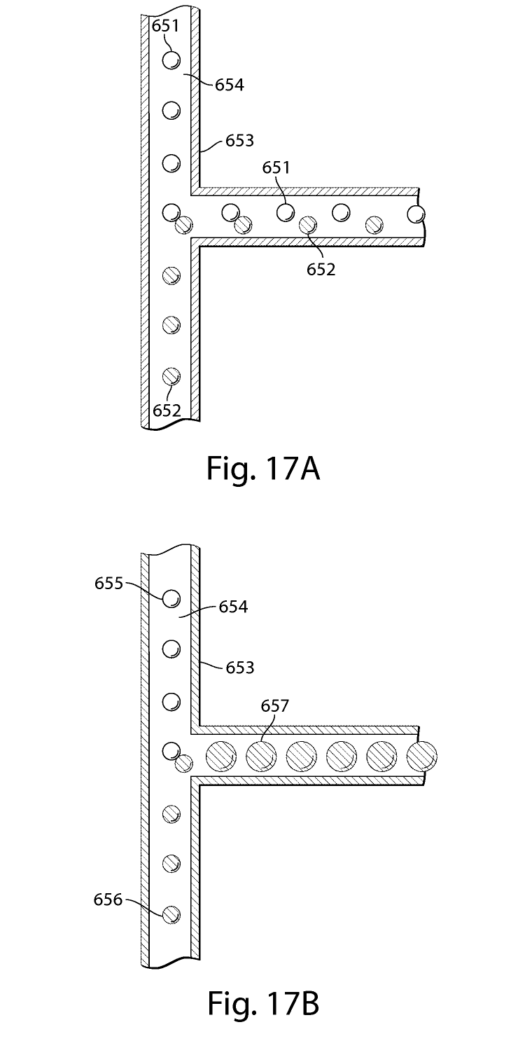

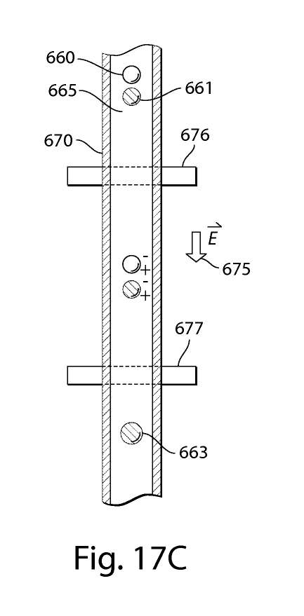

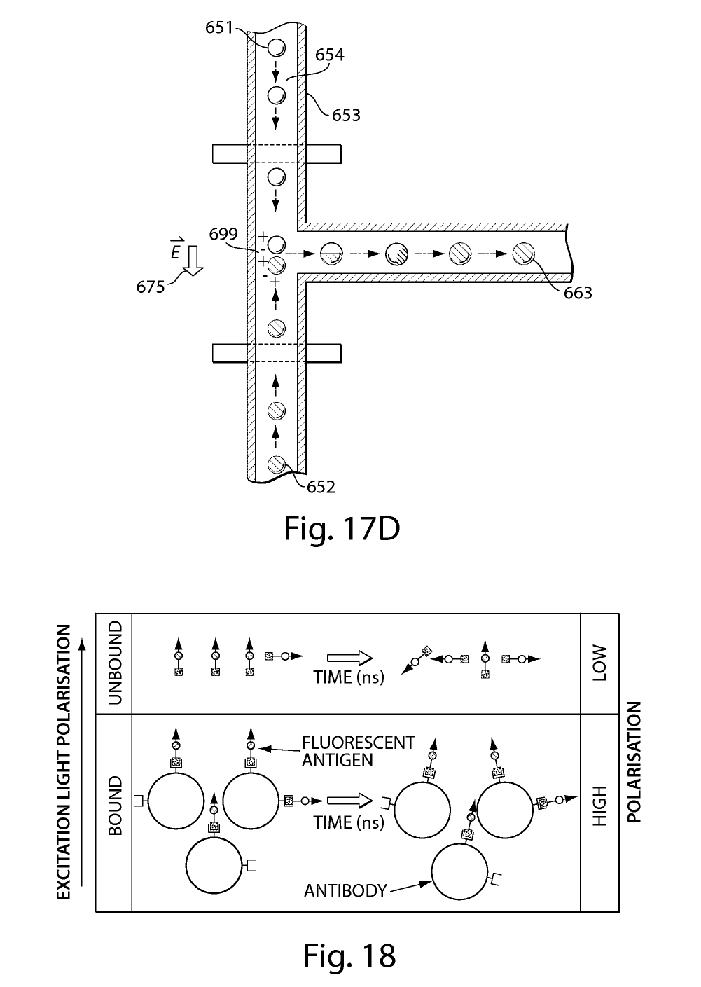

In one embodiment, two fluidic droplets may be given opposite electric charges (i.e., positive and negative charges, not necessarily of the same magnitude), which may increase the electrical interaction of the two droplets such that fusion or coalescence of the droplets can occur due to their opposite electric charges, e.g., using the techniques described herein. For instance, an electric field may be applied to the droplets, the droplets may be passed through a capacitor, a chemical reaction may cause the droplets to become charged, etc. As an example, as is shown schematically in FIG. 17A, uncharged droplets 651 and 652, carried by a liquid 654 contained within a microfluidic channel 653, are brought into contact with each other, but the droplets are not able to fuse or coalesce, for instance, due to their size and/or surface tension. The droplets, in some cases, may not be able to fuse even if a surfactant is applied to lower the surface tension of the droplets. However, if the fluidic droplets are electrically charged with opposite charges (which can be, but are not necessarily of, the same magnitude), the droplets may be able to fuse or coalesce. For instance, in FIG. 17B, positively charged droplets 655 and negatively charged droplets 656 are directed generally towards each other such that the electrical interaction of the oppositely charged droplets causes the droplets to fuse into fused droplets 657.

In another embodiment, the fluidic droplets may not necessarily be given opposite electric charges (and, in some cases, may not be given any electric charge), and are fused through the use of dipoles induced in the fluidic droplets that causes the fluidic droplets to coalesce. In the example illustrated in FIG. 17C, droplets 660 and 661 (which may each independently be electrically charged or neutral), surrounded by liquid 665 in channel 670, move through the channel such that they are the affected by an applied electric field 675. Electric field 675 may be an AC field, a DC field, etc., and may be created, for instance, using electrodes 676 and 677, as shown here. The induced dipoles in each of the fluidic droplets, as shown in FIG. 17C, may cause the fluidic droplets to become electrically attracted towards each other due to their local opposite charges, thus causing droplets 660 and 661 to fuse to produce droplet 663. In FIG. 17D, droplets 660 and 661 approach each other from opposite directions. Droplets 660 and 661 are affected by an applied electric field, and dipoles are induced in each of the fluidic droplets. As shown in FIG. 17D, droplets 651 and 652 meet at point 699 and are fused to create droplet 663.

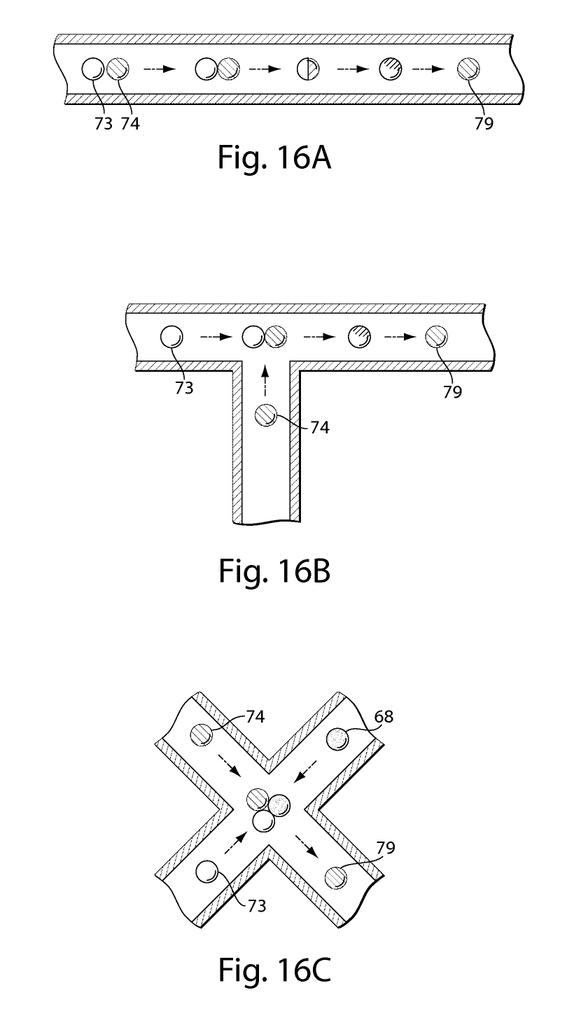

It should be noted that, in various embodiments, the two or more droplets allowed to coalesce are not necessarily required to meet “head-on.” Any angle of contact, so long as at least some fusion of the droplets initially occurs, is sufficient. As an example, in FIG. 16A, droplets 73 and 74 each are traveling in substantially the same direction (e.g., at different velocities), and are able to meet and fuse. As another example, in FIG. 16B, droplets 73 and 74 meet at an angle and fuse. In FIG. 16C, three fluidic droplets 73, 74 and 68 meet and fuse to produce droplet 79.

It should be noted that when two or more droplets “coalesce,” perfect mixing of the fluids from each droplet in the resulting droplet does not instantaneously occur. In some cases, the fluids may not mix, react, or otherwise interact, thus resulting in a fluid droplet where each fluid remains separate or at least partially separate. In other cases, the fluids may each be allowed to mix, react, or otherwise interact with each other, thus resulting in a mixed or a partially mixed fluid droplet. In some cases, the coalesced droplets may be contained within a carrying fluid, for example, an oil in the case of aqueous droplets.

Other examples of fusing or coalescing fluidic droplets are described in International Patent Application Serial No. PCT/US2004/010903, filed Apr. 9, 2004 by Link, et al. and International Patent Application Serial No. PCT/US2004/027912, filed Aug. 27, 2004 by Link, et al., incorporated herein by reference.

A variety of materials and methods, according to certain aspects of the invention, can be used to form the fluidic or microfluidic system. For example, various components of the invention can be formed from solid materials, in which the channels can be formed via micromachining, film deposition processes such as spin coating and chemical vapor deposition, laser fabrication, photolithographic techniques, etching methods including wet chemical or plasma processes, and the like. See, for example, Scientific American, 248:44-55, 1983 (Angell, et al).

In one set of embodiments, at least a portion of the fluidic system is formed of silicon by etching features in a silicon chip. Technologies for precise and efficient fabrication of various fluidic systems and devices of the invention from silicon are known. In another embodiment, various components of the systems and devices of the invention can be formed of a polymer, for example, an elastomeric polymer such as polydimethylsiloxane (“PDMS”), polytetrafluoroethylene (“PTFE” or Teflon®), or the like. For instance, according to one embodiment, system 10 shown in FIG. 1 may be implemented by fabricating the fluidic system separately using PDMS or other soft lithography techniques (details of soft lithography techniques suitable for this embodiment are discussed in the references entitled “Soft Lithography,” by Younan Xia and George M. Whitesides, published in the Annual Review of Material Science, 1998, Vol. 28, pages 153-184, and “Soft Lithography in Biology and Biochemistry,” by George M. Whitesides, Emanuele Ostuni, Shuichi Takayama, Xingyu Jiang and Donald E. Ingber, published in the Annual Review of Biomedical Engineering, 2001, Vol. 3, pages 335-373; each of these references is incorporated herein by reference).

Different components can be fabricated of different materials. For example, a base portion including a bottom wall and side walls can be fabricated from an opaque material such as silicon or PDMS, and a top portion can be fabricated from a transparent or at least partially transparent material, such as glass or a transparent polymer, for observation and/or control of the fluidic process. Components can be coated so as to expose a desired chemical functionality to fluids that contact interior channel walls, where the base supporting material does not have a precise, desired functionality. For example, components can be fabricated as illustrated, with interior channel walls coated with another material. Material used to fabricate various components of the systems and devices of the invention, e.g., materials used to coat interior walls of fluid channels, may desirably be selected from among those materials that will not adversely affect or be affected by fluid flowing through the fluidic system, e.g., material(s) that is chemically inert in the presence of fluids to be used within the device.

In some embodiments, various components of the invention are fabricated from polymeric and/or flexible and/or elastomeric materials, and can be conveniently formed of a hardenable fluid, facilitating fabrication via molding (e.g. replica molding, injection molding, cast molding, etc.). The hardenable fluid can be essentially any fluid that can be induced to solidify, or that spontaneously solidifies, into a solid capable of containing and/or transporting fluids contemplated for use in and with the fluidic network. In one embodiment, the hardenable fluid comprises a polymeric liquid or a liquid polymeric precursor (i.e. a “prepolymer”). Suitable polymeric liquids can include, for example, thermoplastic polymers, thermoset polymers, or mixture of such polymers heated above their melting point. As another example, a suitable polymeric liquid may include a solution of one or more polymers in a suitable solvent, which solution forms a solid polymeric material upon removal of the solvent, for example, by evaporation. Such polymeric materials, which can be solidified from, for example, a melt state or by solvent evaporation, are well known to those of ordinary skill in the art. A variety of polymeric materials, many of which are elastomeric, are suitable, and are also suitable for forming molds or mold masters, for embodiments where one or both of the mold masters is composed of an elastomeric material. A non-limiting list of examples of such polymers includes polymers of the general classes of silicone polymers, epoxy polymers, and acrylate polymers. Epoxy polymers are characterized by the presence of a three-membered cyclic ether group commonly referred to as an epoxy group, 1,2-epoxide, or oxirane. For example, diglycidyl ethers of bisphenol A can be used, in addition to compounds based on aromatic amine, triazine, and cycloaliphatic backbones. Another example includes the well-known Novolac polymers. Non-limiting examples of silicone elastomers suitable for use according to the invention include those formed from precursors including the chlorosilanes such as methylchlorosilanes, ethylchlorosilanes, phenylchlorosilanes, etc.

Silicone polymers are used in certain embodiments, for example, the silicone elastomer polydimethylsiloxane. Non-limiting examples of PDMS polymers include those sold under the trademark Sylgard by Dow Chemical Co., Midland, Mich., and particularly Sylgard 182, Sylgard 184, and Sylgard 186. Silicone polymers including PDMS have several beneficial properties simplifying fabrication of the microfluidic structures of the invention. For instance, such materials are inexpensive, readily available, and can be solidified from a prepolymeric liquid via curing with heat. For example, PDMSs are typically curable by exposure of the prepolymeric liquid to temperatures of about, for example, about 65° C. to about 75° C. for exposure times of, for example, about an hour. Also, silicone polymers, such as PDMS, can be elastomeric and thus may be useful for forming very small features with relatively high aspect ratios, necessary in certain embodiments of the invention. Flexible (e.g., elastomeric) molds or masters can be advantageous in this regard.

One advantage of forming structures such as microfluidic structures of the invention from silicone polymers, such as PDMS, is the ability of such polymers to be oxidized, for example by exposure to an oxygen-containing plasma such as an air plasma, so that the oxidized structures contain, at their surface, chemical groups capable of cross-linking to other oxidized silicone polymer surfaces or to the oxidized surfaces of a variety of other polymeric and non-polymeric materials. Thus, components can be fabricated and then oxidized and essentially irreversibly sealed to other silicone polymer surfaces, or to the surfaces of other substrates reactive with the oxidized silicone polymer surfaces, without the need for separate adhesives or other sealing means. In most cases, sealing can be completed simply by contacting an oxidized silicone surface to another surface without the need to apply auxiliary pressure to form the seal. That is, the pre-oxidized silicone surface acts as a contact adhesive against suitable mating surfaces. Specifically, in addition to being irreversibly sealable to itself, oxidized silicone such as oxidized PDMS can also be sealed irreversibly to a range of oxidized materials other than itself including, for example, glass, silicon, silicon oxide, quartz, silicon nitride, polyethylene, polystyrene, glassy carbon, and epoxy polymers, which have been oxidized in a similar fashion to the PDMS surface (for example, via exposure to an oxygen-containing plasma). Oxidation and sealing methods useful in the context of the present invention, as well as overall molding techniques, are described in the art, for example, in an article entitled “Rapid Prototyping of Microfluidic Systems and Polydimethylsiloxane,” Anal. Chem., 70:474-480, 1998 (Duffy et al.), incorporated herein by reference.

Another advantage to forming microfluidic structures of the invention (or interior, fluid-contacting surfaces) from oxidized silicone polymers is that these surfaces can be much more hydrophilic than the surfaces of typical elastomeric polymers (where a hydrophilic interior surface is desired). Such hydrophilic channel surfaces can thus be more easily filled and wetted with aqueous solutions than can structures comprised of typical, unoxidized elastomeric polymers or other hydrophobic materials.

In one embodiment, a bottom wall is formed of a material different from one or more side walls or a top wall, or other components. For example, the interior surface of a bottom wall can comprise the surface of a silicon wafer or microchip, or other substrate. Other components can, as described above, be sealed to such alternative substrates. Where it is desired to seal a component comprising a silicone polymer (e.g. PDMS) to a substrate (bottom wall) of different material, the substrate may be selected from the group of materials to which oxidized silicone polymer is able to irreversibly seal (e.g., glass, silicon, silicon oxide, quartz, silicon nitride, polyethylene, polystyrene, epoxy polymers, and glassy carbon surfaces which have been oxidized). Alternatively, other sealing techniques can be used, as would be apparent to those of ordinary skill in the art, including, but not limited to, the use of separate adhesives, thermal bonding, solvent bonding, ultrasonic welding, etc.

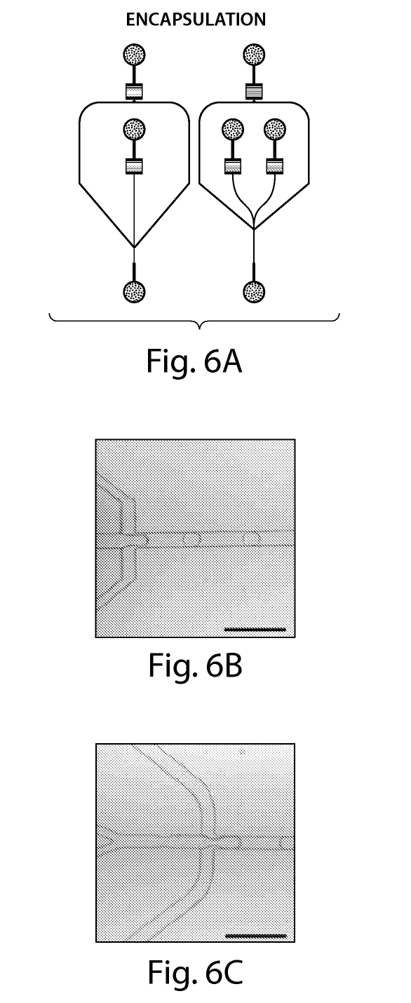

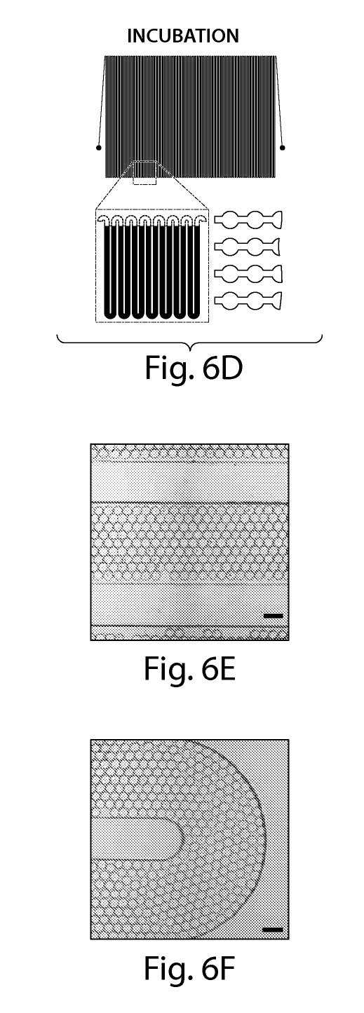

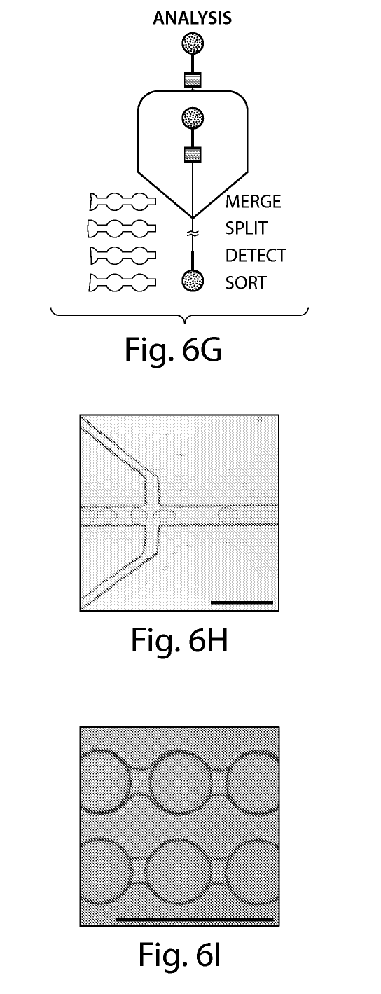

Certain embodiments of the present invention involve the use of systems and methods for the arrangement of droplets in pre-determined locations. In some embodiments, the invention can interface not only with microfluidic/microscale equipment, but with macroscopic equipment to allow for the easy injection of liquids and extraction of sample droplets, etc. In one set of embodiments, a device can be used that comprises one or more “pots” (as shown, for example, in FIG. 6i) into which individual droplets can be transported and stored. In one embodiment, a droplet is urged through a constriction in a storage channel into a pot. Once in the pot, the droplet may remain stably positioned, or it may be urged from the pot through a second constriction and/or through further constrictions into and/or through various pots which can identical or similar to, or different from, the original pot. Systems and methods for the arrangement of droplets are described in U.S. Provisional Patent Application Ser. No. 61/048,304, filed Apr. 28, 2008, entitled “Microfluidic Storage and Arrangement of Drops,” which is incorporated herein by reference.

In yet another aspect, articles and methods are described herein that can be used for direct screening of cells taken from a subject, such as a human. A “subject,” as used herein, means a human or non-human animal. Examples of subjects include, but are not limited to, a mammal such as a dog, a cat, a horse, a donkey, a mule, a deer, an elk, a caribou, a llama, an alpaca, an antelope, a rabbit, a cow, a pig, a sheep, a goat, a rat (e.g., Rattus Norvegicus), a mouse (e.g., Mus musculus), a guinea pig, a hamster, a primate (e.g., a monkey, a chimpanzee, a baboon, an ape, a gorilla, etc.), or the like; a bird such as a chicken, a turkey, a quail, etc.; a reptile (e.g., a snake); an amphibian such as a toad, a frog (e.g., Xenopus laevis), etc.; a fish such as a zebrafish (e.g., Danio rerio); or the like. For example, in one embodiment, cells are taken from a subject, e.g., from the blood of the subject. The blood cells (or other cells) are then screened, for example, as described herein, to determine one or more antibody-producing cells or other cells able to secrete a species.

The screening process can allow identification and selection of the cells that produce these antibodies, and these cells and antibodies may then serve as building blocks for therapeutics, as discussed below. In another example, useful antibody-producing cells from human subjects can be screened. For instance, the subject may be one that was exposed to and/or who can make useful antibodies against an agent of interest such as HIV or other infectious agents (e.g., viruses, bacteria, parasites, prions, etc). Similarly, some humans may produce antibodies against toxic molecules such as drugs of abuse or other toxins, and these antibodies can be isolated using methods and articles described herein. It should be noted that the subject is not necessarily one that appears sick. The subject may be healthy, but produce antibodies of interest (e.g., against an infectious agent, such as HIV). As another example, cancer patients may produce antibodies specific to cancer-cell surface markers. By identifying or determining the antibody-producing cells that produce antibodies against an agent of interest, such antibodies may be produced, as discussed in detail below, and administered to the subject and/or to other subjects, depending on the application.

It should be noted that, in the descriptions herein, cells are screened on the basis of their production of antibodies. However, it should be understood that this is by way of example only, and in other embodiments, other cells able to secrete other species (e.g., insulin, neurotransmitters, proteins, hormones, etc.) may be studied instead of antibodies and antibody-producing cells. Similarly, although the cells are described in the examples below as arising from the blood of a subject or from culture, in other embodiments, the cells may arise from other sources as well, for example, bodily fluids, biopsies, or the like. Further non-limiting examples include tissue biopsies, serum or other blood fractions, urine, ocular fluid, saliva, cerebro-spinal fluid, fluid or other samples from tonsils, lymph nodes, needle biopsies, etc.

In some embodiments, the cells may be used as part of a treatment (e.g., of an autoimmune disease). As an example, cells (e.g., human blood cells) that produce desired antibodies may be identified and/or sorted. The cells may then be cultured, in some cases, to produce antibodies which may, for example, be harvested and introduced into a subject. In some cases, the antibody-producing cells may be cultured and given to the subject directly.

A method of screening according to one embodiment may involve, for example, providing a plurality of B cells from a human (e.g., from a blood sample or by apheresis or other conventional means). (It should be noted that B cells are described in this example; however, in other embodiments, other antibody-producing cells may also be used, for example, plasma cells). From the plurality of B cells, at least one B cell that produces a first antibody which associates with all or a portion of an agent of interest may be determined (e.g., identified). In some embodiments, this determining step is performed, at least in part, using a microfluidic system. For example, as described herein, a microfluidic system may be used containing a plurality of droplets, at least some of which droplets contain one (or more) B cell. In some cases, the B cells are isolated from a subject by removing blood from the subject and screening the blood to find B cells. For instance, cells from the blood may be contained within a plurality of droplets (e.g., such that each droplet has, on the average, one cell). As another example, a plurality of B cells in droplets can be cultured (e.g., within the droplets) to allow production or secretion of antibodies, and those that do produce antibodies can be separated from those that do not produce antibodies, if desired.

As discussed herein, B cells that produce antibodies that bind to or otherwise favorably interact with the agent of interest (and the droplets that contain these B cells) can be identified and/or separated from B cells that do not produce these particular antibodies. This process may involve the use of one or more signaling entities, as described herein.

For B cells that produce a first antibody which associates with all or a portion of an agent of interest, the nucleic acid encoding for the production of the first antibody may be extracted. For example, the sequence of that cell's antibody heavy (VH) and/or light (VL) chains can be extracted. In some embodiments, this extraction is performed by rupturing the cell without breaking the droplet. In some cases, however, the droplet can be broken during the extraction process.

The DNA from the cell may be sequenced using any suitable technique known to those of ordinary skill in the art. Examples of DNA sequencing techniques include, but are not limited to, PCR (polymerase chain reaction), “sequencing by synthesis” techniques (e.g., using DNA synthesis by DNA polymerase to identify the bases present in the complementary DNA molecule), “sequencing by ligation” (e.g., using DNA ligases), “sequencing by hybridization” (using DNA microarrays), nanopore sequencing techniques, or the like. Optionally, the extracted nucleic acid sequence may be amplified, duplicated, or expanded by PCR, rolling circle replication or equivalent techniques.

In one set of embodiments, the droplets are used in combination with PCR. For example, in some cases a normal PCR mixture is divided between the aqueous droplets of a water/oil emulsion such that there is, in most cases, not more than one template DNA molecule per droplet. The emulsion then may be thermo-cycled and each of the template DNA molecules may be amplified in a separate droplet. However, in other embodiments, the droplets are first broken, then the nucleic acid sequenced using PCR or other sequencing techniques known to those of ordinary skill in the art.

The extracted (or duplicated) nucleic acid sequence may be inserted into a host cell (e.g., an immortalized cell such as a CHO cell, etc.) that can subsequently express the antibody. This cell can then be used to produce a second antibody, and the cell may be optionally cloned or otherwise cultured for further antibody production. Examples of methods of transfecting a cell with a nucleotide sequence are well-known to those of ordinary skill in the art, and are described in greater detail below.

However, it should be understood that in some cases, no host cell is needed. For instance, the antibody or other species may be produced in a cell or in a cell-free expression system. Cell-free translation systems will often comprise a cell extract, typically from bacteria (Zubay, G. (1973) Annu Rev. Genet., 7, 267-287; Zubay, G. Methods Enzymol., 65, 856-877; Lesley, S. A. (1991) J. Biol. Chem. 266, 2632-2638; Lesley, S. A. et al. (1995) Methods Mol. Biol. 37, 265-278), rabbit reticulocye (Pelham and Jackson, (1976), Eur. J. Biochem, 67, 247-256), wheat germ (Anderson, C. W. et al. (1983) Methods Enzymol, 101, 635-644), etc., or are partially recombinant, cell-free, protein-synthesis systems reconstituted from elements of systems such as the Escherichia coli translation system (Shimizu, Y. et al. (2001) Nat. Biotechnol. 19, 751-755). Commercial cell-free translation systems are available from a number of suppliers including Invitrogen, Roche, Novagen, or Promega.

In some cases, the first antibody produced by the B cell is the same as the second antibody produced by the antibody-producing cell, since the nucleic acid inserted into the antibody-producing cell encodes for the production of the first antibody. However, in some instances, misfolding or other events (e.g., different types of posttranslational modifications) can occur during antibody production. In some cases, such differences may arise from different cell types, and/or different cell species. This may result in the formation of, for example, a second antibody that has a different structure than the first antibody, but has the same activity as the first antibody. Alternatively, a second antibody that has a different structure and different activity than the first antibody may be produced.

In order to verify the binding and/or activity of the second antibody, a second antibody or antibody-producing cell that produces a “hit” may be tested as described herein and/or by conventional tests. Furthermore, in some cases, the second antibody may be further optimized, e.g., by directed evolution, and/or further screened to produce an antibody (e.g., a third antibody) having more optimal activity or binding.

As an example of directed evolution techniques, a nucleotide sequence encoding an antibody or a fragment of an antibody may be subjected to various mutation, expressed in cells, then the antibodies having desired characteristics or features (e.g., determined using assays as discussed herein) selected (for instance, using techniques such as those discussed herein, or other techniques) and subjected to further mutations. Mutations can be introduced by a variety of techniques in vivo, for instance, using mutator strains of bacteria such as E. coli mutD5, or using the antibody hypermutation system of B-lymphocytes. Random mutations can also be introduced both in vivo and in vitro by chemical mutagens, or ionising or UV irradiation, or incorporation of mutagenic base analogs. Random mutations can also be introduced into genes in vitro during polymerization for example by using error-prone polymerases. Further diversification can be introduced by using homologous recombination either in vivo or in vitro.

The second (or third) antibody or a derivative thereof may also be administered, in some embodiments, to a subject in a therapeutic amount (e.g., “passive immunization”). This may allow, for instance, an amplification of an immune response of the subject from where the original sample was taken, and/or conveyance of some of the immune response of the subject who provided the sample to other subjects. In some embodiments, the second (or third) antibody or a derivative thereof can be used in combination with other therapies or used to direct reagents to work against the original “agent”; it may also be used, in some cases as a diagnostic reagent when included in a measurement system that can assay antibody binding or activity against a sample.

In administering the antibodies to a subject, dosing amounts, dosing schedules, routes of administration, and the like may be selected so as to affect known activities of these compositions. Dosages may be estimated based on the results of experimental models, optionally in combination with the results of assays of compositions of the present invention. Dosage may be adjusted appropriately to achieve desired drug levels, local or systemic, depending upon the mode of administration. The doses may be given in one or several administrations per day. In the event that the response of a particular subject is insufficient at such doses, even higher doses (or effectively higher doses by a different, more localized delivery route) may be employed to the extent that subject tolerance permits. Multiple doses per day are also contemplated in some cases to achieve appropriate systemic levels of the composition within the subject or within the active site of the subject.

Administration of the antibodies (or other species) may be accomplished by any medically acceptable method which allows it to reach its target. The particular mode selected will depend of course, upon factors such as those previously described, for example, the particular composition, the severity of the state of the subject being treated, the dosage required for therapeutic efficacy, etc. As used herein, a “medically acceptable” mode of treatment is a mode able to produce effective levels of the composition within the subject without causing clinically unacceptable adverse effects.

Any medically acceptable method may be used for administration to the subject. The administration may be localized (i.e., to a particular region, physiological system, tissue, organ, or cell type) or systemic, depending on the condition to be treated. For example, the composition may be administered orally, vaginally, rectally, buccally, pulmonary, topically, nasally, transdermally, through parenteral injection or implantation, via surgical administration, or any other method of administration where access to the target by the composition of the invention is achieved. Examples of parenteral modalities that can be used with the invention include intravenous, intradermal, subcutaneous, intracavity, intramuscular, intraperitoneal, epidural, or intrathecal. Examples of implantation modalities include any implantable or injectable drug delivery system. Oral administration may be preferred in some embodiments because of the convenience to the subject as well as the dosing schedule. Compositions suitable for oral administration may be presented as discrete units such as hard or soft capsules, pills, cachettes, tablets, troches, or lozenges, each containing a predetermined amount of the active compound. Other oral compositions suitable for use with the invention include solutions or suspensions in aqueous or non-aqueous liquids such as a syrup, an elixir, or an emulsion. Administration of the composition can be alone, or in combination with other therapeutic agents and/or compositions.

In certain embodiments of the invention, an antibody or other species be combined with a suitable pharmaceutically acceptable carrier, for example, as incorporated into a liposome, incorporated into a polymer release system, or suspended in a liquid, e.g., in a dissolved form or a colloidal form. In general, pharmaceutically acceptable carriers suitable for use in the invention are well-known to those of ordinary skill in the art. As used herein, a “pharmaceutically acceptable carrier” refers to a non-toxic material that does not significantly interfere with the effectiveness of the biological activity of the active compound(s) to be administered, but is used as a formulation ingredient, for example, to stabilize or protect the active compound(s) within the composition before use. The term “carrier” denotes an organic or inorganic ingredient, which may be natural or synthetic, with which one or more active compounds of the invention are combined to facilitate the application of the composition. The carrier may be co-mingled or otherwise mixed with one or more active compounds of the present invention, and with each other, in a manner such that there is no interaction which would substantially impair the desired pharmaceutical efficacy. The carrier may be either soluble or insoluble, depending on the application. Examples of well-known carriers include glass, polystyrene, polypropylene, polyethylene, dextran, nylon, amylase, natural and modified cellulose, polyacrylamide, agarose and magnetite. The nature of the carrier can be either soluble or insoluble. Those skilled in the art will know of other suitable carriers, or will be able to ascertain such, using only routine experimentation.

In some embodiments, the pharmaceutically acceptable carriers of the present invention may include formulation ingredients such as salts, carriers, buffering agents, emulsifiers, diluents, excipients, chelating agents, fillers, drying agents, antioxidants, antimicrobials, preservatives, binding agents, bulking agents, silicas, solubilizers, or stabilizers that may be used with the active compound. For example, if the formulation is a liquid, the carrier may be a solvent, partial solvent, or non-solvent, and may be aqueous or organically based. Examples of suitable formulation ingredients include diluents such as calcium carbonate, sodium carbonate, lactose, kaolin, calcium phosphate, or sodium phosphate; granulating and disintegrating agents such as corn starch or algenic acid; binding agents such as starch, gelatin or acacia; lubricating agents such as magnesium stearate, stearic acid, or talc; time-delay materials such as glycerol monostearate or glycerol distearate; suspending agents such as sodium carboxymethylcellulose, methylcellulose, hydroxypropylmethylcellulose, sodium alginate, polyvinylpyrrolidone; dispersing or wetting agents such as lecithin or other naturally-occurring phosphatides; thickening agents such as cetyl alcohol or beeswax; buffering agents such as acetic acid and salts thereof, citric acid and salts thereof, boric acid and salts thereof, or phosphoric acid and salts thereof; or preservatives such as benzalkonium chloride, chlorobutanol, parabens, or thimerosal. Suitable carrier concentrations can be determined by those of ordinary skill in the art, using no more than routine experimentation. The compositions of the invention may be formulated into preparations in solid, semi-solid, liquid or gaseous forms such as tablets, capsules, elixirs, powders, granules, ointments, solutions, depositories, inhalants or injectables. Those of ordinary skill in the art will know of other suitable formulation ingredients, or will be able to ascertain such, using only routine experimentation.

Preparations include sterile aqueous or nonaqueous solutions, suspensions and emulsions, which can be isotonic with the blood of the subject in certain embodiments. Examples of nonaqueous solvents are polypropylene glycol, polyethylene glycol, vegetable oil such as olive oil, sesame oil, coconut oil, arachis oil, peanut oil, mineral oil, injectable organic esters such as ethyl oleate, or fixed oils including synthetic mono or di-glycerides. Aqueous carriers include water, alcoholic/aqueous solutions, emulsions or suspensions, including saline and buffered media. Parenteral vehicles include sodium chloride solution, 1,3-butandiol, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. Preservatives and other additives may also be present such as, for example, antimicrobials, antioxidants, chelating agents and inert gases and the like. Those of skill in the art can readily determine the various parameters for preparing and formulating the compositions of the invention without resort to undue experimentation.

In some embodiments, the present invention includes the step of bringing an antibody or other species into association or contact with a suitable carrier, which may constitute one or more accessory ingredients. The final compositions may be prepared by any suitable technique, for example, by uniformly and intimately bringing the composition into association with a liquid carrier, a finely divided solid carrier or both, optionally with one or more formulation ingredients as previously described, and then, if necessary, shaping the product.

In some embodiments, the antibody or other species may be present as a pharmaceutically acceptable salt. The term “pharmaceutically acceptable salts” includes salts of the composition, prepared in combination with, for example, acids or bases, depending on the particular compounds found within the composition and the treatment modality desired. Pharmaceutically acceptable salts can be prepared as alkaline metal salts, such as lithium, sodium, or potassium salts; or as alkaline earth salts, such as beryllium, magnesium or calcium salts. Examples of suitable bases that may be used to form salts include ammonium, or mineral bases such as sodium hydroxide, lithium hydroxide, potassium hydroxide, calcium hydroxide, magnesium hydroxide, and the like. Examples of suitable acids that may be used to form salts include inorganic or mineral acids such as hydrochloric, hydrobromic, hydroiodic, hydrofluoric, nitric, carbonic, monohydrogencarbonic, phosphoric, monohydrogenphosphoric, dihydrogenphosphoric, sulfuric, monohydrogensulfuric, phosphorous acids and the like. Other suitable acids include organic acids, for example, acetic, propionic, isobutyric, maleic, malonic, benzoic, succinic, suberic, fumaric, mandelic, phthalic, benzenesulfonic, p-tolylsulfonic, citric, tartaric, methanesulfonic, glucuronic, galacturonic, salicylic, formic, naphthalene-2-sulfonic, and the like. Still other suitable acids include amino acids such as arginate, aspartate, glutamate, and the like.

As mentioned, in some embodiments of the invention, a nucleotide sequence encoding an antibody or a portion of antibody (e.g., a light chain or a heavy chain) may be delivered into a cell, for example, to be expressed by the cell. The cell may be, for example, a CHO cell, a bacteria, an immortal cell, etc. For instance, an antibody-producing cell may be determined as discussed herein, and its DNA sequenced using techniques known to those of ordinary skill in the art. In some cases, portions of genetic sequence used to produce antibodies or antibody fragments may be identified, and the portions transfected or inserted into another, host cell that causes the cell to produce the target nucleotide sequence (for example, a gene that causes the cell to produce an antibody). Any method or delivery system may be used for the delivery and/or transfection of the nucleic acid in the cell, for example, but not limited to particle gun technology, colloidal dispersion systems, electroporation, vectors, and the like.

In its broadest sense, a “delivery system,” as used herein, is any vehicle capable of facilitating delivery of a nucleic acid (or nucleic acid complex) to a cell and/or uptake of the nucleic acid by the cell. Other example delivery systems that can be used to facilitate uptake by a cell of the nucleic acid include calcium phosphate and other chemical mediators of intracellular transport, microinjection compositions, and homologous recombination compositions (e.g., for integrating a gene into a preselected location within the chromosome of the cell).

The term “transfection,” as used herein, refers to the introduction of a nucleic acid into a cell. Transfection may be accomplished by a variety of means known to the art. Such methods include, but are not limited to, particle bombardment mediated transformation (e.g., Finer et al., Curr. Top. Microbiol. Immunol., 240:59 (1999)), viral infection (e.g., Porta and Lomonossoff, Mol. Biotechnol. 5:209 (1996)), microinjection, electroporation, and liposome injection. Standard molecular biology techniques are common in the art (See e.g., Sambrook, J. et al., Molecular Cloning: A Laboratory Manual, 2^{nd} ed., Cold Spring Harbor Laboratory Press, New York (1989)).

For instance, in one set of embodiments, genetic material may be introduced into a cell using particle gun technology, also called microprojectile or microparticle bombardment, which involves the use of high velocity accelerated particles. In this method, small, high-density particles (microprojectiles) are accelerated to high velocity in conjunction with a larger, powder-fired macroprojectile in a particle gun apparatus. The microprojectiles have sufficient momentum to penetrate cell walls and membranes, and can carry DNA or other nucleic acids into the interiors of bombarded cells. It has been demonstrated that such microprojectiles can enter cells without causing death of the cells, and that they can effectively deliver foreign genetic material into intact tissue.

In another set of embodiments, a colloidal dispersion system may be used to facilitate delivery of the nucleic acid (or nucleic acid complex) into the cell. As used herein, a “colloidal dispersion system” refers to a natural or synthetic molecule, other than those derived from bacteriological or viral sources, capable of delivering to and releasing the nucleic acid to the cell. Colloidal dispersion systems include, but are not limited to, macromolecular complexes, beads, and lipid-based systems including oil-in-water emulsions, micelles, mixed micelles, and liposomes. One example of a colloidal dispersion system is a liposome. Liposomes are artificial membrane vessels. It has been shown that large unilamellar vessels (“LUV”), which range in size from 0.2 to 4.0 microns can encapsulate large macromolecules within the aqueous interior and these macromolecules can be delivered to cells in a biologically active form (Fraley, et al., Trends Biochem. Sci., 6:77 (1981)).

Lipid formulations for transfection and/or intracellular delivery of nucleic acids are commercially available, for instance, from QIAGEN, for example as EFFECTENE® (a non-liposomal lipid with a special DNA condensing enhancer) and SUPER-FELT® (a novel acting dendrimeric technology) as well as Gibco BRL, for example, as LIPOFECTIN® and LIPOFECTACE®, which are formed of cationic lipids such as N-[1-(2,3-dioleyloxy)-propyl]-N,N,N-trimethylammonium chloride (DOTMA) and dimethyl dioctadecylammonium bromide (DDAB). Methods for making liposomes are well known in the art and have been described in many publications. Liposomes were described in a review article by Gregoriadis, G., Trends in Biotechnology 3:235-241 (1985), which is hereby incorporated by reference.

Electroporation may be used, in another set of embodiments, to deliver a nucleic acid (or nucleic acid complex) to the cell. Electroporation, as used herein, is the application of electricity to a cell in such a way as to cause delivery of the nucleic acid into the cell without killing the cell. Typically, electroporation includes the application of one or more electrical voltage “pulses” having relatively short durations (usually less than 1 second, and often on the scale of milliseconds or microseconds) to a media containing the cells. The electrical pulses typically facilitate the non-lethal transport of extracellular nucleic acids into the cells. The exact electroporation protocols (such as the number of pulses, duration of pulses, pulse waveforms, etc.), will depend on factors such as the cell type, the cell media, the number of cells, the substance(s) to be delivered, etc., and can be determined by one of ordinary skill in the art.

In yet another set of embodiments, the nucleic acid may be delivered to the cell in a vector. In its broadest sense, a “vector” is any vehicle capable of facilitating the transfer of the nucleic acid to the cell such that the nucleic acid can be processed and/or expressed in the cell. Preferably, the vector transports the nucleic acid to the cells with reduced degradation, relative to the extent of degradation that would result in the absence of the vector. The vector optionally includes gene expression sequences or other components able to enhance expression of the nucleic acid within the cell. The invention also encompasses the cells transfected with these vectors. Host cells include, for instance, cells and cell lines, e.g. prokaryotic cells (e.g., E. coli) and eukaryotic cells (e.g., dendritic cells, CHO cells, COS cells, yeast expression systems, and recombinant baculovirus expression in insect cells). Other cells have been previously described.

In general, vectors useful in the invention include, but are not limited to, plasmids, phagemids, viruses, other vehicles derived from viral or bacterial sources that have been manipulated by the insertion or incorporation of the nucleotide sequence (or precursor nucleic acid) of the invention. Viral vectors useful in certain embodiments include, but are not limited to, nucleic acid sequences from the following viruses: retroviruses such as Moloney murine leukemia viruses, Harvey murine sarcoma viruses, murine mammary tumor viruses, and Rouse sarcoma viruses; adenovirus, or other adeno-associated viruses; SV40-type viruses; polyoma viruses; Epstein-Barr viruses; papilloma viruses; herpes virus; vaccinia virus; polio viruses; and RNA viruses such as retroviruses. One can readily employ other vectors not named but known to the art.

Some viral vectors can be based on non-cytopathic eukaryotic viruses in which non-essential genes have been replaced with the nucleotide sequence of interest. Non-cytopathic viruses include retroviruses, the life cycle of which involves reverse transcription of genomic viral RNA into DNA with subsequent proviral integration into host cellular DNA.

Genetically altered retroviral expression vectors may have general utility for the high-efficiency transduction of nucleic acids. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the cells with viral particles) can be found in Kriegler, M., Gene Transfer and Expression, A Laboratory Manual, W.H. Freeman Co., New York (1990) and Murry, E. J. Ed., Methods in Molecular Biology, Vol. 7, Humana Press, Inc., Cliffton, N.J. (1991), both hereby incorporated by reference.

Another example of a virus for certain applications is the adeno-associated virus, which is a double-stranded DNA virus. The adeno-associated virus can be engineered to be replication-deficient and is capable of infecting a wide range of cell types and species. The adeno-associated virus further has advantages, such as heat and lipid solvent stability; high transduction frequencies in cells of diverse lineages, including hemopoietic cells; and/or lack of superinfection inhibition, which may allow multiple series of transductions.

Another vector suitable for use with the invention is a plasmid vector. Plasmid vectors have been extensively described in the art and are well-known to those of skill in the art. See e.g., Sambrook, et al., Molecular Cloning: A Laboratory Manual, Second Edition, Cold Spring Harbor Laboratory Press, 1989. These plasmids may have a promoter compatible with the host cell, and the plasmids can express a peptide from a gene operatively encoded within the plasmid. Some commonly used plasmids include pBR322, pUC18, pUC19, pRC/CMV, SV40, and pBlueScript. Other plasmids are well-known to those of ordinary skill in the art. Additionally, plasmids may be custom-designed, for example, using restriction enzymes and ligation reactions, to remove and add specific fragments of DNA or other nucleic acids, as necessary. The present invention also includes vectors for producing nucleic acids or precursor nucleic acids containing a desired nucleotide sequence (which can, for instance, then be expressed or otherwise processed within the cell to produce antibodies). These vectors may include a sequence encoding a nucleic acid and an in vivo expression element, as further described below. In some cases, the in vivo expression element includes at least one promoter.

The nucleic acid, in one embodiment, may be operably linked to a gene expression sequence which directs the expression of the nucleic acid within the cell (e.g., to produce antibodies). The nucleic acid sequence and the gene expression sequence are said to be “operably linked” when they are covalently linked in such a way as to place the transcription of the nucleic acid sequence under the influence or control of the gene expression sequence. A “gene expression sequence,” as used herein, is any regulatory nucleotide sequence, such as a promoter sequence or promoter-enhancer combination, which facilitates the efficient transcription and translation of the nucleotide sequence to which it is operably linked. The gene expression sequence may, for example, be a eukaryotic promoter or a viral promoter, such as a constitutive or inducible promoter. Promoters and enhancers consist of short arrays of DNA sequences that interact specifically with cellular proteins involved in transcription, for instance, as discussed in Maniatis, T. et al., Science 236:1237 (1987), incorporated herein by reference. Promoter and enhancer elements have been isolated from a variety of eukaryotic sources including genes in plant, yeast, insect and mammalian cells and viruses (analogous control elements, i.e., promoters, are also found in prokaryotes).

The selection of a particular promoter and enhancer depends on what cell type is to be used and the mode of delivery. For example, a wide variety of promoters have been isolated from plants and animals, which are functional not only in the cellular source of the promoter, but also in numerous other plant and/or animal species. There are also other promoters (e.g., viral and Ti-plasmid) which can be used. For example, these promoters include promoters from the Ti-plasmid, such as the octopine synthase promoter, the nopaline synthase promoter, the mannopine synthase promoter, and promoters from other open reading frames in the T-DNA, such as ORF7, etc. Promoters isolated from plant viruses include the 35S promoter from cauliflower mosaic virus (CaMV). Promoters that have been isolated and reported for use in plants include ribulose-1,3-biphosphate carboxylase small subunit promoter, phaseolin promoter, etc.

Exemplary viral promoters which function constitutively in eukaryotic cells include, for example, promoters from the simian virus, papilloma virus, adenovirus, human immunodeficiency virus (HIV), Rous sarcoma virus, cytomegalovirus, the long terminal repeats (LTR) of Moloney leukemia virus and other retroviruses, and the thymidine kinase promoter of herpes simplex virus. Other constitutive promoters are known to those of ordinary skill in the art. The promoters useful as gene expression sequences of the invention also include inducible promoters. Inducible promoters are expressed in the presence of an inducing agent. For example, the metallothionein promoter is induced to promote transcription and translation in the presence of certain metal ions. Other inducible promoters are known to those of ordinary skill in the art.

Thus, a variety of promoters and regulatory elements may be used in the expression vectors of the present invention. For example, in some preferred embodiments an inducible promoter is used to allow control of nucleic acid expression through the presentation of external stimuli (e.g., environmentally inducible promoters). Thus, the timing and amount of nucleic acid expression may be controlled. Non-limiting examples of expression systems, promoters, inducible promoters, environmentally inducible promoters, and enhancers are described in International Patent Application Publications WO 00/12714, WO 00/11175, WO 00/12713, WO 00/03012, WO 00/03017, WO 00/01832, WO 99/50428, WO 99/46976 and U.S. Pat. Nos. 6,028,250, 5,959,176, 5,907,086, 5,898,096, 5,824,857, 5,744,334, 5,689,044, and 5,612,472 each of which is herein incorporated by reference in its entirety.

As used herein, an “expression element” can be any regulatory nucleotide sequence, such as a promoter sequence or promoter-enhancer combination, which facilitates the efficient expression of the nucleic acid. The expression element may, for example, be a mammalian or viral promoter, such as a constitutive or inducible promoter. Constitutive mammalian promoters include, but are not limited to, polymerase promoters as well as the promoters for the following genes: hypoxanthine phosphoribosyl transferase (HPTR), adenosine deaminase, pyruvate kinase, and alpha-actin. Exemplary viral promoters which function constitutively in eukaryotic cells include, for example, promoters from the simian virus, papilloma virus, adenovirus, human immunodeficiency virus (HIV), Rous sarcoma virus, cytomegalovirus, the long terminal repeats (LTR) of Moloney leukemia virus and other retroviruses, and the thymidine kinase promoter of herpes simplex virus. Other constitutive promoters are known to those of ordinary skill in the art. Promoters useful as expression elements of the invention also include inducible promoters. Inducible promoters are expressed in the presence of an inducing agent. For example, a metallothionein promoter can be induced to promote transcription in the presence of certain metal ions. Other inducible promoters are known to those of ordinary skill in the art. The in vivo expression element can include, as necessary, 5′ non-transcribing and 5′ non-translating sequences involved with the initiation of transcription, and can optionally include enhancer sequences or upstream activator sequences.

Using any gene transfer technique, such as the above-listed techniques, an expression vector harboring the nucleic acid may be transformed into a cell to achieve temporary or prolonged expression. Any suitable expression system may be used, so long as it is capable of undergoing transformation and expressing of the precursor nucleic acid in the cell. In one embodiment, a pET vector (Novagen, Madison, Wis.), or a pBI vector (Clontech, Palo Alto, Calif.) is used as the expression vector. In some embodiments an expression vector further encoding a green fluorescent protein (GFP) is used to allow simple selection of transfected cells and to monitor expression levels. Non-limiting examples of such vectors include Clontech's “Living Colors Vectors” pEYFP and pEYFP-C1.

In some cases, a selectable marker may be included with the nucleic acid being delivered. As used herein, the term “selectable marker” refers to the use of a gene that encodes an enzymatic or other detectable activity (e.g., luminescence or fluorescence) that confers the ability to grow in medium lacking what would otherwise be an essential nutrient. A selectable marker may also confer resistance to an antibiotic or drug upon the cell in which the selectable marker is expressed. Selectable markers may be “dominant” in some cases; a dominant selectable marker encodes an enzymatic or other activity (e.g., luminescence or fluorescence) that can be detected in any cell or cell line.

In one aspect, the present invention is directed to a kit. The kit may, for instance, include one or more antigen-presenting cells or other cells able to express a species. For instance, the kit may be shipped to a user. A “kit,” as used herein, typically defines a package or an assembly including one or more of the compositions of the invention, and/or other compositions associated with the invention, for example, as previously described. Each of the compositions of the kit may be provided in liquid form (e.g., in solution), or in solid form (e.g., a dried powder). In certain cases, some of the compositions may be constitutable or otherwise processable (e.g., to an active form), for example, by the addition of a suitable solvent or other species, which may or may not be provided with the kit. Examples of other compositions or components associated with the invention include, but are not limited to, solvents, surfactants, diluents, salts, buffers, emulsifiers, chelating agents, fillers, antioxidants, binding agents, bulking agents, preservatives, drying agents, antimicrobials, needles, syringes, packaging materials, tubes, bottles, flasks, beakers, dishes, fits, filters, rings, clamps, wraps, patches, containers, and the like, for example, for using, administering, modifying, assembling, storing, packaging, preparing, mixing, diluting, and/or preserving the compositions components for a particular use, for example, to a sample and/or a subject.

A kit of the invention may, in some cases, include instructions in any form that are provided in connection with the compositions of the invention in such a manner that one of ordinary skill in the art would recognize that the instructions are to be associated with the compositions of the invention. For instance, the instructions may include instructions for the use, modification, mixing, diluting, preserving, administering, assembly, storage, packaging, and/or preparation of the compositions and/or other compositions associated with the kit. In some cases, the instructions may also include instructions for the delivery and/or administration of the compositions, for example, for a particular use, e.g., to a sample and/or a subject. The instructions may be provided in any form recognizable by one of ordinary skill in the art as a suitable vehicle for containing such instructions, for example, written or published, verbal, audible (e.g., telephonic), digital, optical, visual (e.g., videotape, DVD, etc.) or electronic communications (including Internet or web-based communications), provided in any manner.

In some aspects, systems and methods of promoting one or more of the embodiments described above are provided. As used herein, “promoted” includes all methods of doing business including, but not limited to, methods of selling, advertising, assigning, licensing, contracting, instructing, educating, researching, importing, exporting, negotiating, financing, loaning, trading, vending, reselling, distributing, repairing, replacing, insuring, suing, patenting, or the like that are associated with the systems, devices, apparatuses, articles, methods, compositions, kits, etc. of the invention as discussed herein. Methods of promotion can be performed by any party including, but not limited to, personal parties, businesses (public or private), partnerships, corporations, trusts, contractual or sub-contractual agencies, educational institutions such as colleges and universities, research institutions, hospitals or other clinical institutions, governmental agencies, etc. Promotional activities may include communications of any form (e.g., written, oral, and/or electronic communications, such as, but not limited to, e-mail, telephonic, Internet, Web-based, etc.) that are clearly associated with the invention.

In one set of embodiments, the method of promotion may involve one or more instructions. As used herein, “instructions” can define a component of instructional utility (e.g., directions, guides, warnings, labels, notes, FAQs or “frequently asked questions,” etc.), and typically involve written instructions on or associated with the invention and/or with the packaging of the invention. Instructions can also include instructional communications in any form (e.g., oral, electronic, audible, digital, optical, visual, etc.), provided in any manner such that a user will clearly recognize that the instructions are to be associated with the invention, e.g., as discussed herein.

The following examples are intended to illustrate certain embodiments of the present invention, but do not exemplify the full scope of the invention.

### Example 1

One example illustrates a method for high-throughput screening of expressed proteins and polypeptides, according to one embodiment of the invention. Screening and directed evolution of functional proteins for new activities is still a considerable challenge. The vastness of the sequence space, i.e., the large number of possible permutations in even small proteins can make it difficult to conclude that all possible permutations were adequately tested by nature.

By using known recombinant DNA technologies, it is possible to create extremely large collections of genes, encoding mutants of a given protein. However, it has been difficult to create generic technologies that allow sampling of billions of different proteins.

Current methods to screen proteins and polypeptides for binding, catalytic or regulatory activities are based largely on screening in microtitre plates and robotic liquid handling. Today, robotic screening programs may process up to 100,000 assays a day (˜1 per second). The cost of high-throughput screening is substantial, e.g., greater than $100 million. Furthermore, the reagents' costs alone are typically about a dollar per assay, putting a financial ceiling on the number off assays which can be realistically performed.

The use of screening technologies which use more inexpensive equipment and further reducing test volumes below the 1-2 microliter capacity of 3,456-well plates would create both significant cost savings and would enable higher throughput. However, using microtitre plate technology, further miniaturization can meet with some difficulties: for example, evaporation becomes more significant in microliter volumes, and capillary action can cause “wicking” and bridging of liquid between wells.

One example illustrates droplet-based microfluidics for the high-throughput screening of proteins and polypeptides for binding, catalytic, or regulatory activities. FIG. 2 summarizes this method. This system is based on performing assays in aqueous microdroplets in a carrier oil (e.g., perfluorocarbon) in a microfluidic device. Each droplet, with a typical diameter of between 10-100 micrometers (other diameters are also possible), can function as an independent microreactor, but has a volume of only ˜0.5 pl to 0.5 nl (controllable by the user, depending on the size of the droplets). The volume of each assay is therefore reduced by 10³ to 10⁶-fold compared to a conventional assay in 1,536- or 3,456-well plates (typically having a capacity of 1-2 microliters per well). Furthermore, the microdroplets can be made and manipulated at a frequency of up to 10⁴ s⁻¹ (kHz), which is about 10⁴ times faster than existing high throughput screening technologies (up to 100,000 assays per day, or ˜1 s⁻¹), or more in some cases, as described herein. The small volume of the microdroplets means that even proteins expressed from single genes or single cells can be analyzed. This reduction in the assay volume should also give large cost savings.

Cells (e.g., mammalian, yeast, bacteria, etc.) can secrete a variety of molecules (e.g. proteins, peptides, antibodies, haptens) that can be screened. The target molecules to be determined can also be produced, for instance, by in vitro transcription, in vitro translation (IVT), coupled in vitro transcription and translation, etc. of genes encapsulated in droplets. A signaling entity may be used to determine the target molecules. For instance, the signaling entity may include a binding partner of a target ligand or substrate for an expressed protein attached to the surface of a microparticle.

In some cases, prior to encapsulation, the binding partner can be coupled to the surface of a bead (e.g., a polymer bead, a microgel bead, etc.). In some embodiments, an antibody may be coupled to a bead using, for example, anti-antibody antibodies, protein A, protein G, protein L, and/or antibodies against an epitope tag on the expressed antibody. Depending on the application and the particular signaling entity used, the bead can be functionalized in an appropriate way in order to couple the sensor ligand to it (e.g. biotin-streptavidin link, epoxy-, carboxyl-, amino-, hydroxyl-, hydrazide-, chloromethyl-groups for proteins). Expressed proteins can bind to the binding partner, and/or catalyze the transformation of the binding partner on the bead (substrate) into a product. In other cases, the binding partner may be used to regulate the activity of another molecule co-encapsulated in the droplet so as to cause the binding partner to be bound by a ligand or transformed into a product.

The binding of the expressed protein to the signaling entity on the bead can be detected, as this example illustrates, by coencapsulation of a fluorescently labeled antibody which binds to the expressed protein (for example via an epitope tag). Other examples of fluorescent labeling include, but are not limited to, for example, fusion to a fluorescent protein such as GFP and/or fusion to a CCPGCC (SEQ ID NO: 1) Lumio tag (Invitrogen). In some cases, the Lumio tag is reacted with Lumio Green Reagent which is As-derivatized fluorescein, which becomes fluorescent when bound to the Lumio-tagged protein. If the expressed protein does not bind to the sensor molecule, fluorescence may be relatively evenly distributed throughout the droplet. However, if the protein binds to the sensor molecule, fluorescence may be found to concentrate on the bead.

As another example, a fluorescently labeled ligand which specifically binds the product (and not the substrate) can be used, e.g. an antibody co-encapsulated in the droplet. If the expressed protein does not catalyze transformation of the sensor molecule (substrate) into product, the fluorescently labeled ligand may be relatively evenly distributed throughout the droplet. However, if the expressed protein catalyzes the transformation of the sensor molecule into product, the fluorescently labeled ligand may be found to be concentrated on the bead.

Fluorescence detection can be performed, in one embodiment, as follows. Using laser illumination and a fluorescence detector, droplets containing a fluorescent bead and those in which the fluorescence is distributed evenly throughout the droplet can be distinguished, and accordingly sorted. It is thus possible to detect and screen against multiple different target molecules by pre-preparing different sensor molecule-bead complexes, where the beads are themselves tagged. A non-limiting example of a suitable bead is a Luminex® bead. Other detection techniques that can be used involve determining binding, e.g., via a change in fluorescence polarization of a fluorescently labeled ligand when bound by the expressed protein, Förster resonance energy transfer (FRET) between the fluorescently labeled expressed protein and a fluorescently labeled, ligand, etc.

Examples of suitable systems include, but are not limited to, the screening of antibodies produced by hybridomas, human cells (e.g., human blood cells, such as B cells or plasma cells), bacteria or yeast or expressed in vitro (e.g., where the target molecule is an antibody and the signaling entity includes an antigen); or protein-protein interactions.

The method in this example is high-throughput, enabling drop production and detection on the order of 1 to 10 kHz. Other, higher speeds are also possible. In addition, the method includes a novel system for detecting, e.g., protein-antibody and protein-protein binding, in a fluidic droplet, for instance, via coupled beads or fluorescence intensity detection. Successful matches can be selected and the desired cells can be recovered alive.

Examples of applications of this example include, but are not limited to, rodent antibodies for research and diagnostics, human therapeutic antibodies, cell lines for antibody production, or technologies for the investigation of protein-protein interactions.

Another example illustrates the high-throughput expression screening of hybridomas for monoclonal antibody production. Monoclonal antibodies are a valuable biological reagent. They can be used for sensitive detection and quantification of target proteins of interest. Ideally, there would be a monoclonal antibody (or a small collection of monoclonal antibodies) for every protein encoded by a given genome. This would represent a library of roughly 20,000 distinct antibodies. However, the current procedure for the generation of high quality antibodies is tedious, taking about 5-6 months per antibody, at a cost of approximately $5,000/antibody. Typically, a mouse is immunized with a purified protein of interest. Spleens from immunized mice are then dissociated in cell culture to liberate lymphocytes. Lymphocytes are then fused to a myeloma cell line to create immortalized hybridomas, each of which generates a single antibody. The rate-limiting step in the generation of high quality antibodies, in certain cases, is selecting hybridomas that generate antibodies binding to a given protein of interest.

This example illustrates one method to accomplish this goal in a high-throughput manner. The method described in this example includes an expression screening strategy that makes use of in vitro translated proteins, antibodies from large collections of hybridomas, and microfluidic droplet technology.

A cDNA library can be subjected to in vitro transcription/translation. New in vitro translation technologies permit translation with incorporation of fluorescence amino acids so that these protein products are fluorescent. For example, in some embodiments, the CCPGCC Lumino tag (Invitrogen) can be used to make in vitro translated proteins fluorescent. Starting with a cDNA library, a large collection of droplets can be created, containing many copies of a single protein, as well as the cDNA, which serves as a barcode for the protein in the droplets. Individual hybridoma cells can be localized in the droplets, where they can secrete antibodies. To allow high-throughput selection of antibodies, hybridomas produced from a mouse can be used that have been immunized with a large number of proteins simultaneously. The secreted antibodies and hybridomas are thus contained within a single “hybridoma droplet.” Thus, “hybridoma droplets” can be created containing hybridoma cells as well as secreted antibody, or “IVT droplets” can be created containing cDNA and its fluorescent protein products. Hybridoma and IVT droplets can also be fused together in some cases.

By beginning with an entire library of hybridoma droplets, as well as an entire cDNA library, an entire library of IVT droplets can be produced. These droplets can be fused and then selected. The droplets can contain a hybridoma, which can now be expanded. The droplets also contain a cDNA barcode, which can be re-sequenced to identify the protein of interest. In this manner, hybridomas can be mapped to the proteins to which their secreted antibodies bind.

This method involves, as another example, the immunization of a mouse with a complex mixture of proteins. In addition, this method can be run in a high-throughput manner, and can allow for sufficient genome-scale production of antibodies. The method is also based on an expression screening, where a complete cDNA library is translated in vitro and screened for binding to a library of hybridoma antibodies.

### Example 2

In this example, microfluidic devices were used to encapsulate, incubate, and manipulate individual cells in picoliter aqueous drops in a carrier fluid at rates of up to several hundred Hz. In this set of embodiments, individual devices were used for each function, thereby increasing the robustness of the system and making it flexible and adaptable to a variety of cell-based assays. The small volumes of the drops enabled the concentrations of secreted molecules to rapidly attain detectable levels. The embodiments described herein showed that single hybridoma cells in 33-pL drops secreted detectable concentrations of antibodies in only 6 hours and remain fully viable.

In this example, the use of drop-based microfluidic devices to encapsulate single mammalian cells in distinct pL-sized drops to isolate them in their own microenvironment is described. Because the volume of each drop is restricted, molecules secreted by an individual cell can rapidly attain detectable concentrations. In this example, distinct microfluidic devices are used for encapsulation, incubation, manipulation, and analysis, significantly enhancing robustness and flexibility. This example demonstrates the power of these devices by encapsulating individual mouse hybridoma cells in drops, where they remain viable for several hours while secreting antibodies at a rate similar to cells in bulk. Moreover the cells can be recovered from the drops and cultured.

Microfluidic flow chambers were fabricated by soft lithography. Negative photoresist (e.g., SU-8 2025 or SU-8 2100 from Micro-Chem, Newton, Mass.) was deposited onto clean silicon wafers to a thickness of 25 μm, 40 μm, or 100 μm. The photoresist was patterned by exposure to UV light through a transparency photomask (CAD/Art Services, Bandon, Oreg.) and developed. Sylgard 184 poly(dimethylsiloxane) (PDMS) (Dow Corning, Midland, Mich.) was mixed with crosslinker (ratio 10:1), degassed thoroughly, poured onto the photoresist patterns, and cured for at least 1 hour at 65 degrees C. The PDMS replicas were peeled off the wafer and bonded to glass slides after oxygen-plasma activation of both surfaces. The microfluidic channels were treated with Aquapel (PPG Industries, Pittsburgh, Pa.) by filling the channels with the solution as received and subsequently flushing them with air prior to the experiments; this improved the wetting of the channels with fluorinated oil. Polyethylene tubing with an inner diameter of 0.38 mm and an outer diameter of 1.09 mm (Becton Dickinson, Franklin Lakes, N.J.) was used to connect the channels to syringes. Glass syringes were used to load the fluids into the devices. Flow rates were controlled by syringe pumps. Distinct devices were fabricated for encapsulation, incubation, and analysis. In some embodiments, devices for drop formation and cell encapsulation were 40 microns high with a 35-micron wide nozzle. To vary the drop size, varying nozzle widths were used with a channel height of 25 microns. Devices for cell incubation were 100 microns high, the channel width was 500 microns, and the length was 2.88 meters. Devices for analysis can include various on-chip functionalities, but in cases described in this example, require an interface between the incubation and analysis chips. This was accomplished with a nozzle to re-inject the drops into the channels. The reinjection nozzle was similar in geometry to the drop-formation nozzle, but was larger, with a 40-micron height and at least a 40-micron width, to facilitate the flow of drops into the devices. All inlet channels were equipped with patterned filters which prevented dust particles from clogging the channels downstream.

In this example, 2C6 hybridoma cells were grown. The 2C6 cells produced an anti-ovalbumin IgE (gift from Lester KobzikLester Kobitz), in Dulbecco's Modified Eagle Medium (DMEM) with 4.5 g/L glucose, L-glutamine, and sodium pyruvate (Mediatec, Inc. Herndon, Va.) supplemented with 10% (v/v) fetal bovine serum (FBS, SAFC Biosciences, Lenexa, Kans.) and 1% Penicillin/Streptomycin. The cells were split every 3 days under sterile conditions and incubated at 37° C. and 5% CO₂.

Cells were grown on culture dishes to a density of 1.2 to 2.5×10⁶ cells/mL. Prior to the experiments, cells were washed at least once and resuspended in fresh media. The cell density was adjusted to the desired value, which depended on the average density per drop and the drop size. Hybridoma cells were about 10 microns in diameter and the total volume of medium available to each cell was several times its own volume. Fluorinert FC40 fluorocarbon oil (3M, St. Paul, Minn.) was used to suspend the drops. To stabilize the drops a PFPE-PEG block-copolymer surfactant was added to the suspending oil at a concentration of 1.8% (w/w). This surfactant provided excellent drop stability against coalescence while ensuring good biocompatibility of the inner drop interface. For drop formation, the outer, carrier-oil flow rate was 300 microliters/hour and the inner, aqueous flow rate was 30 microliters/hour, leading to a drop production rate of 250 Hz. At this rate the incubation device was filled in 40 minutes. The cells were incubated by placing the whole device in a cell incubator at 37 degrees C. and 5% CO₂.

Drop formation was imaged with a high-speed Phantom V5 camera (Vision Research, Inc., Wayne, N.J.), and individual frames were analyzed to determine the number of cells per drop and associated statistics. For each dilution, images of 350 drops at each of three different points in time were collected during the course of the experiment.

Cells were recovered from collected emulsions by diluting the emulsion with 10× its fluid volume of fresh media and adding drop release reagent (RainDance Technologies, Inc., Lexington, Mass.) equivalent to 15% of its volume. The mixture was incubated for 2 minutes to allow the oil and release agent to settle. The supernatant containing the cells was transferred to a fresh vial. In separate tests of this procedure, no effect on cell viability was observed. To optimize the experimental conditions, cell viability was tested in each case using a live-dead assay. 1 micromolar calcein-AM (Invitrogen, Carlsbad, Calif., green fluorescence, live stain) and 1 micromolar ethidium-homodimer-1 (Invitrogen, red fluorescence, dead stain) in phosphate buffered saline (PBS) were used. The cells were incubated with the stains for 45 minutes at room temperature (RT) in the dark, and representative images of the sample were analyzed using fluorescence micrographs. Viability was determined from the fraction of live cells. This assay provided a means to compare viability under different experimental conditions.

The supernatant with the recovered cells was transferred into 96 well plates and incubated at 37° C. and 5% CO₂.

Expression of anti-ovalbumin antibodies in bulk and in drops was determined by a kinetic enzyme-linked immunosorbent assay (ELISA). Cells were placed on ice prior to encapsulation for 30 minutes and maintained at 4° C. while being washed 2 times to remove any remaining antibodies from the suspension and to prevent premature antibody production. The supernatant from each wash was tested for antibody content. For comparison, one reference culture treated in an identical manner as the cells used for encapsulation was placed into a culture dish at the same high density (10×10⁶ cells/mL) and incubated in bulk for 6 h at 37 degrees C. and 5% CO₂. Cells in drops were maintained at 37 degrees C. and 5% CO₂ on the incubation chip for 6 hours. Emulsions were broken and ELISAs were performed on culture supernatants after centrifugation to remove any remaining hybridomas. 50 microliters ovalbumin (Sigma, St. Louis, Mo.) (1 mg/mL) in PBS was added to separate wells of a 96-well plate (control wells contained only PBS) and incubated for at least 5 hours at room temperature. The antigen solution was removed, and the wells were washed 3 times with 1× Tris-buffered saline (TBS) containing 0.2% Tween-20 (TBST) for 5 min each. The wells were blocked with 200 μL 3% bovine serum albumin (BSA) in PBS for at least 2 hours at room temperature. The wells were then washed 3 times with TBST, incubating each step for 5 min. Culture supernatant dilutions were prepared in 3% BSA in PBS, and 50 microliters of the dilutions were added to each well and incubated for 1 hour. The wells were washed 3 times with TBST for 5 min each. The secondary rat anti-mouse antibody horseradish peroxidase (HRP) conjugate (clone 23G3, Southern Biotech, Birmingham, Ala.) was prepared in 3% BSA in PBS at 1:1000 dilution, added to the wells and incubated for 1 h at room temperature. The wells were washed 3 times with TBST for 5 min each, and 100 microliters of fresh substrate (o-phenylenediamine dihydrochloride, Pierce, Rockford, Ill.) in buffer solution is added to each well. The absorbance at 450 nm was read every 10 seconds for 10 min using the kinetic measurement mode of a plate reader. The measured signal was plotted as a function of time, and the initial slope was determined which provides a measure of the relative antibody concentration. The control signal obtained from wells with no protein was subtracted from the measured values.

Several distinct components were used for the all-microfluidic approach to single cell experiments: encapsulation, incubation, and manipulation devices, as indicated by the boxes in FIG. 6.

To illustrate the utility of this modular approach to drop based cell handling, a line of hybridoma cells which secrete anti-ovalbumin IgE antibodies was used. These hybridomas are suspension cells simplifying their handling in drops.

The cell encapsulation device used a flow focusing geometry to produce drops, as shown schematically on the left of FIG. 6a. Additional inlets can be incorporated on chip to mix reagents with the cells just before they are encapsulated, as shown schematically on the right of FIG. 6a. Three inlet channels, coming from the left, convert to form a nozzle as shown in the optical micrographs in FIGS. 6b and 6c. In both cases, the center stream contains the cell suspension while the side streams contain the oil phase. The drop volume can easily be varied between about 0.5 pL and about 1.8 mL, corresponding to spherical drops of diameter 10 microns to 150 microns. This was accomplished by matching the size of the nozzle orifice to the drop diameter and operating the device in the dripping regime. Fine tuning of the drop size for a given nozzle can be accomplished by varying the inner, aqueous flow rate or the overall flow rate; this also leads to variation in the drop production frequency. The modular nature of the device enables the nozzle dimension, and hence the drop size, to be readily changed without affecting any other components.

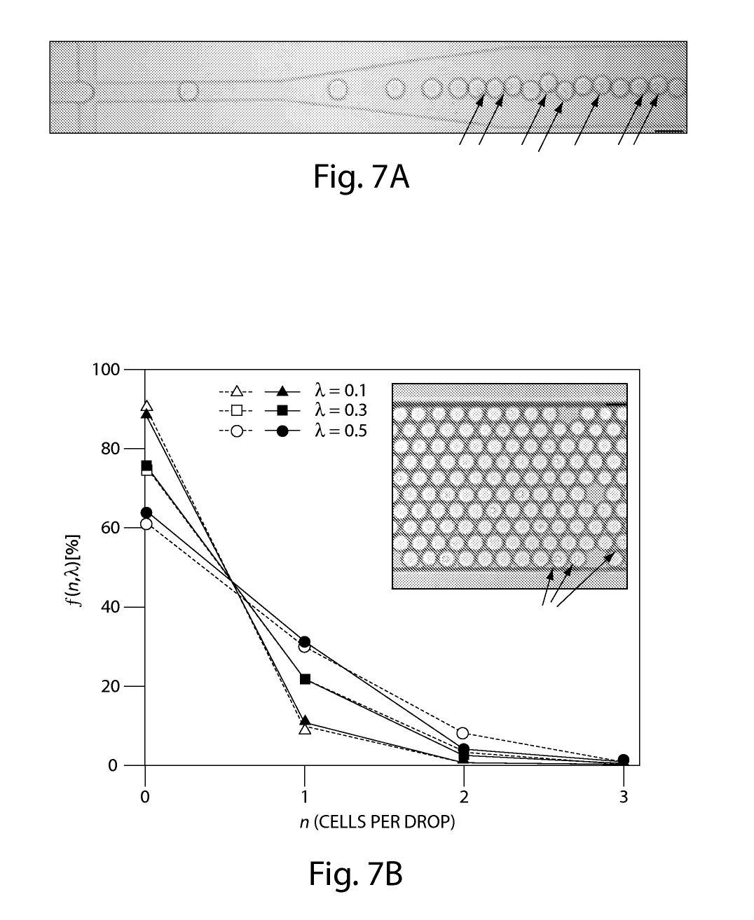

Individual syringe pumps were used to control the flow of the oil and the cell suspension. In this set of embodiments, the focus is on suspension cells; however, adherent cells can also be studied by first growing the cells on small beads and then encapsulating the beads. To prevent settling of the cells and maintain the desired density, the suspension was stirred constantly. Typically a 5 mL syringe containing 1 mL of cell suspension was used, ensuring that the depth of the volume was comparable to its height, thus enabling it to be easily mixed using a small magnetic stir bar. A convenient method of stirring the sample, while preventing clogging of the syringe, was to maintain it at a 45° upward angle and to place a stir plate on top of it. Using this scheme the encapsulation efficiency was typically approximately 70%. Account for this factor, one can reliably and reproducibly obtain the desired cell distribution in the drops. Single-cell studies require that most or all drops contain at most one cell, so that the majority of drops contain no cell at all since the encapsulation process follows Poisson statistics. Production of drops encapsulating individual cells is shown in FIG. 7a, where black arrows highlight the cell-bearing drops. The Poisson distribution for cells is given by:

$$f ( λ , n ) = \frac{{λ}^{n} {e}^{- λ}}{n !}$$

where n is the number of cells in the drop, and lambda is the average number of cells per drop; lambda can be adjusted by controlling the cell density. The distributions of cells in drops for lambda=0.1, 0.3, and 0.5 were demonstrated; these are typical values of interest for single cell experiments as they ensure that very few drops contain multiple cells. In each case, the results were in good agreement with those calculated from Poisson statistics for the values of lambda used, as shown in FIG. 7b. By using lambda=0.3, cells were observed in roughly 22% of the drops, and fewer than 4% of the drops included two or more cells. Although the number of single-cell-bearing drops was rather low, the effect was not severe in this set of embodiments, given the high production and screening rate that could be achieved with microfluidic devices.

The incubation device included a long serpentine channel with a volume of 144 microliters, enabling it to hold a large quantity of drops, as shown schematically in the top of FIG. 6d. Cell-bearing drops produced in the encapsulation device could be redirected into the incubation device by means of external tubing. Inside the device the flow rate of the carrier oil was faster than that of the drops, thereby concentrating the emulsion. Interestingly, because of their buoyancy the drops collected at the top of the channel where they formed a well-packed single layer, as shown in FIGS. 6e and f. Despite the high packing of the drops, the surfactant ensured stability, and virtually no uncontrolled coalescence was observed.

The incubation device could be detached from the encapsulation device and placed in a cell incubator or other storage container to maintain the desired temperature and gas atmosphere. By carefully maintaining the channels filled with oil, any deleterious effects of air in the channels could be avoided. The permeability of both the PDMS and the fluorocarbon carrier oil to gas enabled sufficient exchange to keep the cells at the level set by the environment; this was facilitated by their monolayer packing The water saturated atmosphere prevented evaporation of water from the drops ensuring they retained the desired size and concentration. Independent studies over long periods of time confirmed that the drop diameter shrank by less than 3.5% after 72 hours; thus, for the much shorter incubation times used in these experiments, it was determined that the shrinkage was negligible.

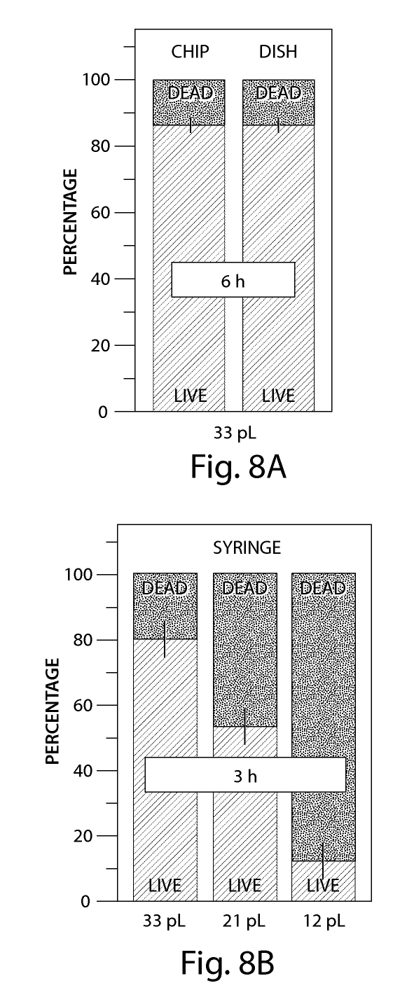

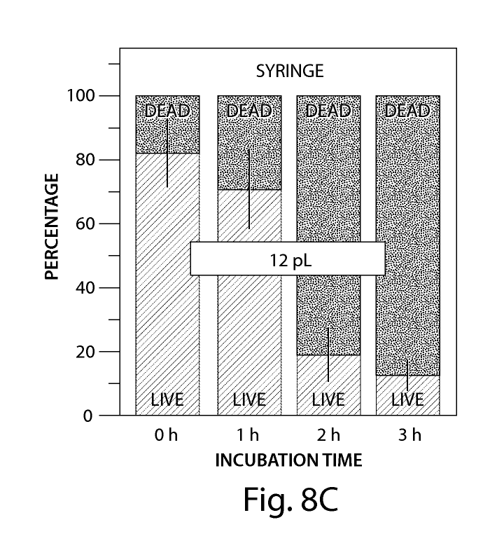

To ascertain cell viability, the emulsion was broken after incubation, the cells were recovered, and live-dead assays were performed. After incubation for a period of 6 hours, it was determined that the cells had a survival rate of approximately 85%; by comparison, an identical survival rate was found for cells incubated on culture dishes as shown in FIG. 8a. Maintaining the cells in drops and on chip for all functions greatly increased both the convenience and usefulness of these devices, and these results confirmed that this approach was feasible.

For comparison, drops were also occasionally collected directly into a syringe where the piston had been removed to allow gas exchange. In these cases, the monolayer packing of the drops was no longer maintained, even when the syringe was placed almost horizontally to increase the surface area of the fluid. As a result, cell viability was degraded, and after only 3 hours the survival rate was already only 80% as shown in FIG. 8b. These results confirmed the importance of the monolayer packing in our microfluidic incubation device for these hybridoma cells.

A confined cell-culture volume without perfusion leads to a decrease in nutrient levels and an increase in waste levels, compromising cell proliferation and growth. Therefore, the survival rate as a function of drop size was also tested. Drops with volumes of 21 pL and 12 pL showed poor results, as shown in FIG. 8b. This is clearly a function of incubation time with the survival rate decreasing dramatically with increasing time as shown in FIG. 8c. Drops of approximately 33 pL were used in the microfluidic incubation device, ensuring a good rate of cell survival for at least 6 hours. This inverse relationship between drop size and survival time is consistent with studies using other mammalian cell lines (Jurkat and HEK293T), in which microfluidic systems were used to compartmentalize single cells in larger (660 pL) drops in Fluorinert FC40 fluorocarbon oil stabilized with a PFPE-dimorpholinophosphate surfactant. In these larger drops, the cells survived and proliferated for several days before viability started to decrease.

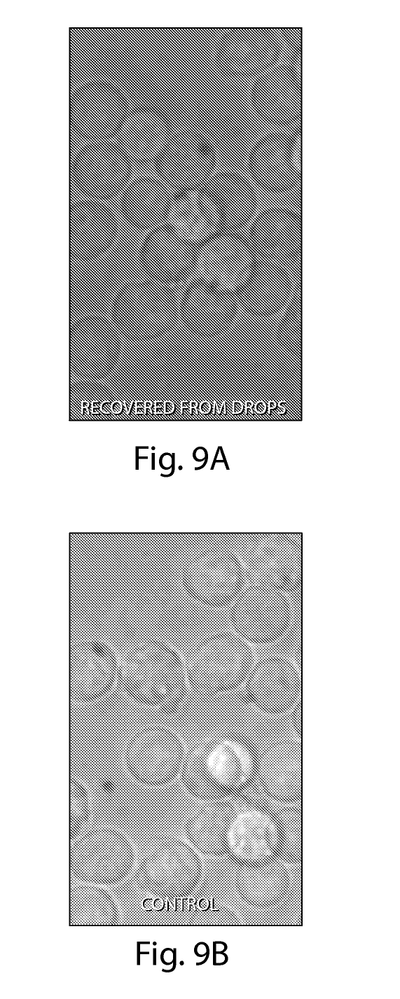

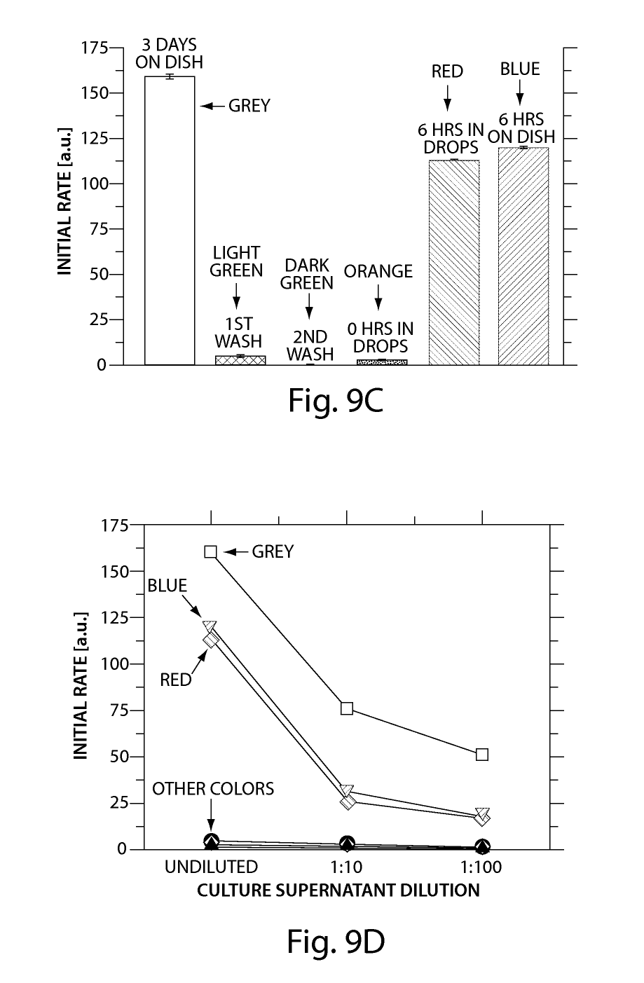

In addition to live-dead tests for cell viability, more rigorous experiments were performed to ensure that cell metabolism was not harmed by their encapsulation. This was accomplished by breaking the emulsion, recovering the cells, and recultivating them on microplates. Normal growth was observed; cells split directly from bulk were indistinguishable from those recultivated from the broken emulsion, as shown by the images, taken after 2 days growth, in FIGS. 9a and b. This set of experiments demonstrated the viability of cells encapsulated in drops and confirmed that new cell lines could, in principle, be established from encapsulated cells.

It was also ascertained that the production of antibodies was not hindered by the confinement of the hybridomas in the small volume of the drops. To prepare the hybridomas for this test, cells were provided at a density of about 2×10⁶ cells/mL, and the cells were grown for 3 days, at which time the density had increased to about 8×10⁶ cells/mL. The concentration of antibody in the supernatant was measured with an ELISA, as shown in FIG. 9c (grey). The cells were washed with fresh media twice, checking to ensure that the antibody concentration in the supernatant had decreased to a negligible value, as shown in FIG. 9c (green). The density was adjusted to 10×10⁶ cells/mL, and the cells were encapsulated. A portion of the emulsion was immediately broken to ensure that there was very little antibody production during the encapsulation process, as shown in FIG. 9c (orange). The remaining drops were incubated for 6 hours on the incubation device, and the emulsion was broken. The antibody concentration increased significantly as shown in FIG. 9c (red). As a control, the measured results were compared with those obtained from cells cultured on a dish for 6 hours at the same initial density (10×10⁶ cells/mL). Nearly identical concentrations were measured, as shown in FIG. 9c (blue). Assuming a typical rate of immunoglobulin secretion by hybridomas of 5,000 molecules/s, it was estimated that the antibody concentration in the supernatant was about 10¹⁵ molecules/mL after 6 hours. All of the ELISA measurements were performed in a regime where the signal was not saturated by performing additional experiments at ten-fold and one-hundred-fold dilutions. The measured relative concentrations decreased proportionately, verifying the consistency of the results, as shown in FIG. 9d. This confirmed that the cells were viable and that the metabolism of the encapsulated hybridoma cells was not degraded by their confinement. It also highlighted a unique feature of these drop-based microfluidic devices: the ability to rapidly attain high concentrations of secreted molecules in the confined volumes of the drops.

After on-chip incubation, further analysis of the cells and the drop contents was performed with the analysis device. This required transferring the emulsion from the incubation device to the analysis device. A syringe pump was connected by external tubing to the inlet of the incubation device and carrier fluid was used to drive the emulsion through additional external tubing, connecting it to the analysis chip. A flow-focusing geometry was used at the inlet of the analysis chip, with the auxiliary oil channels adjusting the spacing between the drops as shown in FIGS. 6g and 6h. This leads to a uniform flow of drops, which can then be run into other modules fabricated on the analysis device. Potential examples include drop merging, splitting, detecting, and/or sorting, depending on the assay desired. Alternatively, drops can be loaded onto a microfluidic device designed to store ordered arrays of drops, shown schematically in the bottom of FIG. 6d. This allows individual drops to be monitored, as shown in FIG. 6i, enabling time-resolved single-cell analysis.

The drop-based microfluidic system presented in this example was a modular, and therefore a highly flexible, system which combined distinct devices to encapsulate, incubate, and manipulate single cells in small drops (≦33 pL), enabling the concentrations of secreted molecules to rapidly attain detectable levels. The advantage of the modular concept is its flexibility, allowing adjustment to specific experimental requirements. The components here were placed on physically separate chips which were connected by means of external tubing. Thus components can be exchanged to address the different experimental demands encountered when varying assays. Moreover, dysfunctional chips can be replaced, mitigating problems caused by clogging or leakage.

It was shown in this example that antibody production, cell survival, and proliferation upon recovery were ensured despite the encapsulation in the confined geometry of the drops. These represent important preconditions for single cell experiments, such as screening for monoclonal antibodies, using drop-based microfluidics. Indeed, the small volume of the drops means that a single hybridoma cell in a drop secreted detectable concentrations of antibodies in only 6 hours, at least in some cases. The modular design of the devices also allowed for adjustment to many other functional single cell assays where statistical information from large populations of individual cells can be collected while each cell is isolated in its own microenvironment. This can thus separate the encapsulation, incubation, analysis, and sorting steps of assays. For example, drops containing other reagents or elements of a library could be merged with the cell-bearing drops prior to incubation or to sorting.

### Example 3

This example describes two complementary droplet-based microfluidic platforms which allowed fully viable human cells to be recovered with high yield after several days in microcompartments. The volume of each microcompartment can be over 1,000-fold smaller than the smallest volumes utilizable in microtiter-plate based assays, and single, or multiple human cells, as well as multicellular organisms such as C. elegans, can be compartmentalized and replicate in these systems. To show the utility of this approach for cell-based assays, automated fluorescence-based analysis of single cells in individual compartments after 16 hours of incubation was also demonstrated.

The goal of this set of examples was to set up microfluidic platforms for high-throughput cell-based assays. Hence, the technology should allow a) Encapsulation of a pre-defined number of cells per microcompartment (with the option of encapsulating single cells being highly desirable), b) Storage of the compartmentalized samples within a CO₂-incubator, and c) Recovery of the cells from the compartments in a way that does not abolish cell viability.

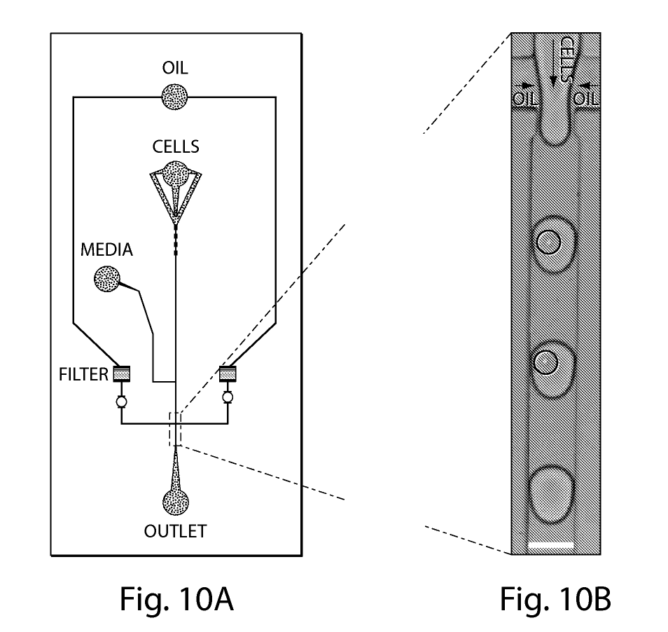

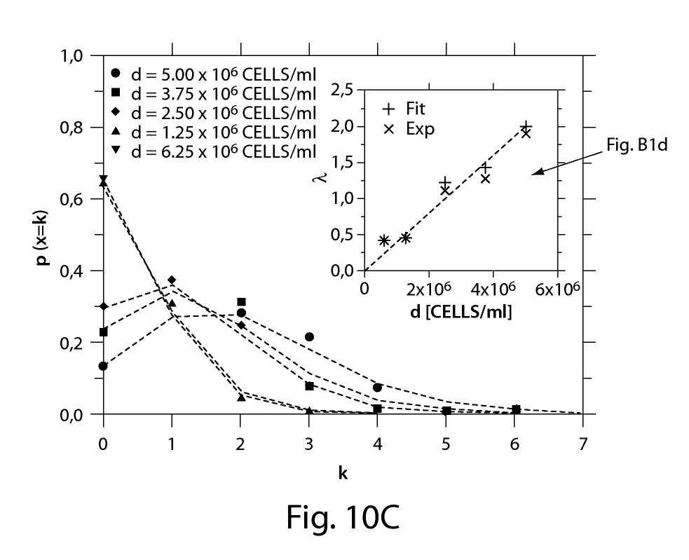

The encapsulation step (FIGS. 10A and 10B) was performed on a PDMS chip in which drops of 660 pL volume (corresponding to a spherical diameter of 100 μm±1.7%) were created from a continuous aqueous phase by “flow-focusing” using a perfluorinated carrier oil (Anna et al., 2003). Perfluorocarbon oils are well-suited for this purpose, since they are compatible with PDMS devices, immiscible with water, transparent (allowing optical readout procedures), and have been shown to facilitate respiratory gas-delivery to both prokaryotic and eukaryotic cells in culture. The number of cells per droplet was controlled using on-chip dilution of the cells to regulate the cell density (FIG. 10C). A culture of Jurkat cells, with an initial density of 5×10⁶ cells/ml, was brought together with a stream of sterile medium by co-flow immediately before drop formation and the relative flow rates of the cell suspension and the medium were changed, while keeping the sum of the two flow rates constant. The number of cells per drop (k) was in good agreement with a Poisson distribution, and high cell densities at the nozzle (≧2.5×10⁶ cells/ml) made the encapsulation of multiple cells per drop highly likely (p≧30%). In contrast, cell densities of 1.25×10⁶ cells/ml and below resulted in low probabilities (p≦7%) for the encapsulation of more than one cell per drop (while increasing the probability of finding drops without any cells inside). At the same time, the average number of cells per drop (lambda) decreased from approximately two (at 5×10⁶ cells/ml) to far below one (at ≦1.25×10⁶ cells/ml). Hence, the number of cells per drop can easily be regulated, even allowing the compartmentalization of single cells.

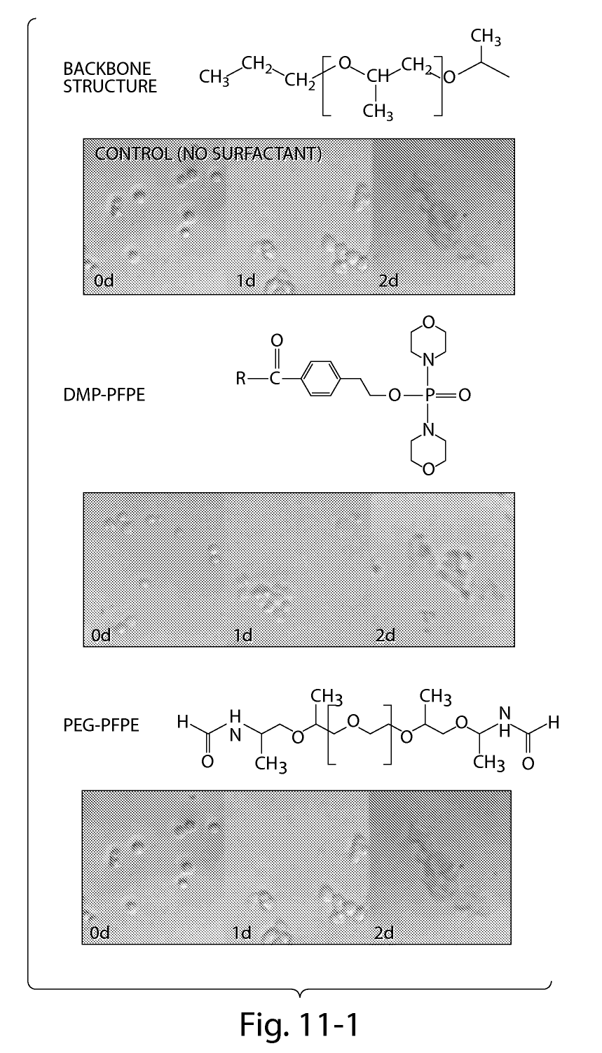

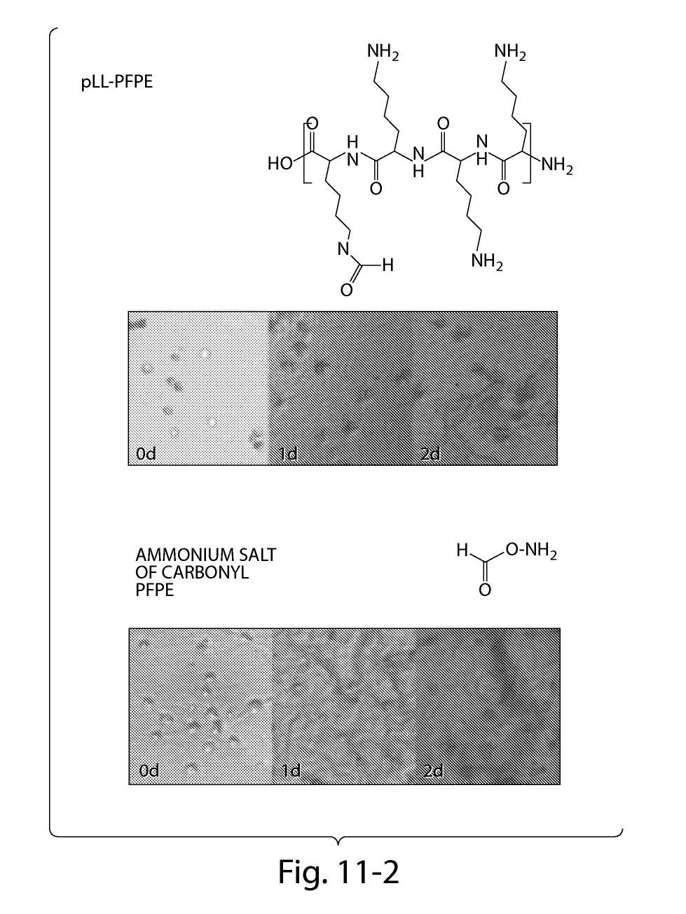

The generation of stable drops required the use of a surfactant decreasing the surface tension which, for the encapsulation of cells, also has to be biocompatible. For this reason, several perfluoropolyether-derived surfactants (PFPE surfactants) were synthesized, and their effect on long-term cell survival (FIG. 11) was tested. The surfactants differed solely in their hydrophilic head groups, which should be the only part of the molecule in contact with the encapsulated cells. The common perfluorinated tail should be dissolved in the carrier oil and thus be oriented away from the cells. To analyze the biocompatibility, HEK293T cells were seeded on top of a perfluorocarbon oil layer in the presence (0.5% w/w) and absence of different surfactants. While in the absence of any surfactant the cells retained an intact morphology and even proliferated, the ammonium salt of carboxy-PFPE (Johnston et al., 1996) and poly-L-lysine-PFPE (PLL-PFPE) mediated cell lysis. However, polyethyleneglycol-PFPE (PEG-PFPE) and dimorpholinophosphate-PFPE (DMP-PFPE) showed good biocompatibility, did not affect the integrity of the cellular membrane, and allowed cell proliferation. Since DMP-PFPE generated more stable emulsions than PEG-PFPE (data not shown), it was used for all further experiments.

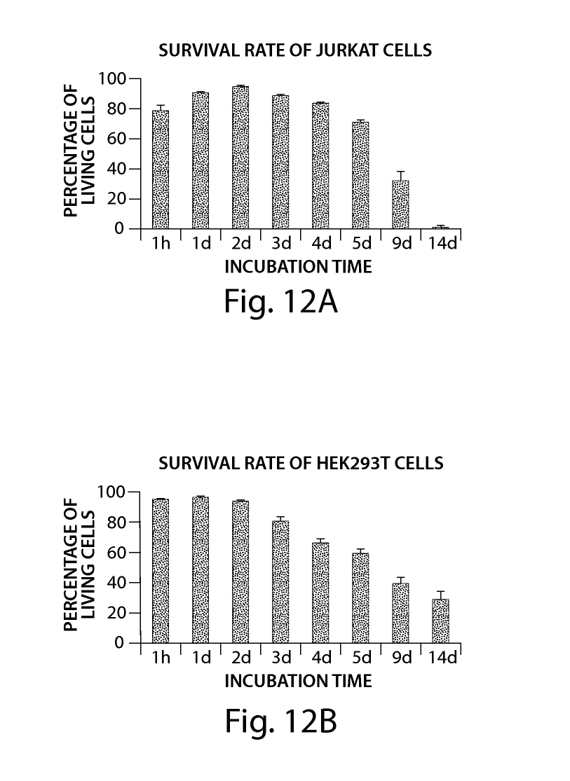

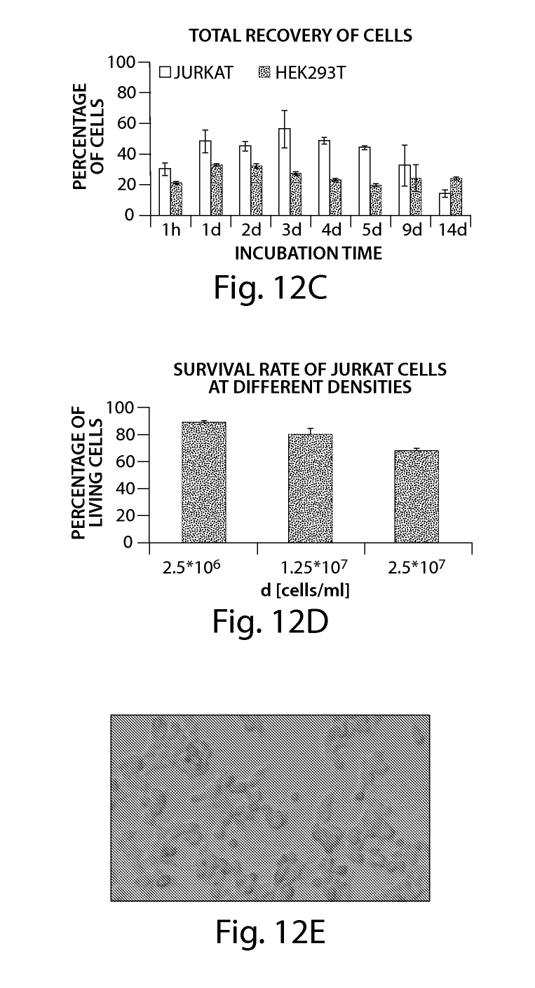

As the next step, procedures allowing the recovery of encapsulated cells had to be established. Addition of 15% (v/v) Emulsion Destabilizer A104 (RainDance Technologies) to the emulsions mediated reliable breaking without obvious impact on cell viability. This allowed the determination of the survival rates of suspension (Jurkat) and adherent cells (HEK293T) for different incubation times within drops. For this purpose, cells were encapsulated at a density corresponding to an average of less than one cell per 660 pl drop (1.25×10⁶ cells/ml at the nozzle resulting in a lambda value of about 0.55 and single cells in approximately 31.7% of all drops) and collected the resulting emulsions in 15 ml centrifugation tubes. After different incubation times at 37 degrees C. within a CO₂ incubator, the emulsions were broken and the cells were treated with a live/dead stain to determine the survival rate and the total number (live and dead) of recovered cells (FIGS. 12A and 12C). During the first four days, the fraction of recovered viable Jurkat cells did not change significantly and was always in excess of 79%. Then the percentage of live cells decreased from 71% after 5 days, to 32% after six days, and finally to 1% after 14 days of encapsulation. The total number of recovered cells divided by the number of initially encapsulated cells (equal to the aqueous flow rate multiplied by the injection time multiplied by the cell density at the nozzle) was defined as the recovery rate and increased from 29% after one hour to more than 55% after two days. This indicates some degree of proliferation within the drops, also supported by the fact that after 24 hours the percentage of dead cells was lower than after 1 hour. During further incubation within drops the recovery rates slowly decreased to just 14% after 14 days. This decrease can be explained by the fact that dead cells ultimately disintegrate (after several days) and thus cannot be stained anymore. This effect is well known and has been analyzed in detail for bacterial cells. However, early time-points and the live stain are not affected by this phenomenon. When repeating the experiments with adherent HEK293T cells, similar results were obtained (FIGS. 12B and 12C). During the first two days, the fraction of recovered viable cells remained constant at more than 90% before slowly decreasing to 58% after five days and 39% after nine days. Finally, after 14 days of encapsulation, 28% of the recovered cells were still alive. The total recovery rate increased slightly from 20% after 1 hour to more than 32% after two days. During further incubation within drops the recovery rates slowly decreased to 23% after 14 days. Not wishing to be bound by any theory, the longer cell survival compared to Jurkat cells may be due to slower proliferation resulting in slower consumption of the available nutrition. Recovered cells could also be recultivated (instead of stained) after incubation for two days within droplets, resulting in normally proliferating cells (FIG. 12E).

In a further experiment, the effect of the cell density on survival rates was assessed. For this purpose five- and ten-fold higher densities of Jurkat cells were used compared to the amounts used initially. Comparison of the cell survival after three days showed that the cell density was inversely correlated with the survival rate (FIG. 12D). While almost 90% viable cells were recovered using the initial cell density, only 80% and 68% survived for the five- and ten-fold increased cell density, respectively. Not wishing to be bound by any theory, insufficient gas exchange likely did not contribute to this effect since equally dense cultures in ordinary tissue culture flasks did not survive longer: using a density equal to one cell in a 660 pl drop (˜1.5×10⁶ cells/ml) the number of viable Jurkat cells remained above 87% for the first two days before decreasing to 51% after four days and no surviving cells after 9 days (data not shown). Therefore the encapsulated cells may have died due to the lack of nutrition or the accumulation of toxic metabolites rather than because of compartmentalization-specific factors such as the oil and surfactant.

In parallel to encapsulating cells into aqueous drops of a water-in-oil emulsion, a system was established in which aqueous plugs spaced by immiscible oil within a piece of tubing served as a culture vessel. This approach allowed the generation of aqueous microcompartments big enough to host small cell populations and even multicellular organisms. This cannot be achieved by simply increasing the drop size of a given emulsion. First, the maximum size of a drop generated on a microfluidic chip is limited by the channel dimensions. Second, as the size of the drops increases they become less stable resulting in uncontrolled sample coalescence. These problems can be circumvented by alternately aspirating aqueous plugs and immiscible oil into a holding cartridge (e.g. a capillary or a piece of tubing). This approach was used to encapsulate several thousand cells into single microcompartments.

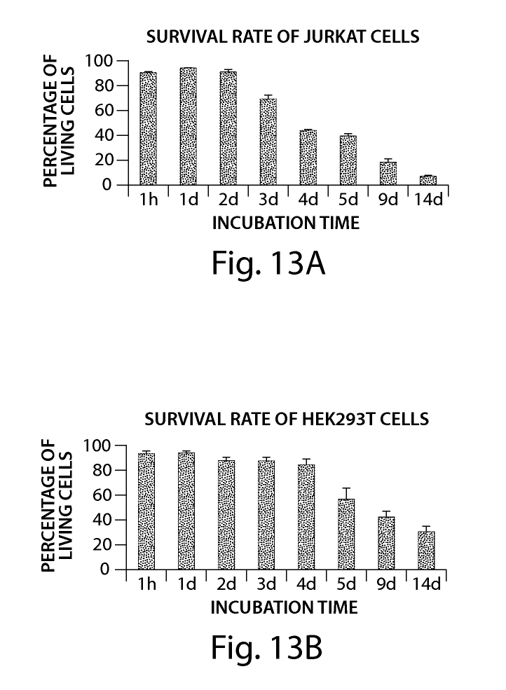

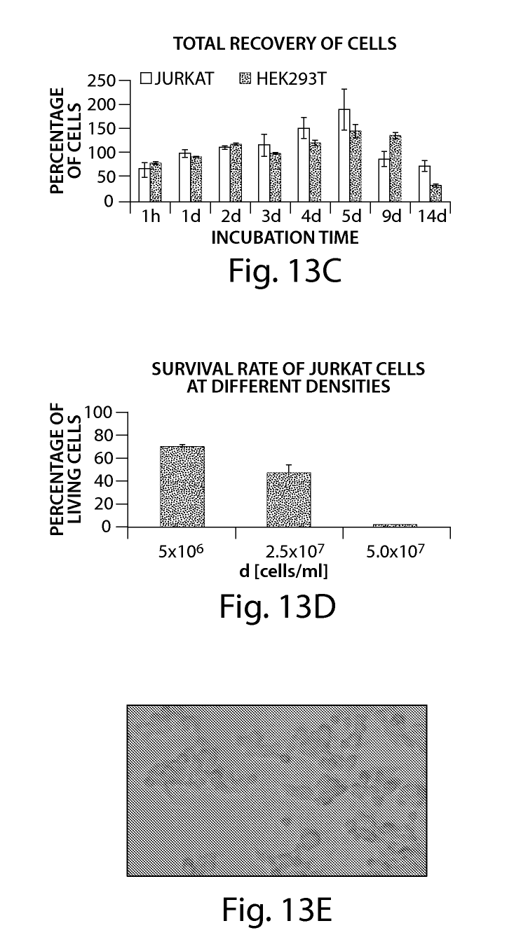

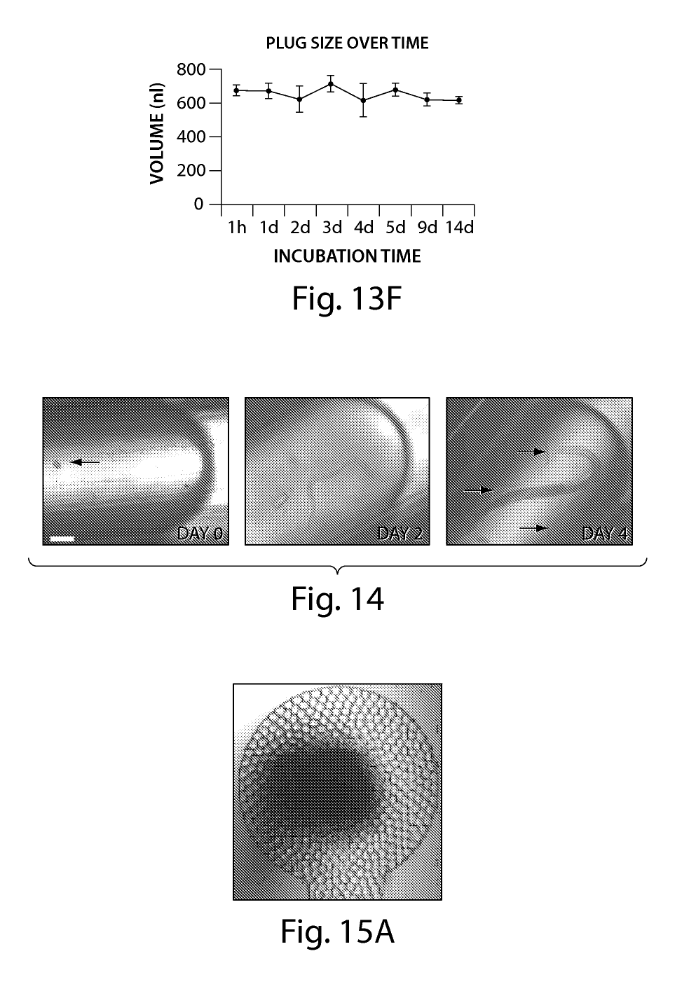

First, holding cartridges made of different materials were assessed for their suitability to host living cells. For this purpose 660 nl plugs each hosting 3300 Jurkat cells were generated. While gas-permeable PTFE tubing allowed cell survival for several days, the use of glass capillaries and vinyl tubing (all with an inner diameter of ˜0.5 mm) resulted in cell-death within 24 hours (data not shown). Live/dead stains revealed that when using PTFE tubing, the survival rate of Jurkat cells remained at approximately 90% for the first two days before decreasing gradually from 69% after three days, to 38% after five days and finally 6% after 14 days (FIG. 13A). The total number of recovered cells increased from 69% after 1 hour to 194% after 5 days indicating roughly 1-2 cell divisions (FIG. 13C). When repeating the experiments with adherent HEK293T cells, slightly different results were obtained (FIGS. 13B and 13C). Here, the fraction of viable cells remained above 80% for the first four days before slowly decreasing to 31% after 14 days. The recovery rate increased during the first five days from 83% to approximately 147%. Recultivation experiments demonstrated the recovery of fully viable and normally proliferating HEK293T cells after two days of encapsulation (FIG. 13E).

To assess the influence of the cell density on cell survival, experiments with 5- and 10-times more Jurkat cells per plug were also performed. Once again an inverse correlation between cell density and survival was obtained. While approximately 69% viable Jurkat cells were recovered after three days when using the initial cell density, only 52% and less than 1% survived when encapsulating five- and ten times more cells per plug, respectively (FIG. 13D). Not wishing to be bound by any theory, this massive decrease in cell survival may be due to the fact that higher cell densities directly resulted in more cells per plug (even at the lowest density all plugs were occupied), whereas when encapsulating single cells into drops the proportion of occupied drops was increased first (with a single cell in a drop still experiencing the same cell density).

In addition, an analysis was performed to determine whether the plugs were subjected to evaporation during the incubation period. For this purpose, the mean length of the plugs over time was determined by measuring the size of 30 plugs for each time point using a digital slide gauge and multiplying the mean value by the inner tube diameter to obtain the corresponding plug volumes. No significant decrease in size was observable (FIG. 13F), perhaps due to the fact that the incubation step was performed in a water-saturated atmosphere (at 37° C., 5% CO₂).

The possibility of encapsulating multicellular organisms was also investigated. Starting with eggs of the nematode C. elegans, plugs were analyzed under a microscope at different time points (FIG. 14). After two days, hatched worms had reached the L2-L3 larvae stage. Four days of encapsulation resulted in the growth of adult worms and the birth of the next generation (L1 larvae). Longer encapsulation resulted in plugs hosting up to 20 worms which finally died after 6-9 days. The passing of individual worms into adjacent microcompartments was never observed, even at high flow rates (up to 1000 microliters/h).

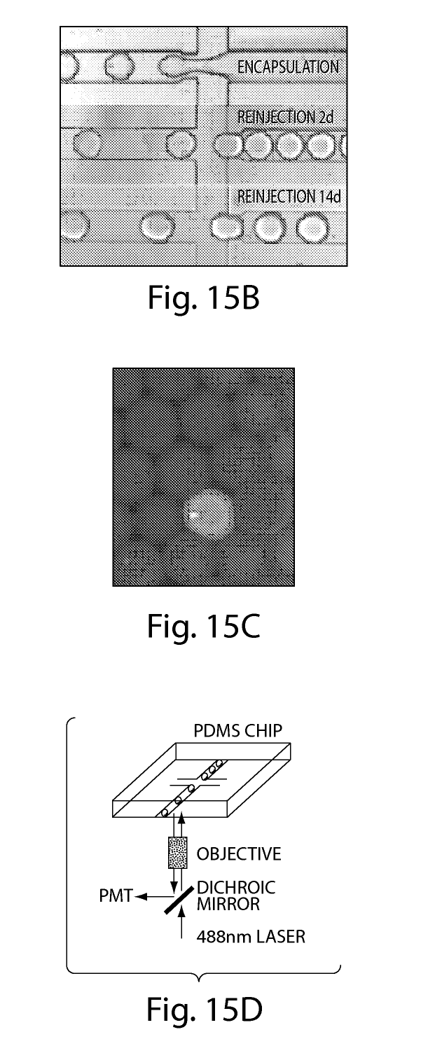

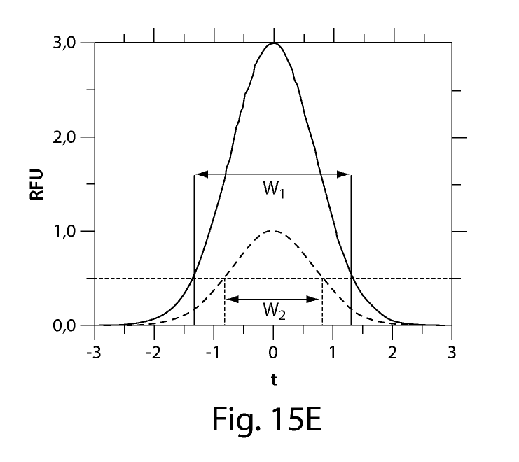

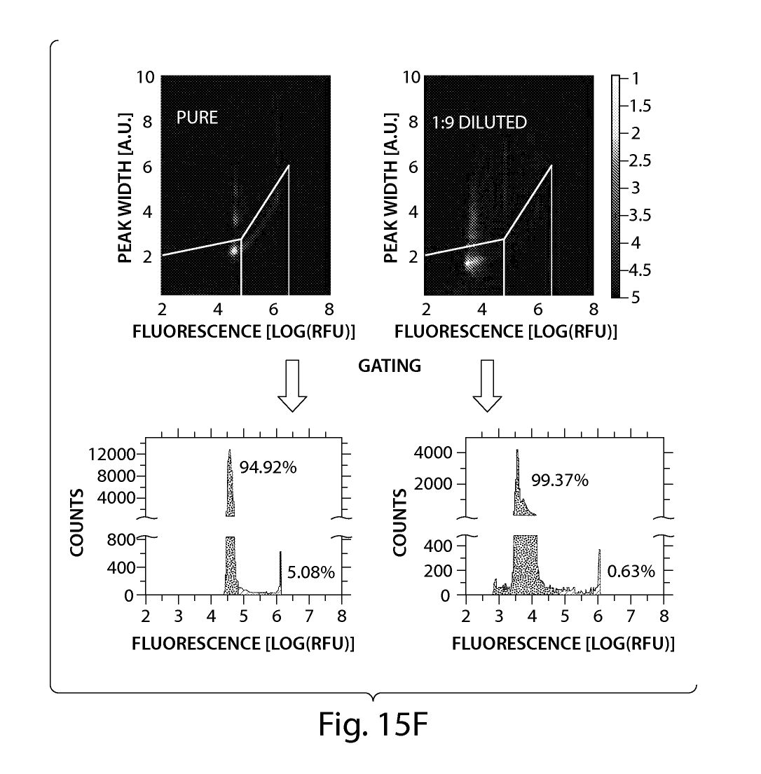

High-throughput cell-based assays require the readout of individual samples after the incubation step (e.g. to screen the phenotype of individual cells within a heterogeneous population). For this purpose, microcompartments stored in a piece of tubing or a reservoir were re-injected into an on-chip readout module after the incubation period. To prove the feasibility of this approach, HEK293T cells were encapsulated within 660 pl drops. The resulting emulsions were collected, and the samples were incubated for two and fourteen days. Subsequently, the emulsions were re-injected into a chip (same design as for the encapsulation step) and analyzed microscopically. During reinjection of the emulsion after two days of incubation, little coalescence of individual samples was detectable (FIG. 15A). After 14 days of incubation, some degree of coalescence was observable, however the majority of drops (>90%) remained intact. Microscopical comparison of the drops at the time of incubation and reinjection revealed no obvious reduction of the drop size (FIG. 15B). This indicates that the drops were not subjected to significant evaporation during the incubation period (within a water saturated atmosphere).

To demonstrate that the drops could be analyzed individually after reinjection, a population of HEK293T cells was encapsulated which, two weeks before the experiment, had been incubated in bulk with viral particles (murine leukemia virus pseudotyped with the G-protein of the vesicular stomatitis virus) having packaged the lacZ gene. The fraction of cells stably expressing the corresponding gene product (β-galactosidase) was approximately 13.9% as determined in an X-Gal assay. During the drop production a fluorogenic substrate (1.7 mM fluorescein di-β-D galactopyranoside, FDG) for β-galactosidase was co-encapsulated into the drops and a laser beam (488 nm wavelength) was focused onto the channel (downstream of the nozzle). The emitted light was collected in a photomultiplier (FIG. 15D) to record the fluorescence signal at t₀. This measurement was performed with the initial population of transduced HEK293T cells and a sample that had been diluted 1:9 with non-transduced HEK293T cells. At the time of encapsulation, no difference in the fluorescence signals was observable, and drops without any cells showed the same signal intensity (data not shown). After an incubation time of 16 hours at 37 degrees C., the emulsions were re-injected into the chip together with additional fluorinated oil (separately injected into the oil inlet to space out the drops) to repeat the fluorescence measurement (at tᵢ; analyzing 500 drops per second). Plotting the maximum fluorescence intensity of the drops against the peak width (which corresponds to the drop size and therefore is an indicator of coalescence) revealed different distinct populations (FIG. 15F). Analysis of the peak width proved that even though populations with two-fold and three-fold higher volumes were observable, the majority of drops did not coalesce (>93%). In terms of the fluorescence two main populations were obtained having a roughly 35-fold difference in their intensity, as also confirmed by fluorescence microscopy in which the drops appeared to be either highly fluorescent or non fluorescent (FIG. 15C). Based on these observations gates were set for the quantitative interpretation of the data (as routinely done in FACS analysis). Gates were set to analyze only the drops which had not coalesced (corresponding to the populations with the lowest peak width). Based on the way the peak width was defined fluorescence-positive drops appeared to be bigger (see FIG. 15E). Nonetheless, plotting the fluorescence against the peak width enabled non-coalesced drops to be clearly distinguished from coalesced drops for both species (positives and negatives). Using gating led to the conclusion that roughly 5.08% of all non-coalesced drops were fluorescence positive in the sample with non-diluted transduced cells. This number corresponded to approximately 12.7% of the corresponding cell population when taking into account that only 40.0% of the drops were occupied (as determined by microscopical analysis of the drops during the encapsulation step). This value was in the same range as the fraction of positive cells determined in bulk (˜13.9%), using a conventional X-Gal assay. For the diluted sample 0.63% positive drops were obtained, corresponding to 1.8% of the cells (34.8% of the drops were occupied). Compared to the non-diluted sample, the negative population showed a lower fluorescence intensity. Not wishing to be bound by any theory, this may have been due to the fact that all drops (even the ones without cells) contained traces of soluble β-galacosidase resulting from the few dead cells within the syringe (during the encapsulation step). Since the diluted sample contained less enzyme in total, a lower background could be expected, too. Another possible explanation would be the exchange of fluorescein between the drops. However, this explanation seems to be less likely, since for incubation periods of up to 24 hours, significant exchange of fluorescein were not observed for all surfactants tested (including the ammonium salt of carboxy-PFPE and PEG-PFPE; data not shown). The resulting 7.1-fold difference in terms of positive cells between the samples was in good agreement with the initial 1:9 dilution (assuming an accuracy of ±10% when counting the cultures in a Neubauer chamber before mixing leads to the conclusion that the effective ratio might have been as low as 1:7.4). In summary, these results clearly demonstrated the possibility of quantitatively analyzing individual drops in a high-throughput fashion (the drops were analyzed at a frequency of 500 Hz).

Droplet-based microfluidic systems have been used to create miniaturized reaction vessels in which both adherent and non-adherent cells can survive for several days. Even though microcompartments were generated with volumes of 660 pl and 660 nl only, in principal almost any volume could be generated by changing the channel sizes and flow rates, or by splitting relatively large microcompartments through a T-junction into smaller units. Thus microcompartments tailored for the encapsulation of small objects like single cells could be generated as well as compartments big enough to host multicellular organisms like C. elegans. Furthermore, the size could be adjusted according to the assay duration. Cell density was found to inversely correlate with the survival time of encapsulated cells. Larger compartments are hence preferential for long-term assays, especially since encapsulated cells proliferate within the microcompartments. Consequently even proliferation assays (e.g. for screening cytostatic drugs) should be possible as long as the chosen volume is big enough to guarantee sufficient supply of nutrition. On the other hand, small volumes might be advantageous for other applications, for example, to minimize reagent costs or to rapidly obtain high concentrations of secreted cellular factors. Besides the volume, further factors have been shown to have an impact on cell-survival, notably the biocompatibility of the surfactants and the gas-permeability of the storage system. Both non-ionic surfactants described herein allowed cell survival and proliferation, whereas the two ionic surfactants mediated cell-lysis. Even though there is no direct proof of correlation, it was striking that poly-L-lysine, a compound widely used to improve cell-attachment to surfaces, mediated membrane disruption when used as a head group of an ionic surfactant. Long-term incubation also requires sufficient gas-exchange. This can be ensured either by using open reservoirs, or channels or tubing made of gas-permeable materials such as fluorinated polymers. Efficient gas-exchange is also helped by the fact that perfluorocarbon carrier fluids can dissolve more than 20 times the amount of O₂, and three times the amount of CO₂, than water and have been shown to facilitate respiratory gas-delivery to both prokaryotic and eukaryotic cells in culture.

The possibility of re-injecting microcompartments into a chip after the incubation step opens the way for integrated droplet-based microfluidic systems for cell-based high-throughput screening. As has been shown here, a fluorescence-based readout of the expression of a cellular reporter gene can be performed in individual compartments at frequencies of 500 Hz. Hence a wide range of commercially-available fluorescence-based assays, can potentially be performed in a high throughput fashion. It is noteworthy that the possible coalescence of individual drops does not necessarily bias the readout. As shown here, coalesced drops with higher volumes can easily be identified and excluded from the data analysis. In theory, the use of gates also allows the analysis of solely those compartments hosting a specific number of (fluorescent) cells. In contrast to conventional FACS analysis the assay readout does not have to be based on fluorophores which remain in, or on the surface of the cells (e.g. GFP or fluorescent antibodies). Using compartmentalization, the activity of an intracellular reporter enzyme (β-galactosidase) has been measured using a fluorescent product that is highly membrane permeable (fluorescein).

The integration of additional microfluidic modules to the microfluidic platforms shown here allows the application range to be expanded. Integrating a microfluidic sorting module (based on dielectrophoresis or valves) could, for example, enable the screening of drug candidates. In the simplest case the candidates could be genetically-encoded by the encapsulated cells themselves (starting with a cell library): hence the collection of sorted positive drops would allow the identification of hits by DNA sequencing. Alternatively, the sorting module could be used to screen synthetic compounds fixed on beads (e.g. one-bead-one-compound libraries) co-encapsulated in the drops. After the sorting step, beads that mediated the desired effect could be recovered from the drops for a subsequent decoding step (e.g. by mass spectroscopy). Using optical barcodes encoding the compound identity might even allow the decoding step to be performed in real time (without the need for a sorting module). For example, different fluorescence channels could be used for the assay- and label-readout. The optical barcode does not have to be directly linked to the test compound when using droplet-based microfluidics: the label can simply be mixed with the test compound prior to the encapsulation step.

Aqueous microcompartments can be used as miniaturized vessels for chemical and biological reactions. It has been shown here how this approach can also be utilized for cell-based applications. It has been demonstrated that human cells, and even a multicellular organism (C. elegans), can be compartmentalized, and remain fully viable for several days in droplets. The microfluidic platforms described in this set of embodiments allow the encapsulation step at rates of more than 800 per second. As the number of cells per drop follows a Poission distribution the optional encapsulation of single cells causes the generation of empty drops thus decreasing the resulting encapsulation rate to about 300 per second. It has been demonstrated that post-incubation fluorescence readout of individual compartments at 500 Hz, and further droplet manipulation procedures (such as fusion, splitting and sorting) can be performed at similar rates. Consequently, the throughput of a single integrated droplet-based microfluidic system for cell-based screening could potentially be 500 times higher than conventional robotic microtitre-plate-based HTS technologies which can perform a maximum of ˜100,000 assays per day, or ˜1 s⁻¹. Using compartments as small as 660 pl, the volume of each assay, and hence the cost of reagents for screening, could be reduced by >1000-fold relative to the smallest assay volumes in microtitre plates (1-2 μl). This may allow many high-throughput biochemical screens to be replaced by more physiologically relevant cell-based assays, including assays using highly valuable cells, e.g. primary human cells, which are arguably the most physiologically relevant model systems, but which generally cannot be obtained on the scale required for HTS. The microfluidic device (FIG. 10A) was fabricated by patterning 75 μm deep channels into poly(dimethylsiloxane) (PDMS) using soft-lithography (Squires and Quake, 2005). The PDMS was activated by incubation for 3 minutes in an oxygen plasma (Plasma Prep 2, Gala Instrument) and bound to a 50 mm×75 mm glass slide (Fisher Bioblock). Inlets and outlets were made using 0.75 mm diameter biopsy punches (Harris Uni-Core). The channels were flushed with a commercial surface coating agent (Aquapel, PPG Industries) and subsequently with N2 prior to use.

HEK293T cells were grown and encapsulated in DMEM medium (Gibco), Jurkat cells were grown and encapsulated in RPMI medium (Gibco). Both media were supplemented with 10% fetal bovine serum (Gibco) and 1% penicillin/streptomycin (Gibco). Cells were incubated at 37° C. under a 5% CO2 atmosphere saturated with water.

For fluorescence readouts, the lacZ gene was introduced into HEK293T cells by retroviral transduction as described elsewhere (Stitz et al., 2001). In brief, by transfecting HEK293T cells murine leukemia virus-derived particles (pseudotyped with the G-protein of the vesicular stomatitis virus) were generated that had packaged a vector encoding lacZ. Two days after transfection the particles were harvested from the cell culture supernatants and used for transduction of fresh HEK293T cells during one hour of incubation. Subsequently the cells were cultivated for two weeks before encapsulating them together with 1.7 mM fluorescein di-β-D galactopyranoside (FDG, Euromedex) in drops.

In brief, surfactants (FIG. 11) were synthesized as follows:

Carboxy-PFPE. To obtain the ammonium salt of carboxy-PFPE, Krytox FS(L) 2000 (DuPont) was reacted with NH4OH as described (Johnston et al., 1996).

DMP-PFPE. Synthesis of the hydrophilic head group dimorpholinophosphate (DMP) was carried out by reaction of PhEtOH (Aldrich), POCl₃ (Fluka) and morpholine (Fluka) with (Et)3N (Sigma-Aldrich) in THF (Fluka) on ice. Subsequently DMP was coupled to water/cyclohexane/isopropanol extracted Krytox FS(H) 4000 (DuPont) by Friedels-Craft-Acylation.

PEG-PFPE. Reaction of Krytox FS(H) 4000 (DuPont) with polyethylene glycol (PEG) 900 (Sigma) resulted in a mixture of PEG molecules coupled to either one or two PFPE molecules.

poly L-Lysine-PFPE. Krytox FS(L) 2000 (DuPont) was reacted with poly L-Lysine (15,000-30,000; Sigma).

A 100 μl suspension of HEK293T cells (1.5×10⁶ cells/ml in fresh media) was seeded on top of a layer of perfluorocarbon oil (FC40, 3M) in the presence (0.5% w/w) and absence of the tested surfactants. After incubation at 37 degrees C. for 48 hours bright light images were taken using a Leica DMIRB microscope.

Cells were adjusted to a density of 2.5×10⁶ cells/ml (determined with a Neubauer counting chamber), stirred at 200 rpm using an 8 mm magnetic stir-bar (Roth) in a 5 ml polyethylene syringe (Fisher Bioblock), and injected via a PTFE tubing (0.56 mm×1.07 mm internal/external diameter, Fisher Bioblock) into the microfluidic device (FIG. 10A) using a syringe pump (PhD 2000, Harvard Apparatus) at a flow rate of 1000 microliters/h. The cell suspension was diluted on-chip (see below) by diluting with sterile media (1000 microliters/h if not otherwise stated) and drops were generated by flow-focusing of the resulting stream with perfluorinated oil (FC40, 3M), containing 0.5% (w/w) DMP-PFPE (4000 gl/h). The drop volume was calculated by dividing the flow rate by the drop frequency (determined using a Phantom V4.2 high speed camera). Experimental variations in the drop frequency (at constant flow rates) were defined as the degree of polydispersity in terms of the volume (corresponding to the third power of the polydispersity in terms of the diameter when considering a perfect sphere). For each sample, 500 microliters of the resulting emulsion were collected within a 15 ml centrifuge tube and incubated at 37 degrees C. within a CO₂ incubator (5% CO₂, saturated with H₂O). After incubation, 250 microliters of the emulsion was transferred into a new centrifuge tube and broken by the addition of 15% Emulsion Destabilizer A104 (RainDance Technologies, Guilford, Conn.) and 10 ml of live/dead staining solution (LIVE/DEAD Viability/Cytotoxicity Kit for animal cells, Invitrogen Kit L-3224) and subsequent mixing. After incubation for three minutes (to allow sedimentation of the oil phase) the supernatant was transferred into a 25 cm² tissue culture flask and incubated one hour at room temperature.

Drops were generated and diluted on-chip by bringing together two channels containing the cell suspension and sterile media respectively and varying the relative flow rates while keeping the overall aqueous flow rate constant at 2000 microliters/h using two syringe pumps. The number of cells per drop was determined by evaluating movies taken with a high speed camera (Phantom V4.2) mounted on a microscope. For each dilution, 120 drops were analyzed to determine the number of cells per drop. Subsequently the data was fitted to a Poisson distribution (p(x=k)=e-λ×λk/k!) using XmGrace (http://plasma-gate.weitzmann.ac.il/grace).

The emulsions were collected in open syringes (without the plunger being inserted) and incubated within a water-saturated atmosphere (37 degrees C., 5% CO₂). During the encapsulation step, a laser beam (488 nm wavelength) was focused onto the channel using an objective with a 40-fold magnification (FIG. 15D, downstream of the nozzle) to excite the fluorophore. Emitted light was diverted by a dichroic mirror (488 nm notch filter), filtered (510 nm±10 nm) and collected in a photomultiplier to record the first fluorescence measurement (t₀). After the desired incubation time mineral oil was added to fill the syringe completely before inserting the plunger and re-injecting the emulsion together with 0.5% w/w DMP-PFPE surfactant in FC40 (injected into the oil inlet to space out the drops) into a chip with the same design as for the encapsulation step. To avoid fragmentation of the drops before the second fluorescence measurement (at tᵢ) the flow direction was reversed compared to the encapsulation step (the emulsion was injected into the outlet (FIG. 10A) to avoid branching channels). All signals from the photomultiplier were recorded using Labview (National Instruments) running an in-house program for the data analysis.

To prepare the plugs 5×10⁶ cells/ml (determined with a Neubauer counting chamber) were stirred at 510 rpm within a 1.8 ml cryotube (Nunc) using an 8 mm magnetic stir-bar (Roth) and kept at 4 degrees C. Subsequently 660 nl plugs of this cell suspension and perfluorinated oil (FC40, 3M) were aspirated (at 500 microliters/h) into PTFE tubing (0.56 μm×1.07 mm internal/external diameter, Fisher Bioblock) in an alternating fashion using a syringe pump (PhD 2000, Harvard Apparatus). For each sample, 30 plugs were loaded before the tubing was sealed (by clamping microtubes to both ends) and incubated at 37 degrees C. within a CO₂ incubator (5% CO₂, saturated with H₂O). After incubation, the plugs were infused into a 25 cm² tissue culture flask. Subsequently 4 ml of live/dead staining solution (LIVE/DEAD Viability/Cytotoxicity Kit for animal cells, Invitrogen Kit L-3224) were added and the samples were incubated for one hour at room temperature. When using adherent cells, the staining solution was additionally supplemented with 0.25 g/l trypsin (Gibco) to break up cell clumps.

After staining, live and dead cells were counted manually using a microscope (Leica DMIRB) with a UV-lightsource (LEJ ebq 100). For each sample within a 25 cm² tissue culture flask 30 fields of view (corresponding to ˜4.2 mm²) were evaluated to calculate the total number of living (green stain) and dead (red stain) cells.

Eggs were resuspended in M9 minimal media (Sigma) supplemented with E. coli OP50 (10% w/v of pelleted bacteria). Plugs of the resulting suspension were aspirated into PTFE tubing and incubated at room temperature.

For recultivation of cells recovered from drops or plugs, semi-conditioned media supplemented with 30% fetal bovine serum (Gibco) was added to the cells instead of the staining solution. Cells were then incubated for two days at 37 degrees C. within a CO₂ incubator (5% CO₂, saturated with H₂O) before imaging using bright-field microscopy.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of and “consisting essentially of” shall be closed or semi-closed transitional phrases, respectively, as set forth in the United States Patent Office Manual of Patent Examining Procedures, Section 2111.03.

What is claimed is:

1-225. (canceled)

226. A method of determining binding of a protein to a particle contained within a fluidic droplet, the method comprising acts of:
(a) providing a microfluidic channel containing a fluid containing microfluidic droplets having an average diameter of less than about 1 mm, wherein at least one microfluidic droplet contains: (1) a protein-producing cell, and (2) a particle having immobilized thereon a protein-binding agent;
(b) causing the protein-producing cell within the microfluidic droplet to secrete, into the fluid, a protein;
(c) causing binding of the secreted protein in the fluid to the particle via the protein-binding agent; and
(d) determining at least one characteristic of the secreted protein by determining a characteristic of the particle contained in the droplet, after binding of the protein to the protein-binding agent immobilized on the particle.

227. The method of claim 226, further comprising sorting the microfluidic droplet based on the determined at least one characteristic of the protein.

228. The method of claim 226, wherein the protein-producing cell is a blood cell.

229. The method of claim 228, wherein the blood cell is removed from a subject.

230. The method of claim 226, wherein the protein-producing cells are encapsulated in the plurality of microfluidic droplets at an average ratio of no more than about 1 cell/microfluidic droplet.

231. The method of claim 226, wherein the protein is an antibody.

232. The method of claim 231, wherein the droplet further comprises a target to which the antibody is able to bind, and a signaling entity able to bind the target.

233. The method of claim 232, further comprising determining binding of the antibody to the binding agent and the target, thereby determining selectivity of binding of the antibody to the target.

234. The method of claim 226, wherein the microfluidic droplets are substantially monodisperse.

235. The method of claim 226, further comprising cloning DNA from the protein-producing cell.

236. The method of claim 235, wherein the DNA is amplified prior to cloning.

237. The method of claim 235, further comprising inserting at least a portion of the DNA in a host cell, and culturing the host cell to express the DNA.

238. The method of claim 226, further comprising:
sequencing DNA from the protein-producing cell; and
inserting at least a portion of the DNA in a host cell.

239. The method of claim 226, wherein the particle is fluorescent.

240. The method of claim 226, wherein the protein-binding agent is an antibody.

241. The method of claim 226, wherein the protein-binding agent is able to specifically bind the protein.

242. The method of claim 226, wherein the protein-producing cell is non-immortal.

243. The method of claim 226, wherein the protein-producing cell is a human cell.

244. A method, comprising:
removing blood cells from a subject;
encapsulating at least some of the blood cells in a plurality of aqueous microfluidic droplets contained within a fluid immiscible with the droplets, contained within a microfluidic channel, wherein the microfluidic droplets are substantially monodisperse and have an average diameter of less than about 1 mm;
at least partially separating, from the plurality of fluidic droplets, droplets containing antibody-producing cells from droplets that do not contain antibody-producing cells;
culturing the antibody-producing cells within the separated microfluidic droplets to secrete antibodies;
exposing the antibodies within the separated microfluidic droplets to a signaling entity comprising a microparticle and an agent, immobilized relative to the microparticle, able to bind the antibodies;
determining a characteristic of the antibodies by determining a change in a characteristic of the signaling entity in the presence of the antibodies contained within the microfluidic droplets; and
sorting the microfluidic droplets based on the determined characteristic of the antibodies produced by the antibody-producing blood cells.

245. A method, comprising:
encapsulating antibody-producing cells in a plurality of aqueous microfluidic droplets contained within a microfluidic channel, wherein the microfluidic droplets are substantially monodisperse and have an average diameter of less than about 1 mm;
culturing the antibody-producing cells within the microfluidic droplets to secrete antibodies;
exposing the secreted antibodies to a signaling entity contained within the microfluidic droplets, wherein the signaling entity comprises a microparticle and an agent, immobilized relative to the microparticle, able to bind the antibodies;
determining a characteristic of the antibodies by determining a change in a characteristic of the signaling entity in the presence of the antibodies contained within the microfluidic droplets; and
sorting the microfluidic droplets based on the determined characteristic of the antibodies produced by the antibody-producing blood cells.


SEQUENCE LISTING (1 sequence)

SEQ ID NO: 1 - protein, 6 aa, Artificial Sequence
  Synthetic Peptide
CCPGCC